SEQUENCE LISTING (41 sequences)

SEQ ID NO: 1 - protein, 556 aa, Homo sapiens [sequence omitted]

SEQ ID NO: 2 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 4 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 5 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 6 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 7 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 8 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 9 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 10 - protein, 448 aa, Homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 11 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 12 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 13 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 14 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 15 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 16 - protein, 1210 aa, Homo sapiens [sequence omitted]

SEQ ID NO: 17 - protein, 1255 aa, Homo sapiens [sequence omitted]

SEQ ID NO: 18 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 19 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 20 - protein, 263 aa, Homo sapiens
MFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 21 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 22 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 23 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 24 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 25 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 26 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 27 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 28 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 29 - protein, 9 aa, Homo sapiens
SLLMWITQC

SEQ ID NO: 30 - protein, 9 aa, Homo sapiens
SLLMWITQV

SEQ ID NO: 31 - protein, 9 aa, Homo sapiens
RMFPNAPYL

SEQ ID NO: 32 - protein, 9 aa, Homo sapiens
KVAELVHFL

SEQ ID NO: 33 - protein, 9 aa, Homo sapiens
EVDPIGHLY

SEQ ID NO: 34 - protein, 9 aa, Homo sapiens
NYKRCFPVI

SEQ ID NO: 35 - protein, 9 aa, Homo sapiens
AAGIGILTV

SEQ ID NO: 36 - protein, 9 aa, Homo sapiens
YMLDLQPET

SEQ ID NO: 37 - protein, 15 aa, Homo sapiens
LKEFTVSGNILTIRL

SEQ ID NO: 38 - protein, 20 aa, Homo sapiens
LPVPGVLLKEFTVSGNILTI

SEQ ID NO: 39 - protein, 15 aa, Homo sapiens
TSYVKVLHHMVKISG

SEQ ID NO: 40 - protein, 23 aa, Homo sapiens
RNGYRALMDKSLHVGTQCALTRR

SEQ ID NO: 41 - protein, 10 aa, Homo sapiens
ALYVDSLFFL

(19) United States
(12) Patent Application Publication
(10) Pub. No.: US 2022/0249562 A1
(43) Pub. Date: Aug. 11, 2022

(54) TYPE III NKT CELLS AND RELATED COMPOSITIONS AND METHODS

(71) Applicant: Akeso Therapeutics, Inc., Yorktown Heights, NY (US)

(72) Inventors: Xianzheng Zhou, Yorktown Heights, NY (US); Xin Huang, Yorktown Heights, NY (US)

(21) Appl. No.: 17/618,006

(22) PCT Filed: Aug. 20, 2020
(86) PCT No.: PCT/US20/47253

§ 371 Date: Dec. 10, 2021

Related U.S. Application Data
(60) Provisional application No. 62/889,664, filed on Aug. 21, 2019.

(51) Int. Cl. A61K 35/17; C12N 5/0783; A61P 35/00

(52) U.S. Cl. CPC A61K 35/17; A61P 35/00; C12N 5/0646

(57) ABSTRACT

The present disclosure relates to type III natural killer T (NKT) cells (e.g., CD3⁺CD56⁺ type III NKT cells), pharmaceutical compositions, and methods of preparation and use thereof, in particular use of them as therapeutic agents for the treatment various cancers. Modified type III NKT cells, e.g., to express a chimeric antigen receptor (CAR), a T cell receptor (TCR), a T cell receptor mimic antibody (TCRm), or a combination thereof, pharmaceutical compositions, and methods of preparation and use thereof, are also disclosed.

### CROSS-REFERENCE TO RELATED APPLICATIONS

This application claims priority under 35 U.S.C. § 119(e) to U.S. Provisional Patent Application No. 62/889,664, filed on Aug. 21, 2019, the disclosure of which is incorporated herein by reference in its entirety.

### FIELD OF THE DISCLOSURE

The present disclosure relates to type III natural killer T (NKT) cells (e.g., CD3⁺CD56⁺ type III NKT cells), in particular modified cells, pharmaceutical compositions of the type III NKT cells, methods of preparation, and therapeutic use of the cells or pharmaceutical compositions thereof for treatment or prevention of diseases and disorders, including various cancers.

### BACKGROUND OF THE DISCLOSURE

Natural killer T (NKT) cells are a T-cell subset that exhibits characteristics of both conventional T cells and natural killer (NK) cells. NKT cells typically arise in the thymus from CD4⁺CD8⁺ cortical thymocytes that have undergone T cell receptor (TCR) gene rearrangement. NKT cells have been traditionally defined as CD1d-restricted, lipid antigen-reactive T cells and classified as type I and type II NKT cells (Godfrey et al., Immunity 2018; 48(3):453-73; Dhodapkar and Kumar, J Immunol. 2017; 198(3):1015-21. Based on their TCR repertoire, antigen specificity and CD1d dependence, NKT cells have also been divided into three categories: type I, type II and type III NKT cells (Godfrey et al., Nat Rev Immunol. 2004; 4(3):231-37).

Type I or invariant NKT (iNKT) cells express an invariant TCRα-chain (TRAV11 and TRAJ18 in mice and TRAV10 and TRAJ18 in humans) and a limited number of non-invariant TCRβ-chains. Type I NKT cells also recognize the glycosphingolipid α-galactosylceramide (α-GalCer) antigen when presented by major histocompatibility complex (MHC) class I-like CD1d molecules. Type II NKT cells have a more diverse and less well-defined TCR repertoire and recognize non-α-GalCer molecules (such as sulfatide) presented by CD1d molecules. Type III NKT or NKT-like cells have a diverse TCR repertoire and recognize CD1d-independent molecules.

Type I NKT cells have been intensively studied and are known for the paradoxical ability to either promote or suppress cell-mediated immunity due to diverse cytokine-secreting subsets. No specific markers for the type II NKT cell population have been identified, and the functional role of type II NKT cells remains unclear and are largely considered to be immunosuppressive in murine studies (Dhodapkar and Kumar, J Immunol. 2017; 198(3):1015-21; Marrero et al., Front Immunol. 2015; 6:316; Kato et al., Front Immunol. 2018; 9:314). Type III NKT cells are by far the most heterogeneous and the least characterized in mice and humans (Farr et al. Proc Natl Acad Sci USA. 2014; 111(35):12841-46; Yu et al. J Clin Invest. 2011; 121(4):1456-70).

Like iNKT cells, type III NKT cells also develop in the thymus independent of MHC class I or class II molecules. Molecular and functional evidence suggests that CD1d-unrestricted type III NKT cells in mice are uniquely programmed with a hybrid of function of both innate (like NK cells) and adaptive immunity (like T cells) far superior than that of iNKT cells. Global genome expression reveals higher similarities between type III NKT cells and NK cells than between type III NKT cells and iNKT cells (Farr et al. Proc Natl Acad Sci USA. 2014; 111(35):12841-46).

Type III NKT cells have been postulated to play an important role in anti-tumor and anti-virus immune response (Lu et al. J Immunol. 1994; 153(4):1687-96; Kokordelis et al. J Acquir Immune Defic Syndr. 2015; 70(4):338-46). In human cancers, including lung, colorectal cancer and gastric cancer, high levels of type III NKT are associated with improved patient's survival (Pan et al. Tumor Biol. 2014:35(1):701-7; Bojarska-Junak et al. Oncol Rep. 2010:24(3):803-10; Peng et al. Oncotarget. 2016:7(34):55222-30).

Double negative T (DNT) cells are CD3⁺CD56⁻, CD4⁻CD8⁻ mature T cells. It has been shown that ex vivo expanded DNT from AML patients or healthy donors induce potent cytotoxicity and IFN-γ production against primary AML cells and chemotherapy-resistant leukemia and reduce leukemia load in patient-derived xenograft (PDX) models (Lee et al. Clin Cancer Res. 2018; 24(2):370-82; Lee et al. Clin Cancer Res. 2019; 25(7):2241-53). Furthermore, allogeneic DNT from healthy donors do not cause xenogeneic graft versus host disease (GvHD), supporting the use of DNT cells as off-the-shelf cellular therapy. However, the cellular components in DNT cells that mediate potent anti-acute myeloid leukemia (AML) activity are not known.

Acute myeloid leukemia (AML), also known as acute myelogenous leukemia, is a cancer of the myeloid line of blood cells, characterized by the rapid growth of abnormal white blood cells that accumulate in the bone marrow and interfere with the production of normal blood cells. As an acute leukemia, AML progresses rapidly and is typically fatal within weeks or months if left untreated. AML is the most prevalent form of adult leukemia, particularly among the elderly and is slightly more common in men than women. In 2018, an estimated 19,520 new cases and 10,670 deaths occurred in the US (Siegel et al., CA Cancer J Clin. 2018; 68:7-30). The disease is particularly difficult to treat in older adults who account for the majority of patients; thus, the 5-year overall survival is only approximately 27% (National Cancer Institute. SEER Cancer stat facts: acute myeloid leukemia (AML)).

In recent decades, data detailing the molecular ontogeny of AML have elucidated causal pathways and led to improved chemotherapies and targeted drug development (Lindsley et al., Blood 2015; 125:1367-1376; Papaemmanuil et al., N Engl J Med. 2016; 374:2209-2221). Such therapies, however, can yield moderate overall response and/or low complete response rates. Thus, there remains a need for effective cancer therapies, particularly those targeting hard-to-treat cancers such as AML.

### SUMMARY OF THE DISCLOSURE

The present disclosure relates to type III natural killer T (NKT) cells (e.g., CD3⁺CD56⁺ type III NKT cells). In some embodiments, the type III NKT cells disclosed herein are CD3⁺CD56⁺ type III NKT cells (e.g., CD3⁺CD4⁺CD56⁺ cells, CD3⁺CD8⁺CD56⁺ cells, CD3⁺CD4⁻CD8⁻CD56⁺ cells or a mixture thereof). In some embodiments, the type III NKT cells disclosed herein are modified, e.g., to express a chimeric antigen receptor (CAR), a T cell receptor (TCR), a T cell receptor mimic antibody (TCRm), or a combination thereof.

The present disclosure further relates to methods and compositions (e.g., pharmaceutical compositions) using the type III NKT cells disclosed herein. In some embodiments, the type III NKT cells disclosed herein may be useful as therapeutic agents, e.g., in treating or preventing a cancer (e.g., acute myeloid leukemia (AML)). In some embodiments, the type III NKT cells disclosed herein may be isolated (e.g., from a biological sample, e.g., from a patient or a donor), cultured, and/or expanded into a cell population. In some embodiments, the type III NKT cells disclosed herein are present and/or used in a pharmaceutical composition.

Pharmaceutical compositions comprising the type III NKT cells disclosed herein are also provided. In one aspect, the disclosure relates to a pharmaceutical composition comprising isolated CD3⁺CD56⁺ type III natural killer T cells according to any embodiment discloses here and a pharmaceutically acceptable carrier. In some embodiments, the cells are CD3⁺CD4⁺CD56⁺, CD3⁺CD8⁺CD56⁺, or CD3⁺CD4⁻CD8⁻CD56⁺ cells, each optionally isolated from a biological sample of the subject or a donor.

In one embodiment, the disclosure relates to a method of treating or preventing a cancer in a subject in need thereof, comprising administering to the subject a therapeutically effective amount of CD3⁺CD56⁺ type III natural killer T cells according to any embodiment disclosed here, or a pharmaceutical composition comprising a therapeutically effective amount of CD3⁺CD56⁺ type III natural killer T cells and a pharmaceutically acceptable carrier.

In another embodiment, the disclosure relates to a method of preparing a therapy for treating or preventing a cancer in a subject in need thereof, comprising:

a) isolating one or more CD3⁺CD56⁺ type III natural killer T cells from a biological sample; and

b) culturing the one or more CD3⁺CD56⁺ type III natural killer T cells in a growth medium to produce an expanded cell population.

In some embodiments, the method further includes modifying the one or more CD3⁺CD56⁺ type III natural killer T cells to express a CAR, TCR, or TCRm. In some embodiments, the modifying comprises introducing one or more polynucleotides encoding the CAR, TCR, or TCRm into the one or more cells.

In any of the aspects, the cancer that the isolated cells can be used to treat or prevent includes, but not limited to, solid tumor or a hematological malignancy; B-cell malignancy, leukemia, lymphoma, myeloma, or melanoma; acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma.

Other aspects or advantages of the present disclosure can be better understood through the following description of drawings, detailed description of disclosure, examples, and claims.

### BRIEF DESCRIPTION OF THE DRAWINGS

FIG. 1A is a flow cytometric analysis plot showing phenotypes of double negative T cells (DNT) pre- and post-culture. DNT cells were enriched from PBMCs of a healthy donor using CD4 and CD8 depletion cocktails and cultured in anti-CD3 antibody-coated plates (OKT3; 5 μg/mL) and IL-2 (250 IU/mL) for 3 days. Soluble anti-CD3 (0.1 μg/mL) was added on days 7, 10, and 14. Fresh media and IL-2 were added on days 3, 7, and 10. As a control, DNT cells were activated with Dynabeads Human T-Activator CD3/CD28 for 3 days and subsequently cultured with IL-2 (50 IU/mL).

FIG. 1B is a flow cytometric analysis plot showing anti-AML cytotoxicity and IFN-γ production in DNT cultures gated on CD3⁺CD4⁻CD8⁻ populations. Expression of cell surface CD107a indicates cell cytotoxicity. CD19 CAR-T (37% CAR⁺) and PMA/ionomycin were used as a control. Other target cells included K562 (erythroleukemia), K562CD19 (CD19 transfected K562), Nalm-6 (B-ALL, B cell precursor leukemia), Raji (Burkitt's lymphoma), and U937 (AML).

FIG. 2A is a graph showing an exemplary optimization of a luciferase-based killing assay using CD19 CAR-T cells at various effector:target (E/T) ratios.

FIG. 2B is a graph showing an exemplary optimization of a luciferase-based killing assay using CD19 CAR-T cells. CD19⁺ target cells included Daudi (Burkitt's lymphoma), Nalm-6 (B-ALL) and Raji (Burkitt's lymphoma).

FIG. 2C is a graph showing release of IFN-γ by CD19 CAR-T cells following recognition of CD19⁺ target cells.

FIG. 2D is a graph showing an exemplary validation of a luciferase-based killing assay using IGF1R CAR-T and ROR1 CAR-T against sarcoma cell lines.

FIG. 2E is a graph showing an exemplary validation of a luciferase-based killing assay using IGF1R CAR-T and ROR1 CAR-T against sarcoma cell lines.

FIG. 2F is a graph showing release of IFN-γ by IGF1R CAR-T and ROR1 CAR-T cells following recognition of antigen-expressing sarcoma cell lines.

FIG. 3A is a graph showing phenotypes of T cell clones 2A (DNT), 3F (CD4⁺ NKT) and 4E (CD8⁺ NKT). Phenotypes indicate CD3⁺CD4⁻CD8⁻CD56⁻ DNT, CD3⁺CD4⁺CD56⁺ NKT, and CD3⁺CD8⁺CD56⁺ NKT cells, respectively.

FIG. 3B is a graph showing the frequency of T cell receptor (TCR) Vβ repertoire of 2A, 3F, and 4E clones using a TCR Vβ3 Repertoire Kit and an iNKT TCR Vα24-Jα1 (iTCR) antibody. Gray shade dot line, isotype antibody; solid line, anti-iTCR antibody.

FIG. 3C is a graph showing lysis of U937 AML target cells by 2A, 3F, and 4E clones in a 4-h luciferase-based killing assay. Target cells were transduced with lentivirus encoding humanized luciferase and truncated nerve growth factor receptor (ΔNGFR) (hfflucN) and enriched for NGFR⁺ cells by biotin labelled anti-NGFR antibody and anti-biotin microbeads.

FIG. 3D is a graph showing production of IFN-γ by 2A, 3F, and 4E clones following recognition of a panel of AML cell lines (including KG-1, Molm-13, Molm-14, MV4:11, U937) in an enzyme-linked immunosorbent assay (ELISA). Supernatants from T cell and target cell co-cultures in duplicate were collected at 24 h to measure IFN-γ levels.

FIG. 3E is a flow cytometric analysis plot showing the cytotoxicity of 2A, 3F, and 4E clones on tested AML cell lines by levels of CD107a expression and intracellular IFN-γ.

FIG. 4A is a graph showing the cytotoxicity of 2A, 3F, and 4E clones on luciferase-expressing AML cells. HL-60mx, HL-60 selected to Mitoxantrone resistance.

FIG. 4B is a graph showing release of IFN-γ by 2A, 3F, and 4E clones in an ELISA IFN-γ assay in response to luciferase-expressing AML cells.

FIG. 4C is a graph showing cytotoxicity of 2A, 3F, and 4E clones on sarcoma cell lines (SaOs2, TC71, Rh30).

FIG. 4D is a graph showing statistical analysis of five independent cytotoxicity assays of 2A, 3F, and 4E clones on luciferase-expressing AML cell lines (HL60, KG-1, Molm-13, Molm-14, MV4:11, THP-1, U937). The results were calculated as means±SD. *, p <0.05, **, p<0.01, ***, p<0.001, ****, p<0.0001, ns, not significant.

FIG. 4E is a graph showing statistical analysis of two independent ELISA assays of release of IFN-γ by 2A, 3F, and 4E clones in response to luciferase-expressing AML cells (HL60, KG-1, Molm-13, Molm-14, MV4:11, THP-1, U937). The results were calculated as means±SD. *, p<0.05, **, p<0.01, ***, p<0.001, ****, p<0.0001.

FIG. 5A is a flow cytometric analysis plot showing the percentages of CD3⁺CD56⁺ type III NKT cells in PBMCs from two healthy blood donors after purification using a CD3⁺CD56⁺ NKT Cell Isolation Kit.

FIG. 5B is a graph showing the percentages of CD3⁺CD56⁺ type III NKT cells and CD4⁺, CD8⁺, and CD4⁻CD8⁻ cells after gating of CD3⁺CD56⁺ and CD3⁺CD56⁻ populations following activation of purified CD3⁺CD56⁺ NKT cells with Dynabeads Human T-Activator CD3/CD28.

FIG. 5C is a graph showing the percentages of iNKT TCR Vα24-Jα1 positive cells in cultured CD3⁺CD56⁺ NKT cells.

FIG. 5D is a graph showing luciferase-based lysis assays conducted on 2A, 3F, and 4E clones, and CD3⁺CD56⁺ NKT cells from healthy blood donor 1 (NKT1), at various effector:target (E/T) ratios.

FIG. 5E is a graph showing production of IFN-γ by 2A, 3F, and 4E clones, and NKT1 cells, following recognition of U937 AML, Nalm-6 (B-ALL), or Jurkat (T-ALL) cells.

FIG. 6A is a flow cytometric analysis plot showing the percentages of CD3⁺CD56⁺ NKT cells in PBMCs from two healthy blood donors (PBMC3 and PBMC4) after purification using a CD3⁺CD56⁺ NKT Cell Isolation Kit.

FIG. 6B is a graph showing the percentages of CD3⁺CD56⁺ NKT cells following activation of purified CD3⁺CD56⁺ NKT cells with Dynabeads Human T-Activator CD3/CD28 (NKT3 beads) on day 3 and expanded with OKT3 (muromonab-CD3, Orthoclone OKT3) on day 15.

FIG. 6C is a graph showing cytotoxicity of CD3⁺CD56⁺ NKT cells (which were isolated from healthy blood donor 2 (NKT2), activated with Dynabeads Human T-Activator CD3/CD28, and expanded with OKT3) on a panel of AML cells in a luciferase-based killing assay.

FIG. 6D is a graph showing cytotoxicity of CD3⁺CD56⁺ NKT cells (which were isolated from healthy blood donor 9, 10, 11 and 12, activated with Dynabeads Human T-Activator CD3/CD28, and expanded with OKT3) on a panel of AML cells in a luciferase-based killing assay. OKT3 was purchased from BioCell.

FIG. 6E is a graph showing statistical analysis of cytotoxicity of CD3⁺CD56⁺ NKT cells on luciferase-expressing AML (HL60, KG-1, Molm-13, Molm-14, THP-1, U937) and B-cell malignancies (Daudi, Raji, Nalm-6) at E/T ratios of 30:1, 10:1 and 3.3:1. CD3⁺CD56⁺ NKT cells were isolated from six healthy blood donors (NKT1, NKT2, NKT9, NKT10, NKT11, NKT12), activated with Dynabeads Human T-Activator CD3/CD28, and expanded with OKT3. The results were calculated as means±SD. *, p<0.05, **, p<0.01, ***, p<0.001, ****, p<0.0001.

FIG. 6F is a graph showing statistical analysis of cytotoxicity of CD3⁺CD56⁺ NKT cells on luciferase-expressing AML (HL60, KG-1, Molm-13, Molm-14, THP-1, U937) and B-cell malignancies (Daudi, Raji, Nalm6) at E/T ratios of 15:1. 5:1 and 1.67:1. CD3⁺CD56⁺ NKT cells were isolated from four healthy blood donors (NKT9, NKT10, NKT11, NKT12), activated with Dynabeads Human T-Activator CD3/CD28, and expanded with OKT3. The results were calculated as means±SD. *, p<0.05, **, p<0.01, ***, p<0.001, ****, p<0.0001, ns, not significant.

FIG. 7A is a graph showing cytotoxicity of CD3⁺CD56⁺ NKT cells (which were isolated from healthy blood donor 2, thawed from a frozen vial, activated in a OKT3-coated T25 flask with IL-2, and expanded with OKT3 plus IL-2) on a panel of AML cells in a luciferase-based killing assay. NKT2 on day 17 and 22 after OKT3 activation were used in this assay.

FIG. 7B is a graph showing cytotoxicity of CD3⁺CD56⁺ NKT cells (which were isolated from healthy blood donor 2 and 17, activated in OKT3-coated T25 flasks with IL-2, and expanded with OKT3 plus IL-2) on a panel of AML cells in a luciferase-based killing assay. NKT2 on day 41 and NKT17 on day 18 after OKT3 activation were used in this assay.

FIG. 7C is a graph showing cytotoxicity of CD3⁺CD56⁺ NKT cells (which were isolated from healthy blood donor 20, 21 and 22, activated in OKT3-coated T25 flasks with IL-2, and expanded with OKT3 plus IL-2) on a panel of AML cells in a luciferase-based killing assay.

FIG. 7D is a graph showing cytotoxicity of CD3⁺CD56⁺ NKT cells (which were isolated from healthy blood donor 27, 30 and 31, activated in OKT3-coated 24-well plates with IL-2, and expanded with OKT3 plus IL-2) on a panel of AML cells in a luciferase-based killing assay. OKT3 was purchased from Miltenyi Biotec.

FIG. 7E is a graph showing cytotoxicity of CD3⁺CD56⁺ NKT cells (which were isolated from healthy blood donor 30, 31 and 32, activated in OKT3-coated 24-well plates with IL-2, and expanded with OKT3 plus IL-2) on a panel of AML cells in a luciferase-based killing assay.

FIG. 7F is a graph showing cytotoxicity of CD3⁺CD56⁺ NKT cells (which were isolated from healthy blood donor 35, activated in OKT3-coated 24-well plates with IL-2, and expanded with OKT3 plus IL-2) on a panel of AML cells in a luciferase-based killing assay.

FIG. 7G is a Table 1 showing statistical analysis of cytotoxicity of CD3⁺CD56⁺ NKT cells isolated from nine healthy donors (n=9).

FIG. 7H is a graph showing statistical analysis of cytotoxicity of CD3⁺CD56⁺ NKT cells on luciferase-expressing AML (HL60, KG-1, Molm-13, THP-1, U937) and B-cell malignancies (Daudi, Raji, Nalm6) at merged E/T ratios of 20:1, 6.7:1, 2.2:1 (n=4), and 15:1, 5:1, 1.7:1 (n=5). Those CD3⁺CD56⁺ NKT cells were activated in OKT3-coated 24-well plates with IL-2, and expanded with OKT3 and IL-2. The results were calculated as means±SD. *, p<0.05, **, p<0.01, ***, p<0.001, ****, p<0.0001.

FIG. 8A is a graph showing interferon-γ (IFN-γ) production of CD3⁺CD56⁺ NKT cells isolated from healthy donor 2, 17 and 18 in response to a panel of AML cells in an enzyme-linked immunosorbent assay (ELISA). 3F (CD4⁺ NKT) and 4E (CD8⁺ NKT) clones were included as controls.

FIG. 8B is a graph showing interferon-γ (IFN-γ) production of CD3⁺CD56⁺ NKT cells isolated from healthy donor 20, 21 and 22 in response to a panel of AML cells in an enzyme-linked immunosorbent assay (ELISA).

FIG. 8C is a graph showing interferon-γ (IFN-γ) production of CD3⁺CD56⁺ NKT cells isolated from healthy donor 26, 27 and 29 in response to a panel of AML cells in an enzyme-linked immunosorbent assay (ELISA). 3F (CD4⁺ NKT) and 4E (CD8⁺ NKT) clones were included as controls.

FIG. 8D is a graph showing statistical analysis of interferon-γ (IFN-γ) production of CD3⁺CD56⁺ NKT cells isolated from 9 healthy donors in response to a panel of AML cells (HL60, KG-1, Molm-13, MV4:11, THP-1, U937) and B-cell malignancies (Daudi, Raji, Nalm6). The results were calculated as means±SD. *, p<0.05, **, p<0.01, ***, p<0.001, ****, p<0.0001.

FIG. 9A is a flow cytometric graph showing CD3⁺CD56⁺ NKT cell percentages after culture from 11 healthy donors and their CD4⁺, CD8⁺ and CD4⁻CD8⁻ percentages. Gating of CD3⁺CD56⁺ NKT cells was based on isotype antibody controls.

FIG. 9B is a flow cytometric graph showing CD3⁺CD56⁺ NKT cell percentages in PBMCs from 10 healthy donors and their purity after CD3⁺CD56⁺ NKT Cell Isolation. Gating of CD3⁺CD56⁺ NKT cells was based on isotype antibody controls. Gating of CD4⁺ and CD8⁺ cells was based on CD3⁺CD56⁺ populations.

FIG. 10A is a graph showing cytotoxicity of iNKT cells on AML cells pulsed with α-GalCer antigen in a luciferase-based killing assay at different E/T ratios.

FIG. 10B is a flow cytometric analysis plot showing CD3⁺ and iTCR⁺ marker expression of iNKT cells.

FIG. 10C is a flow cytometric graph showing AML cells expressing a variable amount of CD1d molecules on the cell surface. Gray shade dot line, isotype antibody; solid line, anti-human CD1d-PE.

FIG. 11A is a graph showing cytotoxicity of iNKT1, iNKT2 and iNKT12 cells (which were isolated from healthy blood donor 1, 2 and 12, stimulated with antigen αGalCer pulsed irradiated autologous, negative fractions of PBMCs and expanded with αGalCer pulsed irradiated allogeneic PBMCs) on AML cells pulsed with α-GalCer antigen in a luciferase-based killing assay at different E/T ratios.

FIG. 11B is a graph showing cytotoxicity of iNKT2 and iNKT12 cells on CD1d⁻ CML (K562), B-ALL (Nalm-6) and CD1d⁺ (MV4:11, U937) AML cells pulsed with α-GalCer antigen in a luciferase-based killing assay at different E/T ratios.

FIG. 11C is a graph showing blocking interferon-γ (IFN-γ) production of iNKT1 cells by an anti-human CD1d antibody (50 μg/mL, clone 42.1, BD Biosciences) in response to α-GalCer antigen stimulation in an enzyme-linked immunosorbent assay (ELISA).

FIG. 11D is a graph showing blocking cytotoxicity of iNKT1 and iNKT12 cells by anti-human CD1d antibodies (10 μg/mL, clone 42.1, BD Biosciences; clone 51.1, BioLegend) on AML (THP-1) cells pulsed with α-GalCer antigen in a luciferase-based killing assay at an E/T ratio of 5:1.

FIG. 11E is a graph showing blocking cytotoxicity of iNKT1 and iNKT12 cells by anti-human CD1d antibodies (40 μg/mL, clone 42.1, BD Biosciences; clone 51.1, BioLegend) on AML (THP-1) cells pulsed with α-GalCer antigen in a luciferase-based killing assay at E/T ratios of 5:1 and 30:1, respectively.

FIG. 11F is a graph showing blocking cytotoxicity of NKT30, NKT31 and 4E (CD8⁺ NKT) cells by an anti-human CD1d antibody (30 μg/mL, clone 51.1, BioLegend) on AML (Molm-13, U937) cells pulsed with α-GalCer antigen in a luciferase-based killing assay at E/T ratios of 15:1, 15:1 and 30:1, respectively.

FIG. 12A is a graph showing the effect of CD1d knock out (gRNA3+1, gRNA4+1 in U937-hffLucN cells, gRNA2 in MV4:11-hffLucN) AML cell lines on presentation of α-GalCer antigen to iNKT2 and iNKT12 cells in a luciferase-based killing assay at different E/T ratios. Wildtype U937-hfflucN and MV4:11-hfflucN cells were used as controls.

FIG. 12B is a graph showing the effect of CD1d knock out (gRNA1, gRNA2 in U937-hffLucN cells) AML cell lines on presentation of α-GalCer antigen to iNKT2 and iNKT12 cells in a luciferase-based killing assay at different E/T ratios.

FIG. 12C is a graph showing the effect of CD1d knock out (gRNA1, gRNA2, gRNA3+1, gRNA4+1 in U937-hffLucN cells and gRNA2 in MV4:11-hfflLucN) AML cell lines on presentation of α-GalCer antigen to iNKT2 and iNKT12 cells to release IFN-γ in an enzyme-linked immunosorbent assay (ELISA). Wildtype U937-hfflucN and MV4:11-hfflucN cells as well as CD1d⁻ Nalm-6-hffLucN cells were used as controls.

FIG. 12D is a graph showing the effect of CD1d knock out (gRNA1, gRNA2, gRNA3+1, gRNA4+1 in U937-hffLucN cells, gRNA2 in MV4:11-hffLucN) AML cell lines on presentation of α-GalCer antigen to iNKT1, iNKT2 and iNKT12 cells in a luciferase-based killing assay. iNKT1, E/T of 5:1; iNKT2, E/T of 15:1; iNKT12, E/T of 30:1, 10:1 and 3.3:1. Wildtype U937-hfflucN and MV4:11-hfflucN as well as CD1d⁻ Nalm6-hfflucN cells were used as positive and negative controls.

FIG. 12E is a graph showing the effect of CD1d knock out (gRNA1, gRNA2, gRNA3+1, gRNA4+1 in U937-hffLucN cells, gRNA2 in MV4:11-hffLucN) AML cell lines on presentation of α-GalCer antigen to CD3⁺CD56⁺ NKT2 and NKT17 cells to produce IFN-γ in an enzyme-linked immunosorbent assay (ELISA). 3F (CD4⁺ NKT) and 4E (CD8⁺ NKT) clones were included as controls.

FIG. 12F is a graph showing the effect of CD1d knock out (gRNA1, gRNA2, gRNA3+1, gRNA4+1 in U937-hffLucN cells, gRNA2 in MV4:11-hffLucN) AML cell lines on presentation of α-GalCer antigen to CD3⁺CD56⁺ NKT21 and NKT22 cells in a luciferase-based killing assay at different E/T ratios. Wildtype U937-hfflucN and MV4:11-hfflucN as well as CD1d⁻ Raji-hffLucN, Nalm6-hfflucN and K562-hffLucN cells were used as positive and negative controls.

FIG. 12G is a graph showing IFN-γ production of CD3⁺CD56⁺ NKT21 and NKT22 cells in recognition of CD1d knock out (gRNA1, gRNA2, gRNA3+1, gRNA4+1 in U937-hffLucN cells, gRNA2 in MV4:11-hffLucN) AML cell lines in an enzyme-linked immunosorbent assay (ELISA). Wildtype U937-hfflucN and MV4:11-hfflucN as well as CD1d⁻ Raji-hffLucN, Nalm6-hfflucN and K562-hffLucN cells were used as positive and negative controls.

FIG. 12H is a flow cytometric analysis plot showing CD1d cell surface expression in CD1d knock out (gRNA1, gRNA2, gRNA3+1, gRNA4+1 in U937-hffLucN cells, gRNA2 in MV4:11-hffLucN) AML cell lines. Wildtype U937-hfflucN and MV4:11-hfflucN as well as CD1d⁻ luciferase-expressing K562 and Nalm-6 cells were used as positive and negative controls.

FIG. 12I is a flow cytometric analysis plot showing CD1d cell surface expression in CD1d knock out (gRNA3+1 in U937-hffLucN cells) derived clones.

FIG. 12J is a flow cytometric analysis plot showing CD1d cell surface expression in CD1d knock out (gRNA4+1 in U937-hffLucN cells) derived clones.

FIG. 13 is a flow cytometric analysis plot showing the phenotypes of CD3⁺CD56⁺ NKT30 and NKT35 after culture. All phenotypic analysis was performed after gating of CD3⁺CD56⁺ NKT cells.

FIG. 14A is a flow cytometric analysis plot showing CAR expression in CD19 CAR lentivirus transduced 2A (DNT), 3F (CD4⁺ NKT) and 4E (CD8⁺ NKT) clones. Gray shade dot line, streptavidin PE only; solid line, biotin-protein-L plus streptavidin PE.

FIG. 14B is a graph showing cytotoxicity of CD19 CAR lentivirus transduced 2A (DNT), 3F (CD4⁺ NKT) and 4E (CD8⁺ NKT) clones on a cell panel in a luciferase-based killing assay.

### DETAILED DESCRIPTION OF THE DISCLOSURE

The present disclosure is based, in part, on the recognition that type III NKT cells (e.g., CD3⁺CD56⁺ type III NKT cells) may exhibit anti-cancer activity. Accordingly, in certain aspects, the present disclosure provides methods and compositions (e.g., pharmaceutical compositions) using type III NKT cells for treating or preventing a cancer in a subject in need thereof. In some embodiments, the type III NKT cells comprise or consist of CD3⁺CD56⁺ type III NKT cells. In some embodiments, the type III NKT cells comprise or consist of CD3⁺CD4⁺CD56⁺ cells. In some embodiments, the type III NKT cells comprise or consist of CD3⁺CD8⁺CD56⁺ cells. In some embodiments, the type III NKT cells comprise or consist of CD3⁺CD4⁻CD8⁻CD56⁺ cells. In some embodiments, the type III NKT cells comprise or consist of CD3⁺CD4⁺CD56⁺ cells, CD3⁺CD8⁺CD56⁺ cells and CD3⁺CD4⁻CD8⁻CD56⁺ cells. In some embodiments, treatment with the type III NKT cells of the present disclosure prevents or reduces undesirable off-target effects and/or toxicity (e.g., aplasia of normal myeloid cells) that may result from treatment with an alternate therapy.

In some embodiments, the exemplary type III NKT cells described herein are capable of treating and/or preventing a cancer (e.g., AML, e.g., refractory or relapsed AML). In some embodiments, the cancer is AML. In some embodiments, the cancer is refractory or relapsed AML. In some embodiments, the cancer is relapsed AML, e.g., after hematopoietic stem cell transplantation. In some embodiments, the cancer is a cancer that expresses an antigen targeted by the type III NKT cells and/or by a construct expressed by the type III NKT cells (e.g., a chimeric antigen receptor (CAR), a T cell receptor (TCR), or a T cell receptor mimic antibody (TCRm), or a combination thereof). In some embodiments, the cancer is a cancer that is resistant or refractory to treatment in the absence of the cells. Such exemplary cancers are described and exemplified herein.

In some embodiments, the exemplary type III NKT cells described herein are present and/or used in unmodified form. In some embodiments, the type III NKT cells can be used in unmodified form to treat and/or prevent a cancer. In some embodiments, unmodified type III NKT cells can be used as an off-the-shelf cancer therapy to treat and/or prevent a cancer. In some embodiments, the cancer is AML (e.g., refractory or relapsed AML).

In some embodiments, the exemplary type III NKT cells described herein are modified, and then present and/or used in modified form. In some embodiments, the type III NKT cells are modified, e.g., to express a CAR, a TCR, a TCRm, or a combination thereof. In some embodiments, the type III NKT cells can be used in modified form to treat and/or prevent a cancer. In some embodiments, modified type III NKT cells can be used as an off-the-shelf cancer therapy to treat and/or prevent a cancer. In some embodiments, the cancer is AML (e.g., refractory or relapsed AML). In some embodiments, the cancer is a cancer that expresses an antigen targeted by the type III NKT cells and/or by a construct expressed by the type III NKT cells (e.g., a CAR, a TCR, a TCRm, or a combination thereof).

In some embodiments, a cancer expressing a target antigen is a B-cell malignancy, leukemia, lymphoma, myeloma, or melanoma. In some embodiments, a cancer expressing a target antigen is AML, B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma. In some embodiments, a cancer expressing a target antigen is AML. In some embodiments, a cancer expressing a target antigen is resistant or refractory to treatment in the absence of the cells.

In some embodiments, the present disclosure provides a method of treating or preventing a cancer in a subject in need thereof, comprising administering to the subject a therapeutically effective amount of type III NKT cells (e.g., CD3⁺CD56⁺ type III NKT cells), or a pharmaceutical composition comprising a therapeutically effective amount of type III NKT cells (e.g., CD3⁺CD56⁺ type III NKT cells). In some embodiments, the pharmaceutical composition further comprises at least one pharmaceutically acceptable carrier.

In some embodiments, the present disclosure provides a method of preparing a therapy for treating or preventing a cancer in a subject in need thereof, comprising: (a) isolating one or more type III NKT cells (e.g., CD3⁺CD56⁺ type III NKT cells) from a biological sample; and (b) culturing the one or more cells in a growth medium to produce an expanded cell population. In some embodiments, the method further comprises modifying the one or more cells to express a CAR, TCR, or TCRm. In some embodiments, the modifying comprises introducing one or more polynucleotides encoding the CAR, TCR, or TCRm into the one or more cells. In some embodiments, introducing one or more polynucleotides comprises electroporation, transduction, and/or transfection. In some embodiments, the one or more polynucleotides comprise mRNA and/or DNA. In some embodiments, the DNA comprises transposon DNA. In some embodiments, the one or more polynucleotides comprise one or more vectors. In some embodiments, the one or more vectors comprise one or more viral vectors. In some embodiments, the one or more vectors comprise one or more lentiviral vectors or γ-retroviral vectors.

In some embodiments, the present disclosure provides a method of treating or preventing a cancer in a subject in need thereof, comprising administering to the subject a therapeutically effective amount of an expanded cell population, or a pharmaceutical composition comprising a therapeutically effective amount of an expanded cell population. In some embodiments, the expanded cell population comprises any exemplary expanded cell population described herein and/or prepared by a method described herein. In some embodiments, the expanded cell population comprises an expanded type III NKT cell population (e.g., an expanded CD3⁺CD56⁺ type III NKT cell population). In some embodiments, the expanded cell population comprises an expanded CD3⁺CD56⁺ type III NKT cell population. In some embodiments, the pharmaceutical composition further comprises at least one pharmaceutically acceptable carrier.

In some embodiments of the methods described herein, a cancer is a solid tumor or a hematological malignancy. In some embodiments, the cancer is a B-cell malignancy, leukemia, lymphoma, myeloma, or melanoma. In some embodiments, the cancer is acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma. In some embodiments, the cancer is AML. In some embodiments, the cancer is resistant or refractory to treatment in the absence of the cells.

In some embodiments of the methods described herein, type III NKT cells are CD3⁺CD56⁺ type III NKT cells. In some embodiments, the type III NKT cells are CD3⁺CD4⁺CD56⁺ cells. In some embodiments, the type III NKT cells are CD3⁺CD8⁺CD56⁺ cells. In some embodiments, the type III NKT cells are CD3⁺CD4⁻CD8⁻CD56⁺ cells. In some embodiments, the type III NKT cells are CD3⁺CD4⁺CD56⁺ cells, CD3⁺CD8⁺CD56⁺ cells, and CD3⁺CD4⁻CD8⁻CD56⁺ cells. In some embodiments, the type III NKT cells are isolated from a biological sample. In some embodiments, the biological sample is from the subject (e.g., a cancer patient). In some embodiments, the biological sample is from a donor (e.g., a healthy donor). In some embodiments, the biological sample comprises blood, bone marrow, lymph node tissue, spleen tissue, tumor tissue, one or more induced pluripotent stem cells, and/or one or more peripheral blood mononuclear cells. In some embodiments, the blood comprises peripheral blood and/or umbilical cord blood. In some embodiments, the type III NKT cells are isolated from one or more peripheral blood mononuclear cells.

In some embodiments of the methods described herein, type III NKT cells are modified to express a chimeric antigen receptor (CAR). In some embodiments, the cells comprise one or more polynucleotides encoding the CAR. In some embodiments, the CAR comprises at least an antigen binding domain, a transmembrane domain, and an intracellular signaling domain.

In some embodiments, an antigen binding domain of a CAR is capable of binding to CD19, IGF1R, ROR1, BCMA, CD123, CD33, CD38, CD138, CLL-1, LILRB4, GD2, CD20, CD22, CD30, MSLN, EGFRvIII, EGFR, HER2, MUC1, EPCAM, PSMA, SLAMF7, GPC3, or PD-L1. In some embodiments, the antigen binding domain is capable of binding to CD19, IGF1R, or ROR1.

In some embodiments, the antigen binding domain and/or CAR is capable of binding to CD19. In some embodiments, the antigen binding domain and/or CAR is capable of binding to IGF1R. In some embodiments, the antigen binding domain and/or CAR is capable of binding to ROR1. In some embodiments, the antigen binding domain and/or CAR is capable of binding to BCMA. In some embodiments, the antigen binding domain and/or CAR is capable of binding to CD123. In some embodiments, the antigen binding domain and/or CAR is capable of binding to CD33. In some embodiments, the antigen binding domain and/or CAR is capable of binding to CD38. In some embodiments, the antigen binding domain and/or CAR is capable of binding to CD138. In some embodiments, the antigen binding domain and/or CAR is capable of binding to CLL-1. In some embodiments, the antigen binding domain and/or CAR is capable of binding to LILRB4. In some embodiments, the antigen binding domain and/or CAR is capable of binding to GD2. In some embodiments, the antigen binding domain and/or CAR is capable of binding to CD20. In some embodiments, the antigen binding domain and/or CAR is capable of binding to CD22. In some embodiments, the antigen binding domain and/or CAR is capable of binding to CD30. In some embodiments, the antigen binding domain and/or CAR is capable of binding to MSLN. In some embodiments, the antigen binding domain and/or CAR is capable of binding to EGFRvIII. In some embodiments, the antigen binding domain and/or CAR is capable of binding to EGFR. In some embodiments, the antigen binding domain and/or CAR is capable of binding to HER2. In some embodiments, the antigen binding domain and/or CAR is capable of binding to MUC1. In some embodiments, the antigen binding domain and/or CAR is capable of binding to EPCAM. In some embodiments, the antigen binding domain and/or CAR is capable of binding to PSMA. In some embodiments, the antigen binding domain and/or CAR is capable of binding to SLAMF7. In some embodiments, the antigen binding domain and/or CAR is capable of binding to GPC3. In some embodiments, the antigen binding domain and/or CAR is capable of binding to PD-L1.

In some embodiments, the antigen binding domain and/or CAR is capable of binding to CD19 and the cancer is a B-cell malignancy (e.g., B-cell precursor acute lymphoblastic leukemia (B-ALL), non-Hodgkin lymphoma (NHL), or chronic lymphocytic leukemia (CLL)). In some embodiments, the antigen binding domain and/or CAR is capable of binding to CD19 and the cancer is B-ALL, NHL, or CLL. In some embodiments, the antigen binding domain and/or CAR is capable of binding to ROR1 and the cancer is Ewing sarcoma, osteosarcoma, fibrosarcoma, rhabdomyosarcoma, chronic lymphocytic leukemia, mantle cell carcinoma, breast cancer, lung adenocarcinoma, melanoma, or ovarian cancer. In some embodiments, the antigen binding domain and/or CAR is capable of binding to BCMA and the cancer is multiple myeloma. In some embodiments, the antigen binding domain and/or CAR is capable of binding to CD123, CD33, CD38, CD138, CLL-1, or LILRB4 and the cancer is AML. In some embodiments, the antigen binding domain and/or CAR is capable of binding to EGFRvIII or EGFR and the cancer is glioblastoma. In some embodiments, the antigen binding domain and/or CAR is capable of binding to GPC3 and the cancer is hepatocellular carcinoma.

In some embodiments, an antigen binding domain of a CAR comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody. In some embodiments, the antigen binding fragment comprises a single chain variable fragment (scFv).

In some embodiments, an intracellular signaling domain of a CAR comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1BB, CD28, CD27, CD134 (OX40), ICOS, DAP10, or DAP12.

In some embodiments of the methods described herein, type III NKT cells are modified to express a T cell receptor (TCR). In some embodiments, the cells comprise one or more polynucleotides encoding the TCR. In some embodiments, the TCR comprises at least an alpha chain and a beta chain. In some embodiments, the alpha chain and/or the beta chain is capable of binding to an antigen. In some embodiments, the antigen is an intracellular antigen. In some embodiments, the antigen is NY-ESO-1, WT1, or MAGE-A3.

In some embodiments, an alpha chain and/or a beta chain of a TCR is capable of binding to NY-ESO-1, WT1, or MAGE-A3. In some embodiments, the alpha chain, beta chain, and/or TCR is capable of binding to NY-ESO-1. In some embodiments, the alpha chain, beta chain, and/or TCR is capable of binding to WT1. In some embodiments, the alpha chain, beta chain, and/or TCR is capable of binding to MAGE-A3.

In some embodiments, the antigen is NY-ESO-1. In some embodiments, the alpha chain, beta chain, and/or TCR is capable of binding to NY-ESO-1 and the cancer is neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, or breast cancer. In some embodiments, the antigen is WT1. In some embodiments, the alpha chain, beta chain, and/or TCR is capable of binding to WT1 and the cancer is AML.

In some embodiments of the methods described herein, type III NKT cells are modified to express a T cell receptor mimic antibody (TCRm). In some embodiments, the cells comprise one or more polynucleotides encoding the TCRm. In some embodiments, the TCRm comprises at least an antigen binding domain, a transmembrane domain, and an intracellular signaling domain.

In some embodiments, an antigen binding domain of a TCRm is capable of binding to a composite antigen. In some embodiments, an composite antigen comprises a peptide and a human leukocyte antigen (HLA) molecule.

In some embodiments, an HLA molecule is a class I HLA molecule. In some embodiments, a HLA molecule is a class II HLA molecule.

In some embodiments, a peptide comprises an alpha fetoprotein (AFP) peptide. In some embodiments, the composite antigen comprises an AFP peptide and a HLA-A2 molecule. In some embodiments, the cancer is hepatocellular carcinoma.

In some embodiments, a peptide comprises a preferentially expressed antigen in melanoma (PRAME) peptide. In some embodiments, the PRAME peptide comprises an amino acid sequence of ALYVDSLFFL (SEQ ID NO: 41). In some embodiments, the composite antigen comprises a PRAME peptide and a HLA-A*0201 molecule. In some embodiments, the cancer is B-ALL, AML, multiple myeloma, T cell lymphoma, melanoma, non-small cell lung cancer, colon adenocarcinoma, or breast adenocarcinoma.

In some embodiments, a peptide comprises a WT1 peptide. In some embodiments, the composite antigen comprises a WT1 peptide and a HLA-A2 molecule. In some embodiments, the cancer is AML.

In some embodiments, an antigen binding domain of a TCRm comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody. In some embodiments, the antigen binding fragment comprises a single chain variable fragment (scFv).

In some embodiments, an intracellular signaling domain of a TCRm comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1 BB, CD28, CD27, CD134 (OX40), ICOS, DAP10, or DAP12.

In some embodiments of the methods described herein, type III NKT cells are further modified to comprise an exogenous cytokine, growth factor, antibody or antigen binding fragment, or any combination thereof. In some embodiments, the antibody or antigen binding fragment comprises a bispecific T cell engager (BiTE).

Further provided herein, in some embodiments, are pharmaceutical compositions comprising isolated type III NKT cells (e.g., CD3⁺CD56⁺ type III NKT cells). In some embodiments, the present disclosure provides a pharmaceutical composition for treating or preventing a cancer in a subject in need thereof comprising isolated type III NKT cells (e.g., CD3⁺CD56⁺ type III NKT cells) and at least one pharmaceutically acceptable carrier. In some embodiments, the type III NKT cells in the pharmaceutical composition are CD3⁺CD56⁺ type III NKT cells (e.g., CD3⁺CD4⁺CD56⁺ cells, CD3⁺CD8⁺CD56⁺ cells, CD3⁺CD4⁻CD8⁻CD56⁺ cells or a mixture thereof). In some embodiments, the type III NKT cells in the pharmaceutical composition are CD3⁺CD4⁺CD56⁺ cells. In some embodiments, the type III NKT cells in the pharmaceutical composition are CD3⁺CD8⁺CD56⁺ cells. In some embodiments, the type III NKT cells in the pharmaceutical composition are CD3⁺CD4⁻CD8⁻CD56⁺ cells. In some embodiments, the type III NKT cells in the pharmaceutical composition are CD3⁺CD4⁺CD56⁺ cells, CD3⁺CD8⁺CD56⁺ cells, and CD3⁺CD4⁻CD8⁻CD56⁺ cells.

Also provided herein, in some embodiments, are therapeutic uses for type III NKT cells (e.g., CD3⁺CD56⁺ type III NKT cells), or pharmaceutical compositions comprising the same.

An exemplary embodiment is isolated type III NKT cells (e.g., CD3⁺CD56⁺ type III NKT cells) for use in treating or preventing a cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated type III NKT cells (e.g., CD3⁺CD56⁺ type III NKT cells) in treating or preventing a cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated type III NKT cells (e.g., CD3⁺CD56⁺ type III NKT cells) in the manufacture of a medicament for treating or preventing a cancer in a subject in need thereof. In some embodiments, the medicament comprises a therapeutically effective amount of the cells, or a pharmaceutical composition comprising a therapeutically effective amount of the cells and at least one pharmaceutically acceptable carrier.

In some embodiments of the cells and uses described herein, a cancer is a solid tumor or a hematological malignancy. In some embodiments, the cancer is a B-cell malignancy, leukemia, lymphoma, myeloma, or melanoma. In some embodiments, the cancer is acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma. In some embodiments, the cancer is AML. In some embodiments, the cancer is resistant or refractory to treatment in the absence of the cells.

In some embodiments of the cells and uses described herein, type III NKT cells are CD3⁺CD56⁺ type III NKT cells. In some embodiments, the type III NKT cells are CD3⁺CD4⁺CD56⁺ cells. In some embodiments, the type III NKT cells are CD3⁺CD8⁺CD56⁺ cells. In some embodiments, the type III NKT cells are CD3⁺CD4⁻CD8⁻CD56⁺ cells. In some embodiments, the type III NKT cells are CD3⁺CD4⁺CD56⁺ cells, CD3⁺CD8⁺CD56⁺ cells, and CD3⁺CD4⁻CD8⁻CD56⁺ cells. In some embodiments, the type III NKT cells are isolated from a biological sample. In some embodiments, the biological sample is from the subject (e.g., a cancer patient). In some embodiments, the biological sample is from a donor (e.g., a healthy donor). In some embodiments, the biological sample comprises blood, bone marrow, lymph node tissue, spleen tissue, tumor tissue, one or more induced pluripotent stem cells, and/or one or more peripheral blood mononuclear cells. In some embodiments, the blood comprises peripheral blood and/or umbilical cord blood. In some embodiments, the type III NKT cells are isolated from one or more peripheral blood mononuclear cells.

In some embodiments of the cells and uses described herein, type III NKT cells are modified to express a chimeric antigen receptor (CAR). In some embodiments, the cells comprise one or more polynucleotides encoding the CAR. In some embodiments, the CAR comprises at least an antigen binding domain, a transmembrane domain, and an intracellular signaling domain.

In some embodiments, an antigen binding domain of a CAR is capable of binding to CD19, IGF1R, ROR1, BCMA, CD123, CD33, CD38, CD138, CLL-1, LILRB4, GD2, CD20, CD22, CD30, MSLN, EGFRvIII, EGFR, HER2, MUC1, EPCAM, PSMA, SLAMF7, GPC3, or PD-L1. In some embodiments, the antigen binding domain is capable of binding to CD19, IGF1R, or ROR1.

In some embodiments, the antigen binding domain and/or CAR is capable of binding to CD19. In some embodiments, the antigen binding domain and/or CAR is capable of binding to IGF1R. In some embodiments, the antigen binding domain and/or CAR is capable of binding to ROR1. In some embodiments, the antigen binding domain and/or CAR is capable of binding to BCMA. In some embodiments, the antigen binding domain and/or CAR is capable of binding to CD123. In some embodiments, the antigen binding domain and/or CAR is capable of binding to CD33. In some embodiments, the antigen binding domain and/or CAR is capable of binding to CD38. In some embodiments, the antigen binding domain and/or CAR is capable of binding to CD138. In some embodiments, the antigen binding domain and/or CAR is capable of binding to CLL-1. In some embodiments, the antigen binding domain and/or CAR is capable of binding to LILRB4. In some embodiments, the antigen binding domain and/or CAR is capable of binding to GD2. In some embodiments, the antigen binding domain and/or CAR is capable of binding to CD20. In some embodiments, the antigen binding domain and/or CAR is capable of binding to CD22. In some embodiments, the antigen binding domain and/or CAR is capable of binding to CD30. In some embodiments, the antigen binding domain and/or CAR is capable of binding to MSLN. In some embodiments, the antigen binding domain and/or CAR is capable of binding to EGFRvIII. In some embodiments, the antigen binding domain and/or CAR is capable of binding to EGFR. In some embodiments, the antigen binding domain and/or CAR is capable of binding to HER2. In some embodiments, the antigen binding domain and/or CAR is capable of binding to MUC1. In some embodiments, the antigen binding domain and/or CAR is capable of binding to EPCAM. In some embodiments, the antigen binding domain and/or CAR is capable of binding to PSMA. In some embodiments, the antigen binding domain and/or CAR is capable of binding to SLAMF7. In some embodiments, the antigen binding domain and/or CAR is capable of binding to GPC3. In some embodiments, the antigen binding domain and/or CAR is capable of binding to PD-L1.

In some embodiments, the antigen binding domain and/or CAR is capable of binding to CD19 and the cancer is a B-cell malignancy (e.g., B-cell precursor acute lymphoblastic leukemia (B-ALL), non-Hodgkin lymphoma (NHL), or chronic lymphocytic leukemia (CLL)). In some embodiments, the antigen binding domain and/or CAR is capable of binding to CD19 and the cancer is B-ALL, NHL, or CLL. In some embodiments, the antigen binding domain and/or CAR is capable of binding to ROR1 and the cancer is Ewing sarcoma, osteosarcoma, fibrosarcoma, rhabdomyosarcoma, chronic lymphocytic leukemia, mantle cell carcinoma, breast cancer, lung adenocarcinoma, melanoma, or ovarian cancer. In some embodiments, the antigen binding domain and/or CAR is capable of binding to BCMA and the cancer is multiple myeloma. In some embodiments, the antigen binding domain and/or CAR is capable of binding to CD123, CD33, CD38, CD138, CLL-1, or LILRB4 and the cancer is AML. In some embodiments, the antigen binding domain and/or CAR is capable of binding to EGFRvIII or EGFR and the cancer is glioblastoma. In some embodiments, the antigen binding domain and/or CAR is capable of binding to GPC3 and the cancer is hepatocellular carcinoma.

In some embodiments, an antigen binding domain of a CAR comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody. In some embodiments, the antigen binding fragment comprises a single chain variable fragment (scFv).

In some embodiments, an intracellular signaling domain of a CAR comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1BB, CD28, CD27, CD134 (OX40), ICOS, DAP10, or DAP12.

In some embodiments of the cells and uses described herein, type III NKT cells are modified to express a T cell receptor (TCR). In some embodiments, the cells comprise one or more polynucleotides encoding the TCR. In some embodiments, the TCR comprises at least an alpha chain and a beta chain. In some embodiments, the alpha chain and/or the beta chain is capable of binding to an antigen. In some embodiments, the antigen is an intracellular antigen. In some embodiments, the antigen is NY-ESO-1, WT1, or MAGE-A3.

In some embodiments, an alpha chain and/or a beta chain of a TCR is capable of binding to NY-ESO-1, WT1, or MAGE-A3. In some embodiments, the alpha chain, beta chain, and/or TCR is capable of binding to NY-ESO-1. In some embodiments, the alpha chain, beta chain, and/or TCR is capable of binding to WT1. In some embodiments, the alpha chain, beta chain, and/or TCR is capable of binding to MAGE-A3.

In some embodiments, the antigen is NY-ESO-1. In some embodiments, the alpha chain, beta chain, and/or TCR is capable of binding to NY-ESO-1 and the cancer is neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, or breast cancer. In some embodiments, the antigen is WT1. In some embodiments, the alpha chain, beta chain, and/or TCR is capable of binding to WT1 and the cancer is AML.

In some embodiments of the cells and uses described herein, type III NKT cells are modified to express a T cell receptor mimic antibody (TCRm). In some embodiments, the cells comprise one or more polynucleotides encoding the TCRm. In some embodiments, the TCRm comprises at least an antigen binding domain, a transmembrane domain, and an intracellular signaling domain.

In some embodiments, an antigen binding domain of a TCRm is capable of binding to a composite antigen. In some embodiments, an composite antigen comprises a peptide and a human leukocyte antigen (HLA) molecule.

In some embodiments, an HLA molecule is a class I HLA molecule. In some embodiments, a HLA molecule is a class II HLA molecule.

In some embodiments, a peptide comprises an alpha fetoprotein (AFP) peptide. In some embodiments, the composite antigen comprises an AFP peptide and a HLA-A2 molecule. In some embodiments, the cancer is hepatocellular carcinoma.

In some embodiments, a peptide comprises a preferentially expressed antigen in melanoma (PRAME) peptide. In some embodiments, the PRAME peptide comprises an amino acid sequence of ALYVDSLFFL (SEQ ID NO: 41). In some embodiments, the composite antigen comprises a PRAME peptide and a HLA-A*0201 molecule. In some embodiments, the cancer is B-ALL, AML, multiple myeloma, T cell lymphoma, melanoma, non-small cell lung cancer, colon adenocarcinoma, or breast adenocarcinoma.

In some embodiments, a peptide comprises a WT1 peptide. In some embodiments, the composite antigen comprises a WT1 peptide and a HLA-A2 molecule. In some embodiments, the cancer is AML.

In some embodiments, an antigen binding domain of a TCRm comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody. In some embodiments, the antigen binding fragment comprises a single chain variable fragment (scFv).

In some embodiments, an intracellular signaling domain of a TCRm comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1 BB, CD28, CD27, CD134 (OX40), ICOS, DAP10, or DAP12.

In some embodiments of the cells and uses described herein, type III NKT cells are further modified to comprise an exogenous cytokine, growth factor, antibody or antigen binding fragment, or any combination thereof. In some embodiments, the antibody or antigen binding fragment comprises a bispecific T cell engager (BiTE).

### Certain Illustrative Embodiments

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein are also incorporated by reference in their entirety. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise. All ranges include the endpoints and all points in between unless the context indicates otherwise.

The term “about” or “approximately,” as used herein in the context of numerical values and ranges, refers to values or ranges that approximate or are close to the recited values or ranges such that the embodiment may perform as intended, as is apparent to the skilled person from the teachings contained herein. This is due, at least in part, to the varying properties of nucleic acid compositions, age, race, gender, anatomical and physiological variations and the inexactitude of biological systems. Thus, these terms encompass values beyond those resulting from systematic error. In some embodiments, “about” or “approximately” means plus or minus 10% of a numerical amount.

### NKT Cells

In certain aspects, the present disclosure provides type III natural killer T (NKT) cells (e.g., CD3⁺CD56⁺ type III NKT cells), as well as methods and compositions using the type III NKT cells described herein.

As used herein, the term “natural killer T cell” or “NKT cell,” refers to a T cell or a T cell population that exhibits characteristics of both conventional T cells and natural killer (NK) cells. For instance, in some embodiments, an NKT cell is a mature lymphocyte that bears both T and NK cell receptors. In some embodiments, an NKT cell arises in the thymus from CD4⁺CD8⁺ cortical thymocytes that have undergone T cell receptor (TCR) gene rearrangement.

There are two classifications of NKT cells in literatures. One classic classification of NKT cells refers NKT cells to a subgroup of unconventional T cells that recognize lipid antigens presented by MHC class I-like CD1d molecules and divides NKT cells into type I and type II NKT cells (Godfrey et al. Nat Immunol. 2010; 11(3):197-206; Dhodapkar and Kumar. J Immunol. 2017; 198(3):1015-21; Godfrey et al. Immunity. 2018; 48(3):453-73). Another classification of NKT cells includes type I, type II and type III NKT (NKT-like) cells (Godfrey et al. Nat Rev Immunol. 2004; 4(3):231-237; Farr et al. Proc Natl Acad Sci USA. 2014; 111(35):12841-6).

As used herein, the term “type I NKT cell” or “invariant NKT cell” or “iNKT cell” refers to an NKT cell or an NKT cell population that expresses an invariant or semi-invariant TCR repertoire and binds to the glycosphingolipid α-galactosylceramide (α-GalCer) in association with MHC class I-like CD1d molecules. In some embodiments, a type I NKT cell expresses an invariant TCRα-chain and a limited number of non-invariant TCRβ-chains. In some embodiments, a type I NKT cell expresses a semi-invariant Va chain (e.g., Vα14-Jα18 TCR in mice, and Vα24-Jα18 in humans), paired with a limited repertoire of Vβ-chains (e.g., Vβ8.2, Vβ7, and Vβ2 in mice, and Vβ11 in humans). In some embodiments, a type I NKT cell recognizes the glycosphingolipid α-galactosylceramide (α-GalCer) or a synthetic analog thereof when presented by MHC class I-like CD1d molecules.

As used herein, the term “type II NKT cell” or “diverse NKT cell” refers to an NKT cell or an NKT cell population that expresses a variant TCR repertoire and does not bind to the glycosphingolipid α-galactosylceramide (α-GalCer). Instead, in some embodiments, a type II NKT cell recognizes at least one non-α-GalCer molecule (e.g., sulfatide) when presented by MHC class I-like CD1d molecules. In some embodiments, a type II NKT cell may produce IL-4 and/or IFN-γ.

As used herein, the term “type III NKT cell” refers to CD1d-unrestricted or CD1d-independent NKT cells that exhibits characteristics of both conventional T cells and natural killer (NK) cells, e.g. CD3⁺CD56⁺ in humans or NK1.1⁺CD3⁺ in mice.

It is worthnoting that we claimed CD3⁺CD56⁺ NKT cells as type II NKT cells in the provisional application since our data showed that CD3⁺CD56⁺ NKT cells recognized all CD1d⁺ AML cell lines tested but not CD1d⁻ B-ALL and lymphoma cells. Moreover, it is unclear whether CD3⁺CD56⁺ cells are CD1d-restricted (Krijgsman et al. Front Immunol. 2018; 9:367) although CD1d-unrestricted NKT cells can be present in CD1d deficient mice (Farr et al. Proc Natl Acad Sci USA. 2014; 111(35):12841-6). With our definitive data of CRISPR-mediated CD1d knockout AML cells in this PCT application, CD3⁺CD56⁺ NKT cells should be renamed as type III NKT cells. Methods and compositions using CD3⁺CD56⁺ NKT cells in the provisional application remain unchanged in this PCT application.

In some embodiments, an NKT cell (e.g., a type III NKT cell) is a single cell. In some embodiments, an NKT cell (e.g., a type III NKT cell) is a homogenous cell population. In some embodiments, an NKT cell (e.g., a type III NKT cell) is a heterogenous cell population. In some embodiments, an NKT cell (e.g., a type III NKT cell) causes, stimulates, and/or contributes to the production of at least one cytokine (e.g., IL-4 and/or IFN-γ). In some embodiments, an NKT cell (e.g., a type III NKT cell) is cytotoxic. In some embodiments, an NKT cell (e.g., a type III NKT cell) may display cytotoxicity against various cells, including cancer cells or cell lines (e.g., AML cells or cell lines), as described and exemplified herein. In some embodiments, an NKT cell is a type I or a type II NKT cell. In some embodiments, an NKT cell is a type III NKT cell.

In some embodiments, type III NKT cells of the present disclosure may express any number or combination of cell surface markers. For instance, in some embodiments, type III NKT cells may express CD3 and CD56 on the cell surface. In some embodiments, the CD3 and CD56 cell surface markers may be expressed on their own, or in combination with one or more additional cell surface markers (e.g., CD4⁺, CD8⁺, CD4⁻CD8⁻ etc.).

As used herein, the term “CD3⁺CD56⁺” may be used to describe any cell (e.g., any type III NKT cell) that expresses at least CD3 and CD56 on its cell surface. In some embodiments, the type III NKT cells used in the methods and compositions described herein are CD3⁺CD56⁺ cells. In some embodiments, CD3⁺CD56⁺ cells may also express one or more additional cell surface markers (e.g., CD4 or CD8).

As used herein, the term “CD3⁺CD4⁺CD56⁺” may be used to describe any cell (e.g., any type III NKT cell) that expresses at least CD3, CD4, and CD56 on its cell surface. In some embodiments, the type III NKT cells used in the methods and compositions described herein are CD3⁺CD4⁺CD56⁺ cells. In some embodiments, the type III NKT cells used in the methods and compositions described herein are a mixture of CD3⁺CD4⁺CD56⁺ cells and at least one additional cell type (e.g., CD3⁺CD8⁺CD56⁺ cells, CD3⁺CD4⁻CD8⁻CD56⁺). In some embodiments, CD3⁺CD4⁺CD56⁺ cells may also express one or more additional cell surface markers.

As used herein, the term “CD3⁺CD8⁺CD56⁺” may be used to describe any cell (e.g., any type III NKT cell) that expresses at least CD3, CD8, and CD56 on its cell surface. In some embodiments, the type III NKT cells used in the methods and compositions described herein are CD3⁺CD8⁺CD56⁺ cells. In some embodiments, the type III NKT cells used in the methods and compositions described herein are a mixture of CD3⁺CD8⁺CD56⁺ cells and at least one additional cell type (e.g., CD3⁺CD4⁺CD56⁺ cells, CD3⁺CD4⁻CD8⁻CD56⁺ cells). In some embodiments, CD3⁺CD8⁺CD56⁺ cells may also express one or more additional cell surface markers.

As used herein, the term “CD3⁺CD4⁻CD8⁻CD56⁺” may be used to describe any cell (e.g., any type III NKT cell) that expresses at least CD3, CD56, and CD4⁻CD8⁻ on its cell surface. In some embodiments, the type III NKT cells used in the methods and compositions described herein are CD3⁺CD4⁻CD8⁻CD56⁺ cells. In some embodiments, the type III NKT cells used in the methods and compositions described herein are a mixture of CD3⁺CD4⁻CD8⁻CD56⁺ cells and at least one additional cell type (e.g., CD3⁺CD4⁺CD56⁺ cells, CD3⁺CD8⁺CD56⁺ cells). In some embodiments, CD3⁺CD4⁻CD8⁻CD56⁺ cells may also express one or more additional cell surface markers.

In some embodiments, type III NKT cells of the present disclosure (e.g., CD3⁺CD56⁺ type III NKT cells) may be obtained or isolated from a biological sample. In some embodiments, type III NKT cells of the present disclosure may be obtained or isolated from one or more peripheral blood mononuclear cells.

In some embodiments, a biological sample is from a human (e.g., a fetal, neonatal, child, or adult human). In some embodiments, the biological sample is from a non-human animal. Non-human animals include all vertebrates (e.g., mammals and non-mammals) such as mice, rats, rabbits, dogs, monkeys, and pigs. In some embodiments, the biological sample is from a subject in need of treatment (e.g., a cancer patient, e.g., an AML patient). In some embodiments, the biological sample is from a donor (e.g., a healthy donor). In some embodiments, the biological sample comprises blood (e.g., peripheral blood and/or umbilical cord blood), bone marrow, lymph node tissue, spleen tissue, tumor tissue, one or more induced pluripotent stem cells, and/or one or more peripheral blood mononuclear cells. In some embodiments, the biological sample and/or blood comprises peripheral blood and/or umbilical cord blood. In some embodiments, the biological sample and/or blood is collected (e.g., from a subject or donor) by apheresis and/or leukapheresis.

In some embodiments, type III NKT cells of the present disclosure (e.g., CD3⁺CD56⁺ type III NKT cells) may be isolated, e.g., from a human biological sample. In some embodiments, the type III NKT cells are isolated type III NKT cells.

As used herein, the term “isolated” refers to a material that is removed from its source environment (e.g., the natural environment if it is naturally-occurring). For example, a naturally-occurring polynucleotide, polypeptide, or cell present in a living organism is not isolated, but the same polynucleotide, polypeptide, or cell separated from some or all of the coexisting materials in the living organism, is isolated.

An “isolated cell,” as used herein, refers to a cell or cell population (e.g., a type III NKT cell or cell population) that has been identified and separated from one or more (e.g., the majority) of the components of its source environment (e.g., from the components of a cell culture or a biological sample). In some embodiments, the separation is performed such that it sufficiently removes components that may otherwise interfere with the suitability of the cell for the desired applications (e.g., for therapeutic use of a type III NKT cell or cell population). In some embodiments, the separation is performed such that it sufficiently separates cells expressing a particular marker or set of markers (e.g., CD3 and CD56) from cells expressing an alternate marker or set of markers. Methods for isolating cells are known in the art and include, without limitation, separation by positive and/or negative selection techniques, or by cell sorting, for example, using antibody-conjugated microbeads, using flow cytometry with a cocktail of monoclonal antibodies directed to cell surface markers, etc. Exemplary isolation and separation techniques are described and exemplified herein.

In some embodiments, type III NKT cells of the present disclosure (e.g., CD3⁺CD56⁺ type III NKT cells) may be isolated or separated via affinity-based separation methods. Exemplary techniques for affinity separation may include, in some embodiments, magnetic separation (e.g., using antibody-coated magnetic beads), affinity chromatography, cytotoxic agents joined to a monoclonal antibody or use in conjunction with a monoclonal antibody (e.g., complement and cytotoxins), and “panning” with an antibody attached to a solid matrix (e.g., a plate), or any other suitable technique. In some embodiments, separation techniques may also include the use of fluorescence activated cell sorters, which can have varying degrees of sophistication, such as multiple color channels, low angle and obtuse light scattering detecting channels, impedance channels, etc. It is to be understood that any technique that enables isolation or separation of type III NKT cells (e.g., CD3⁺CD56⁺ type III NKT cells) may be employed.

In some embodiments, affinity reagents employed in various isolation or separation methods may be specific receptors or ligands for cell surface markers on the type III NKT cells. In some embodiments, antibodies may be conjugated to a label, which may, in some embodiments, be used for isolation or separation. Labels may include, in some embodiments, magnetic beads (e.g., which may allow for direct separation), biotin (e.g., which may be removed with avidin or streptavidin bound to, e.g., a support), fluorochromes (e.g., which may be used with a fluorescence activated cell sorter, e.g., phycoerythrin, fluorescein, Texas red, or a combination thereof), or the like.

In some embodiments, cell separations utilizing antibodies may comprise the addition of an antibody to a suspension of cells, e.g., for a period of time sufficient to bind available cell surface markers. The incubation may be for a varied period of time. For example, in some embodiments, the incubation may be for about 2 minutes, about 5 minutes, about 15 minutes, about 30 minutes, about 45 minutes, about 60 minutes, about 90 minutes, or longer. Any length of time which results in specific labeling with the antibody, with minimal non-specific binding, may be considered envisioned for this aspect of the disclosure.

In some embodiments, staining intensity of type III NKT cells can be monitored by flow cytometry, for example, where lasers detect quantitative levels of a fluorochrome (which may be proportional to the amount of cell surface antigen bound by antibodies). Flow cytometry, or FACS, can also be used, in some embodiments, to separate cell populations based on the intensity of antibody staining, as well as other parameters such as cell size and light scatter.

In some embodiments, type III NKT cells are separated based on their expression of at least one cell surface marker. The separated cells may be collected in any appropriate medium that maintains cell viability. In some embodiments, a culture containing the cells may contain serum, cytokines, or growth factors to which the cells are responsive.

In some embodiments, a cytokine or growth factor may promote cell survival, growth, function, or a combination thereof. Cytokines and growth factors may include, in some embodiments, polypeptides and non-polypeptide factors.

In some embodiments, type III NKT cells of the present disclosure (e.g., CD3⁺CD56⁺ type III NKT cells) may be modified, e.g., to express a construct capable of binding to a target antigen. In some embodiments, type III NKT cells of the present disclosure (e.g., CD3⁺CD56⁺ type III NKT cells) are modified to express a chimeric antigen receptor (CAR), a T cell receptor (TCR), a T cell receptor mimic antibody (TCRm), or any combination thereof.

In some embodiments, type III NKT cells are modified to express a chimeric antigen receptor (CAR). In some embodiments, a CAR can be engineered using an antigen binding domain such that when the CAR is expressed on a cell (e.g., a type III NKT cell), the CAR and/or cell binds to the target antigen (e.g., CD19 or another exemplary antigen described herein). In some embodiments, the CAR sequences are cloned into a cell or cell population (e.g., a type III NKT cell or type III NKT cell population) and expanded using currently available protocols. In some embodiments, the cell or cell population comprises one or more polynucleotides encoding the CAR. In some embodiments, the cell or cell population is from a donor or a patient (e.g., a patient having or suspected of having a cancer, e.g., AML or another exemplary cancer described herein). In some embodiments, when used as a therapeutic agent, and when the cell or cell population is from a patient, the CAR-modified cell or cell population may be administered to the same patient and/or to another patient in need of such treatment. In some embodiments, when used as a therapeutic agent, and when the cell or cell population is from a donor, the CAR-modified cell or cell population may be administered to any patient in need of such treatment.

As used herein, the term “CAR-expressing” and “CAR-modified” when used to describe a cell or cell population refers to a cell or cell population that has been artificially engineered to comprise one or more polynucleotides encoding the sequence of a CAR peptide and which can transcribe, translate, and express the CAR peptide on the cell surface. In some embodiments, the CAR-expressing cell or cell population comprises a type III NKT cell or cell population. In some embodiments, the CAR-expressing cell or cell population comprises a CD3⁺CD56⁺ type III NKT cell or cell population. In some embodiments, the CAR-expressing cell or cell population comprises a CD3⁺CD4⁺CD56⁺ cell or cell population. In some embodiments, the CAR-expressing cell or cell population comprises a CD3⁺CD8⁺CD56⁺ cell or cell population. In some embodiments, the CAR-expressing cell or cell population comprises a CD3⁺CD4⁻CD8⁻CD56⁺ cell or cell population. In some embodiments, the CAR-expressing cell or cell population comprises a mixture of cells, e.g., a mixture of CD3⁺CD4⁺CD56⁺ and CD3⁺CD8⁺CD56⁺ cells or a mixture of CD3⁺CD4⁺CD56⁺, CD3⁺CD8⁺CD56⁺ and CD3⁺CD4⁻CD8⁻CD56⁺ cells. In some embodiments, when used as a therapeutic agent, the CAR-expressing cell or cell population administered to a subject may comprise a CAR-modified NKT cell, or a population of CAR-modified NKT cells, from the subject. In some embodiments, when used as a therapeutic agent, the CAR-expressing cell or cell population administered to a subject may comprise a CAR-modified NKT cell, or a population of CAR-modified NKT cells, from a donor.

In some embodiments, a CAR-modified cell or cell population can engage with and kill cells (e.g., malignant cancer cells) that express the target antigen (e.g., CD19). Methods and compositions for making and administering the disclosed CAR-based immunotherapies are provided herein. Exemplary methods for making CAR-based immunotherapies are also disclosed in, e.g., U.S. Publication Nos. 2015/0344844 and 2017/0218337, which are both incorporated herein by reference for such methods.

The terms “chimeric antigen receptor” and “CAR,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a CAR comprises at least an extracellular antigen binding domain, a transmembrane domain, and an intracellular signaling domain. In some embodiments, a CAR comprises at least an extracellular antigen binding domain, a transmembrane domain, and an intracellular signaling domain comprising a functional signaling domain derived from a stimulatory molecule and/or a costimulatory molecule. These domains may reside in a single polypeptide or a set of polypeptides. In some embodiments, the stimulatory molecule is the zeta chain associated with the T cell receptor complex. In some embodiments, the costimulatory molecule is 4-1BB, CD28, CD27, CD134 (OX40), ICOS, DAP10, and/or DAP12.

In some embodiments, a CAR comprises a chimeric fusion protein comprising an extracellular antigen binding domain, a transmembrane domain, and an intracellular signaling domain comprising: (i) a functional signaling domain derived from a stimulatory molecule; (ii) a functional signaling domain derived from a stimulatory molecule and a functional signaling domain derived from a costimulatory molecule; or (iii) a functional signaling domain derived from a stimulatory molecule and at least two functional signaling domains derived from one or more costimulatory molecule(s). In some embodiments, a CAR comprises an optional leader sequence at the N-terminus of the CAR fusion protein. In some embodiments, a CAR further comprises a leader sequence at the N-terminus of the extracellular antigen binding domain, wherein the leader sequence is optionally cleaved from the antigen binding domain during cellular processing and localization of the CAR to the cellular membrane.

In some embodiments, an antigen binding domain of a CAR comprises an antibody or an antigen binding fragment thereof. In some embodiments, the antigen binding domain and/or antibody comprises a monoclonal antibody, a polyclonal antibody, a synthetic antibody, a human antibody, a humanized antibody, or a single domain antibody (also known as a nanobody). In some embodiments, the antigen binding domain and/or antigen binding fragment comprises a single chain variable fragment (scFv) or a Fab fragment. In some embodiments, the antigen binding domain and/or antigen binding fragment comprises an scFv.

As used herein, the term “antibody” refers to any functional immunoglobulin molecule that recognizes and binds, e.g., specifically binds, to a target, such as a protein, polypeptide, carbohydrate, polynucleotide, lipid, or combinations of the foregoing through at least one antigen recognition site within the variable region of the immunoglobulin molecule. The term “antibody” encompasses antibodies having sequences from any source species, such as mouse, rabbit, goat, llama, alpaca, non-human primate, and human. The term further encompasses human antibodies, chimeric antibodies, humanized antibodies, and any modified immunoglobulin molecule containing an antigen recognition site, so long as it demonstrates the desired binding and/or biological activity. In some embodiments, an antibody possesses the ability to bind, e.g., specifically bind, a target antigen expressed on a cancer cell (e.g., CD19). An antibody can be generated using any suitable technology, e.g., recombinant expression, hybridoma technology, ribosome display, phage display, gene shuffling libraries, semi-synthetic, or fully synthetic libraries, or any combination thereof. The term “antibody” includes full-length antibodies as well as antigen binding domains and antigen binding fragments thereof. In some embodiments, an antibody used in the CARs and/or other constructs described herein is a full-length or intact antibody. In some embodiments, an antibody used in the CARs and/or other constructs described herein is a monoclonal antibody, a polyclonal antibody, a synthetic antibody, a human antibody, a humanized antibody, or a single domain antibody. In some embodiments, an antibody used in the CARs and/or other constructs described herein is an antigen binding domain or an antigen binding fragment of an antibody.

A “full-length” or “intact” antibody typically comprises at least two heavy (H) chains and two light (L) chains inter-connected by disulfide bonds. The recognized classes of immunoglobulin genes encoding antibody chains include the kappa, lambda, alpha, gamma, delta, epsilon, and mu constant region genes, as well as the myriad immunoglobulin variable region genes. Light chains are classified as either kappa or lambda. In some embodiments, an antibody comprises a kappa light chain. In some embodiments, an antibody comprises a lambda light chain. The kappa or lambda light chain may be selected from any kappa or lambda light chain sequence from any species. Heavy chains are classified as gamma, mu, alpha, delta, or epsilon, which in turn define the immunoglobulin classes, IgG, IgM, IgA, IgD and IgE, respectively. The four subclasses of IgG (IgG1, IgG2, IgG3, and IgG4) differ in their constant region and exhibit different effector functions.

Antibodies that may be used in the CARs and/or other constructs described herein also include antigen binding fragments. The term “antigen binding fragment” or “antigen binding portion” of an antibody, as used herein, refers to one or more fragments of a full-length antibody that retain the ability to bind, e.g., specifically bind, to the target antigen (e.g., CD19) and/or provide a function of the full-length antibody (e.g., the ability to specifically bind to CD19). Antigen binding functions of an antibody can be performed by fragments of a full-length antibody. Fragments can also be present in larger macromolecules, e.g., bispecific antibodies. Examples of such antibody fragments include a Fab fragment, a monovalent fragment comprising at least a VL, CL, VH, and CH1 domain; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment, which consists of a VH domain or a VL domain; an isolated complementarity determining region (CDR); and a half body, which comprises only one heavy chain and one light chain rather than the typical pairing of two heavy and two light chains on separate arms. Furthermore, although the two domains of the Fv fragment, VL and VH, are coded for by separate genes, they can be joined using recombinant methods, e.g., by an artificial peptide linker that enables them to be made as a single protein chain in which the VL and VH regions pair to form monovalent molecules (known as a single chain variable fragment (scFv)) (see, e.g., Bird et al., Science 1988; 242(4877):423-6; Huston et al., PNAS 1988; 85(16):5879-83). Such single chain antibodies include one or more antigen binding fragments or portions of an antibody. Other examples of antigen binding fragments include bispecific T cell engagers (BiTEs), which consist of two scFvs of different antibodies, or amino acid sequences from four different genes, on a single peptide chain. In some embodiments, an antigen binding fragment is a Fab fragment or an scFv. In some embodiments, an antigen binding fragment is an scFv.

In some embodiments, an antigen binding domain of a CAR comprises a cell-binding agent. In some embodiments, an antigen binding domain and/or cell-binding agent of a CAR comprises a DARPin, duobody, bicyclic peptide, nanobody, centyrin, MSH (melanocyte-stimulating hormone), receptor-Fc fusion molecule, T cell receptor structure, natural ligand (e.g., a receptor expressed in mature non-malignant and/or malignant B cells, including plasma cells, e.g., exemplary ligands to B-cell maturation antigen (BCMA) include, without limitation, B-cell activating factor (BAFF) and proliferation inducing ligand (APRIL)), steroid hormone (e.g., an androgen or estrogen), growth factor, colony-stimulating factor (e.g., EGF), or other non-antibody scaffold. In some embodiments, non-antibody scaffolds can broadly fall into two structural classes, namely domain-sized compounds (approximately 6-20 kDa) and constrained peptides (approximately 2-4 kDa). Exemplary domain-sized scaffolds include but are not limited to affibodies, affilins, anticalins, atrimers, DARPins, FN3 scaffolds (e.g., adnectins and centyrins), fynomers, Kunitz domains, pronectins, O-bodies, and receptor-Fc fusion proteins, whereas exemplary constrained peptides include avimers, bicyclic peptides, and Cys-knots. In some embodiments, an antigen binding domain and/or cell-binding agent of a CAR comprises an affibody, an affilin, an anticalin, an atrimer, a DARPin, a FN3 scaffold such as an adnectin or a centyrin, a fynomer, a Kunitz domain, a pronectin, an O-body, a receptor-Fc fusion protein, an avimer, a bicyclic peptide, and/or a Cys-knot. Non-antibody scaffolds are reviewed, e.g., in Vazquez-Lombardi et al., Drug Dis Today 2015; 20(10):1271-83.

In some embodiments, an antigen binding domain of a CAR is capable of binding to CD19, IGF1R, ROR1, BCMA, CD123, CD33, CD38, CD138, CLL-1, LILRB4, GD2, CD20, CD22, CD30, MSLN, EGFRvIII, EGFR, HER2, MUC1, EPCAM, PSMA, SLAMF7, GPC3, or PD-L1. In some embodiments, the antigen binding domain is capable of binding to CD19, IGF1R, or ROR1.

The term “CD19,” as used herein, refers to any native form of human B-lymphocyte antigen CD19 (CD19). The term encompasses full-length CD19 (e.g., UniProt Reference Sequence: P15391; SEQ ID NO: 1), as well as any form of human CD19 that may result from cellular processing. The term also encompasses functional variants or fragments of human CD19, including but not limited to splice variants, allelic variants, and isoforms that retain one or more biologic functions of human CD19 (i.e., variants and fragments are encompassed unless the context indicates that the term is used to refer to the wild-type protein only). CD19 can be isolated from a human, or may be produced recombinantly or by synthetic methods.

The term “IGF1R,” as used herein, refers to any native form of human insulin-like growth factor 1 receptor (IGF1R). The term encompasses full-length IGF1R (e.g., UniProt Reference Sequence: P08069; SEQ ID NO: 2), as well as any form of human IGF1R that may result from cellular processing. The term also encompasses functional variants or fragments of human IGF1R, including but not limited to splice variants, allelic variants, and isoforms that retain one or more biologic functions of human IGF1R (i.e., variants and fragments are encompassed unless the context indicates that the term is used to refer to the wild-type protein only). IGF1R can be isolated from a human, or may be produced recombinantly or by synthetic methods.

The term “ROR1,” as used herein, refers to any native form of human inactive tyrosine-protein kinase transmembrane receptor ROR1 (ROR1). The term encompasses full-length ROR1 (e.g., UniProt Reference Sequence: Q01973; SEQ ID NO: 3), as well as any form of human ROR1 that may result from cellular processing. The term also encompasses functional variants or fragments of human ROR1, including but not limited to splice variants, allelic variants, and isoforms that retain one or more biologic functions of human ROR1 (i.e., variants and fragments are encompassed unless the context indicates that the term is used to refer to the wild-type protein only). ROR1 can be isolated from a human, or may be produced recombinantly or by synthetic methods.

The term “BCMA,” as used herein, refers to any native form of human B-cell maturation antigen (BCMA). The term encompasses full-length BCMA (e.g., UniProt Reference Sequence: Q02223; SEQ ID NO: 4), as well as any form of human BCMA that may result from cellular processing. The term also encompasses functional variants or fragments of human BCMA, including but not limited to splice variants, allelic variants, and isoforms that retain one or more biologic functions of human BCMA (i.e., variants and fragments are encompassed unless the context indicates that the term is used to refer to the wild-type protein only). BCMA can be isolated from a human, or may be produced recombinantly or by synthetic methods.

The term “CD123,” as used herein, refers to any native form of human interleukin-3 receptor subunit alpha (CD123). The term encompasses full-length CD123 (e.g., UniProt Reference Sequence: P26951; SEQ ID NO: 5), as well as any form of human CD123 that may result from cellular processing. The term also encompasses functional variants or fragments of human CD123, including but not limited to splice variants, allelic variants, and isoforms that retain one or more biologic functions of human CD123 (i.e., variants and fragments are encompassed unless the context indicates that the term is used to refer to the wild-type protein only). CD123 can be isolated from a human, or may be produced recombinantly or by synthetic methods.

The term “CD33,” as used herein, refers to any native form of human myeloid cell surface antigen CD33 (CD33). The term encompasses full-length CD33 (e.g., UniProt Reference Sequence: P20138; SEQ ID NO: 6), as well as any form of human CD33 that may result from cellular processing. The term also encompasses functional variants or fragments of human CD33, including but not limited to splice variants, allelic variants, and isoforms that retain one or more biologic functions of human CD33 (i.e., variants and fragments are encompassed unless the context indicates that the term is used to refer to the wild-type protein only). CD33 can be isolated from a human, or may be produced recombinantly or by synthetic methods.

The term “CD38,” as used herein, refers to any native form of human ADP-ribosyl cyclase/cyclic ADP-ribose hydrolase 1 (CD38). The term encompasses full-length CD38 (e.g., UniProt Reference Sequence: P28907; SEQ ID NO: 7), as well as any form of human CD38 that may result from cellular processing. The term also encompasses functional variants or fragments of human CD38, including but not limited to splice variants, allelic variants, and isoforms that retain one or more biologic functions of human CD38 (i.e., variants and fragments are encompassed unless the context indicates that the term is used to refer to the wild-type protein only). CD38 can be isolated from a human, or may be produced recombinantly or by synthetic methods.

The term “CD138,” as used herein, refers to any native form of human syndecan-1 (CD138). The term encompasses full-length CD138 (e.g., UniProt Reference Sequence: P18827; SEQ ID NO: 8), as well as any form of human CD138 that may result from cellular processing. The term also encompasses functional variants or fragments of human CD138, including but not limited to splice variants, allelic variants, and isoforms that retain one or more biologic functions of human CD138 (i.e., variants and fragments are encompassed unless the context indicates that the term is used to refer to the wild-type protein only). CD138 can be isolated from a human, or may be produced recombinantly or by synthetic methods.

The term “CLL-1,” as used herein, refers to any native form of human C-type lectin-like molecule 1 (CLL-1). The term encompasses full-length CLL-1 (e.g., UniProt Reference Sequence: Q5QGZ9; SEQ ID NO: 9), as well as any form of human CLL-1 that may result from cellular processing. The term also encompasses functional variants or fragments of human CLL-1, including but not limited to splice variants, allelic variants, and isoforms that retain one or more biologic functions of human CLL-1 (i.e., variants and fragments are encompassed unless the context indicates that the term is used to refer to the wild-type protein only). CLL-1 can be isolated from a human, or may be produced recombinantly or by synthetic methods.

The term “LILRB4,” as used herein, refers to any native form of human leukocyte immunoglobulin-like receptor subfamily B member 4 (LILRB4). The term encompasses full-length LILRB4 (e.g., UniProt Reference Sequence: Q8NHJ6; SEQ ID NO: 10), as well as any form of human LILRB4 that may result from cellular processing. The term also encompasses functional variants or fragments of human LILRB4, including but not limited to splice variants, allelic variants, and isoforms that retain one or more biologic functions of human LILRB4 (i.e., variants and fragments are encompassed unless the context indicates that the term is used to refer to the wild-type protein only). LILRB4 can be isolated from a human, or may be produced recombinantly or by synthetic methods.

The term “GD2,” as used herein, refers to any native form of human ganglioside G2 (GD2). The term encompasses full-length GD2 (e.g., (2R,4R,5S,6S)-2-[3-[(2S,3S,4R,6S)-6-[(2S,3R,4R,5S,6R)-5-[(2S,3R,4R,5R,6R)-3-acetamido-4,5-dihydroxy-6-(hydroxymethyl)oxan-2-yl]oxy-2-[(2R,3S,4R,5R,6R)-4,5-dihydroxy-2-(hydroxymethyl)-6-[(E)-3-hydroxy-2-(octadecanoylamino)octadec-4-enoxy]oxan-3-yl]oxy-3-hydroxy-6-(hydroxymethyl)oxan-4-yl]oxy-3-amino-6-carboxy-4-hydroxyoxan-2-yl]-2,3-dihydroxypropoxy]-5-amino-4-hydroxy-6-(1,2,3-trihydroxypropyl)oxane-2-carboxylic acid), as well as any form of human GD2 that may result from cellular processing. The term also encompasses functional variants, fragments, or analogues of human GD2 that retain one or more biologic functions of human GD2 (i.e., variants, fragments, and analogues are encompassed unless the context indicates that the term is used to refer to the wild-type ganglioside only). GD2 can be isolated from a human, or may be produced by synthetic methods.

The term “CD20,” as used herein, refers to any native form of human B-lymphocyte antigen CD20 (CD20). The term encompasses full-length CD20 (e.g., UniProt Reference Sequence: P11836; SEQ ID NO: 11), as well as any form of human CD20 that may result from cellular processing. The term also encompasses functional variants or fragments of human CD20, including but not limited to splice variants, allelic variants, and isoforms that retain one or more biologic functions of human CD20 (i.e., variants and fragments are encompassed unless the context indicates that the term is used to refer to the wild-type protein only). CD20 can be isolated from a human, or may be produced recombinantly or by synthetic methods.

The term “CD22,” as used herein, refers to any native form of human B-cell receptor CD22 (CD22). The term encompasses full-length CD22 (e.g., UniProt Reference Sequence: P20273; SEQ ID NO: 12), as well as any form of human CD22 that may result from cellular processing. The term also encompasses functional variants or fragments of human CD22, including but not limited to splice variants, allelic variants, and isoforms that retain one or more biologic functions of human CD22 (i.e., variants and fragments are encompassed unless the context indicates that the term is used to refer to the wild-type protein only). CD22 can be isolated from a human, or may be produced recombinantly or by synthetic methods.

The term “CD30,” as used herein, refers to any native form of human tumor necrosis factor receptor superfamily member 8 (CD30). The term encompasses full-length CD30 (e.g., UniProt Reference Sequence: P28908; SEQ ID NO: 13), as well as any form of human CD30 that may result from cellular processing. The term also encompasses functional variants or fragments of human CD30, including but not limited to splice variants, allelic variants, and isoforms that retain one or more biologic functions of human CD30 (i.e., variants and fragments are encompassed unless the context indicates that the term is used to refer to the wild-type protein only). CD30 can be isolated from a human, or may be produced recombinantly or by synthetic methods.

The term “MSLN,” as used herein, refers to any native form of human mesothelin (MSLN). The term encompasses full-length MSLN (e.g., UniProt Reference Sequence: Q13421; SEQ ID NO: 14), as well as any form of human MSLN that may result from cellular processing. The term also encompasses functional variants or fragments of human MSLN, including but not limited to splice variants, allelic variants, and isoforms that retain one or more biologic functions of human MSLN (i.e., variants and fragments are encompassed unless the context indicates that the term is used to refer to the wild-type protein only). MSLN can be isolated from a human, or may be produced recombinantly or by synthetic methods.

The term “EGFRvIII,” as used herein, refers to any native form of human epidermal growth factor receptor variant III (EGFRvIII). The term encompasses full-length EGFRvIII (e.g., UniProt Reference Sequence: P00533-3; SEQ ID NO: 15), as well as any form of human EGFRvIII that may result from cellular processing. The term also encompasses functional variants or fragments of human EGFRvIII, including but not limited to splice variants, allelic variants, and isoforms that retain one or more biologic functions of human EGFRvIII (i.e., variants and fragments are encompassed unless the context indicates that the term is used to refer to the wild-type protein only). EGFRvIII can be isolated from a human, or may be produced recombinantly or by synthetic methods.

The term “EGFR,” as used herein, refers to any native form of human epidermal growth factor receptor (EGFR). The term encompasses full-length EGFR (e.g., UniProt Reference Sequence: P00533; SEQ ID NO: 16), as well as any form of human EGFR that may result from cellular processing. The term also encompasses functional variants or fragments of human EGFR, including but not limited to splice variants, allelic variants, and isoforms that retain one or more biologic functions of human EGFR (i.e., variants and fragments are encompassed unless the context indicates that the term is used to refer to the wild-type protein only). EGFR can be isolated from a human, or may be produced recombinantly or by synthetic methods.

The term “HER2,” as used herein, refers to any native form of human epidermal growth factor receptor 2 (HER2). The term encompasses full-length HER2 (e.g., UniProt Reference Sequence: P04626; SEQ ID NO: 17), as well as any form of human HER2 that may result from cellular processing. The term also encompasses functional variants or fragments of human HER2, including but not limited to splice variants, allelic variants, and isoforms that retain one or more biologic functions of human HER2 (i.e., variants and fragments are encompassed unless the context indicates that the term is used to refer to the wild-type protein only). HER2 can be isolated from a human, or may be produced recombinantly or by synthetic methods.

The term “MUC1,” as used herein, refers to any native form of human mucin-1 (MUC1). The term encompasses full-length MUC1 (e.g., UniProt Reference Sequence: P15941; SEQ ID NO: 18), as well as any form of human MUC1 that may result from cellular processing. The term also encompasses functional variants or fragments of human MUC1, including but not limited to splice variants, allelic variants, and isoforms that retain one or more biologic functions of human MUC1 (i.e., variants and fragments are encompassed unless the context indicates that the term is used to refer to the wild-type protein only). MUC1 can be isolated from a human, or may be produced recombinantly or by synthetic methods.

The term “EPCAM,” as used herein, refers to any native form of human epithelial cell adhesion molecule (EPCAM). The term encompasses full-length EPCAM (e.g., UniProt Reference Sequence: P16422; SEQ ID NO: 19), as well as any form of human EPCAM that may result from cellular processing. The term also encompasses functional variants or fragments of human EPCAM, including but not limited to splice variants, allelic variants, and isoforms that retain one or more biologic functions of human EPCAM (i.e., variants and fragments are encompassed unless the context indicates that the term is used to refer to the wild-type protein only). EPCAM can be isolated from a human, or may be produced recombinantly or by synthetic methods.

The term “PSMA,” as used herein, refers to any native form of human proteasome subunit alpha type-1 (PSMA). The term encompasses full-length PSMA (e.g., UniProt Reference Sequence: P25786; SEQ ID NO: 20), as well as any form of human PSMA that may result from cellular processing. The term also encompasses functional variants or fragments of human PSMA, including but not limited to splice variants, allelic variants, and isoforms that retain one or more biologic functions of human PSMA (i.e., variants and fragments are encompassed unless the context indicates that the term is used to refer to the wild-type protein only). PSMA can be isolated from a human, or may be produced recombinantly or by synthetic methods.

The term “SLAMF7,” as used herein, refers to any native form of human SLAM family member 7 (SLAMF7). The term encompasses full-length SLAMF7 (e.g., UniProt Reference Sequence: Q9NQ25; SEQ ID NO: 21), as well as any form of human SLAMF7 that may result from cellular processing. The term also encompasses functional variants or fragments of human SLAMF7, including but not limited to splice variants, allelic variants, and isoforms that retain one or more biologic functions of human SLAMF7 (i.e., variants and fragments are encompassed unless the context indicates that the term is used to refer to the wild-type protein only). SLAMF7 can be isolated from a human, or may be produced recombinantly or by synthetic methods.

The term “GPC3,” as used herein, refers to any native form of human glypican-3 (GPC3). The term encompasses full-length GPC3 (e.g., UniProt Reference Sequence: P51654; SEQ ID NO: 22), as well as any form of human GPC3 that may result from cellular processing. The term also encompasses functional variants or fragments of human GPC3, including but not limited to splice variants, allelic variants, and isoforms that retain one or more biologic functions of human GPC3 (i.e., variants and fragments are encompassed unless the context indicates that the term is used to refer to the wild-type protein only). GPC3 can be isolated from a human, or may be produced recombinantly or by synthetic methods.

The term “PD-L1,” as used herein, refers to any native form of human programmed cell death 1 ligand 1 (PD-L1). The term encompasses full-length PD-L1 (e.g., UniProt Reference Sequence: Q9NZQ7; SEQ ID NO: 23), as well as any form of human PD-L1 that may result from cellular processing. The term also encompasses functional variants or fragments of human PD-L1, including but not limited to splice variants, allelic variants, and isoforms that retain one or more biologic functions of human PD-L1 (i.e., variants and fragments are encompassed unless the context indicates that the term is used to refer to the wild-type protein only). PD-L1 can be isolated from a human, or may be produced recombinantly or by synthetic methods.

In some embodiments, an intracellular signaling domain of a CAR comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1BB, CD28, CD27, CD134 (OX40), ICOS, DAP10, or DAP12.

In some embodiments, type III NKT cells are modified to express a T cell receptor (TCR). In some embodiments, a TCR can be engineered using an antigen binding alpha chain and/or beta chain such that when the TCR is expressed on a cell (e.g., a type III NKT cell), the TCR and/or cell binds to the target antigen (e.g., WT1 or another exemplary antigen described herein). In some embodiments, the TCR sequences are cloned into a cell or cell population (e.g., a type III NKT cell or type III NKT cell population) and expanded using currently available protocols. In some embodiments, the cell or cell population comprises one or more polynucleotides encoding the TCR. In some embodiments, the cell or cell population is from a donor or a patient (e.g., a patient having or suspected of having a cancer, e.g., AML or another exemplary cancer described herein). In some embodiments, when used as a therapeutic agent, and when the cell or cell population is from a patient, the TCR-modified cell or cell population may be administered to the same patient and/or to another patient in need of such treatment. In some embodiments, when used as a therapeutic agent, and when the cell or cell population is from a donor, the TCR-modified cell or cell population may be administered to any patient in need of such treatment.

As used herein, the term “TCR-expressing” and “TCR-modified” when used to describe a cell or cell population refers to a cell or cell population that has been artificially engineered to comprise one or more polynucleotides encoding the sequence of a TCR peptide and which can transcribe, translate, and express the TCR peptide on the cell surface. In some embodiments, the TCR-expressing cell or cell population comprises a type III NKT cell or cell population. In some embodiments, the TCR-expressing cell or cell population comprises a CD3⁺CD56⁺ type III NKT cell or cell population. In some embodiments, the TCR-expressing cell or cell population comprises a CD3⁺CD4⁺CD56⁺ cell or cell population. In some embodiments, the TCR-expressing cell or cell population comprises a CD3⁺CD8⁺CD56⁺ cell or cell population. In some embodiments, the TCR-expressing cell or cell population comprises a CD3⁺CD4⁻CD8⁻CD56⁺ cell or cell population. In some embodiments, the TCR-expressing cell or cell population comprises a mixture of cells, e.g., a mixture of CD3⁺CD4⁺CD56⁺, CD3⁺CD8⁺CD56⁺ and CD3⁺CD4⁻CD8⁻CD56⁺ cells. In some embodiments, when used as a therapeutic agent, the TCR-expressing cell or cell population administered to a subject may comprise a TCR-modified NKT cell, or a population of TCR-modified NKT cells, from the subject. In some embodiments, when used as a therapeutic agent, the TCR-expressing cell or cell population administered to a subject may comprise a TCR-modified NKT cell, or a population of TCR-modified NKT cells, from a donor.

In some embodiments, a TCR-modified cell or cell population can engage with and kill cells (e.g., malignant cancer cells) that express the target antigen (e.g., WT1). Methods and compositions for making and administering the disclosed TCR-based immunotherapies are provided herein. Exemplary methods for making TCR-based immunotherapies are also disclosed in, e.g., U.S. Pat. No. 9,115,372, which is incorporated herein by reference for such methods.

The terms “T cell receptor” and “TCR,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a TCR comprises at least an alpha chain and a beta chain. These chains may reside in a single polypeptide or a set of polypeptides. In some embodiments, the alpha chain and/or the beta chain is capable of binding to an antigen.

In some embodiments, the TCR comprises an alpha chain and a beta chain. In some embodiments, both the alpha chain and the beta chain comprise a constant region (c) and a variable region (v). In some embodiments, the variable region determines antigen specificity. In some embodiments, the variable region recognizes a target antigen, e.g., an antigen ligand comprising a short contiguous amino acid sequence of a protein that is presented on the target cell by a major histocompatibility complex (MHC) molecule (also known as a human leukocyte antigen (HLA) molecule). In some embodiments, accessory adhesion molecules expressed by T cells, such as CD4 for MHC class II and CD8 for MHC class I, are also involved. In some embodiments, signal transduction of a TCR is through an associated invariant CD3 complex. In some embodiments, the CD3 complex comprises different CD3 proteins that form two heterodimers (CD3δε and CD3γε) and one homodimer (CD3.

In some embodiments, an alpha chain and/or a beta chain of a TCR is capable of binding to an antigen. In some embodiments, the alpha chain and/or beta chain is capable of binding to NY-ESO-1, WT1, or MAGE-A3.

The term “NY-ESO-1,” as used herein, refers to any native form of human cancer/testis antigen NY-ESO-1 (NY-ESO-1). The term encompasses full-length NY-ESO-1 (e.g., UniProt Reference Sequence: P78358; SEQ ID NO: 24), as well as any form of human NY-ESO-1 that may result from cellular processing. The term also encompasses functional variants or fragments of human NY-ESO-1, including but not limited to splice variants, allelic variants, and isoforms that retain one or more biologic functions of human NY-ESO-1 (i.e., variants and fragments are encompassed unless the context indicates that the term is used to refer to the wild-type protein only). NY-ESO-1 can be isolated from a human, or may be produced recombinantly or by synthetic methods.

The term “WT1,” as used herein, refers to any native form of human Wilms tumor protein (WT1). The term encompasses full-length WT1 (e.g., UniProt Reference Sequence: P19544; SEQ ID NO: 25), as well as any form of human WT1 that may result from cellular processing. The term also encompasses functional variants or fragments of human WT1, including but not limited to splice variants, allelic variants, and isoforms that retain one or more biologic functions of human WT1 (i.e., variants and fragments are encompassed unless the context indicates that the term is used to refer to the wild-type protein only). WT1 can be isolated from a human, or may be produced recombinantly or by synthetic methods.

The term “MAGE-A3,” as used herein, refers to any native form of human melanoma-associated antigen 3 (MAGE-A3). The term encompasses full-length MAGE-A3 (e.g., UniProt Reference Sequence: P43357; SEQ ID NO: 26), as well as any form of human MAGE-A3 that may result from cellular processing. The term also encompasses functional variants or fragments of human MAGE-A3, including but not limited to splice variants, allelic variants, and isoforms that retain one or more biologic functions of human MAGE-A3 (i.e., variants and fragments are encompassed unless the context indicates that the term is used to refer to the wild-type protein only). MAGE-A3 can be isolated from a human, or may be produced recombinantly or by synthetic methods.

In some embodiments, type III NKT cells are modified to express a T cell receptor mimic antibody (TCRm). In some embodiments, a TCRm can be engineered using an antigen binding domain such that when the TCRm is expressed on a cell (e.g., a type III NKT cell), the TCRm and/or cell binds to the target antigen (e.g., a composite antigen, e.g., a composite antigen comprising a peptide and a human leukocyte antigen (HLA) molecule, as described herein). In some embodiments, the TCRm sequences are cloned into a cell or cell population (e.g., a type III NKT cell or type III NKT cell population) and expanded using currently available protocols. In some embodiments, the cell or cell population comprises one or more polynucleotides encoding the TCRm. In some embodiments, the cell or cell population is from a donor or a patient (e.g., a patient having or suspected of having a cancer, e.g., AML or another exemplary cancer described herein). In some embodiments, when used as a therapeutic agent, and when the cell or cell population is from a patient, the TCRm-modified cell or cell population may be administered to the same patient and/or to another patient in need of such treatment. In some embodiments, when used as a therapeutic agent, and when the cell or cell population is from a donor, the TCRm-modified cell or cell population may be administered to any patient in need of such treatment.

As used herein, the term “TCRm-expressing” and “TCRm-modified” when used to describe a cell or cell population refers to a cell or cell population that has been artificially engineered to comprise one or more polynucleotides encoding the sequence of a TCRm peptide and which can transcribe, translate, and express the TCRm peptide on the cell surface. In some embodiments, the TCRm-expressing cell or cell population comprises a type III NKT cell or cell population. In some embodiments, the TCRm-expressing cell or cell population comprises a CD3⁺CD56⁺ type III NKT cell or cell population. In some embodiments, the TCRm-expressing cell or cell population comprises a CD3⁺CD4⁺CD56⁺ cell or cell population. In some embodiments, the TCRm-expressing cell or cell population comprises a CD3⁺CD8⁺CD56⁺ cell or cell population. In some embodiments, the TCRm-expressing cell or cell population comprises a CD3⁺CD4⁻CD8⁻CD56⁺ cell or cell population.

In some embodiments, the TCRm-expressing cell or cell population comprises a mixture of cells, e.g., a mixture of CD3⁺CD4⁺CD56⁺, CD3⁺CD8⁺CD56⁺ and CD3⁺CD4⁻CD8⁻CD56⁺ cells. In some embodiments, when used as a therapeutic agent, the TCRm-expressing cell or cell population administered to a subject may comprise a TCRm-modified NKT cell, or a population of TCRm-modified NKT cells, from the subject. In some embodiments, when used as a therapeutic agent, the TCRm-expressing cell or cell population administered to a subject may comprise a TCRm-modified NKT cell, or a population of TCRm-modified NKT cells, from a donor.

In some embodiments, a TCRm-modified cell or cell population can engage with and kill cells (e.g., malignant cancer cells) that express the target antigen (e.g., a composite antigen, e.g., a composite antigen comprising a peptide and a human leukocyte antigen (HLA) molecule, as described herein). Methods and compositions for making and administering the disclosed TCRm-based immunotherapies are provided herein. Exemplary methods for making TCRm-based immunotherapies are also disclosed in, e.g., U.S. Publication No. 2019/092876, which is incorporated herein by reference for such methods.

The terms “T cell receptor mimic antibody” and “TCRm,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a TCRm comprises at least an extracellular antigen binding domain, a transmembrane domain, and an intracellular signaling domain. In some embodiments, a TCRm comprises at least an extracellular antigen binding domain, a transmembrane domain, and an intracellular signaling domain comprising a functional signaling domain derived from a stimulatory molecule and/or a costimulatory molecule. These domains may reside in a single polypeptide or a set of polypeptides. In some embodiments, the stimulatory molecule is the zeta chain associated with the T cell receptor complex. In some embodiments, the costimulatory molecule is 4-1BB, CD28, CD27, CD134 (OX40), ICOS, DAP10, and/or DAP12.

In some embodiments, an antigen binding domain of a TCRm comprises an antibody or an antigen binding fragment thereof. In some embodiments, the antigen binding domain and/or antibody comprises a monoclonal antibody, a polyclonal antibody, a synthetic antibody, a human antibody, a humanized antibody, or a single domain antibody. In some embodiments, the antigen binding domain and/or antigen binding fragment comprises a single chain variable fragment (scFv) or a Fab fragment. In some embodiments, the antigen binding domain and/or antigen binding fragment comprises an scFv.

In some embodiments, an antigen binding domain of a TCRm comprises a cell-binding agent. In some embodiments, an antigen binding domain and/or cell-binding agent of a TCRm comprises a DARPin, duobody, bicyclic peptide, nanobody, centyrin, MSH (melanocyte-stimulating hormone), receptor-Fc fusion molecule, T cell receptor structure, natural ligand (e.g., a receptor expressed in mature non-malignant and/or malignant B cells, including plasma cells, e.g., exemplary ligands to B-cell maturation antigen (BCMA) include, without limitation, B-cell activating factor (BAFF) and proliferation inducing ligand (APRIL)), steroid hormone (e.g., an androgen or estrogen), growth factor, colony-stimulating factor (e.g., EGF), or other non-antibody scaffold. In some embodiments, an antigen binding domain and/or cell-binding agent of a TCRm comprises an affibody, an affilin, an anticalin, an atrimer, a DARPin, a FN3 scaffold such as an adnectin or a centyrin, a fynomer, a Kunitz domain, a pronectin, an O-body, a receptor-Fc fusion protein, an avimer, a bicyclic peptide, and/or a Cys-knot.

In some embodiments, an antigen binding domain of a TCRm is capable of binding to a composite antigen comprising a peptide and a human leukocyte antigen (HLA) molecule.

In some embodiments, the HLA molecule is a class I HLA molecule. In some embodiments, the HLA molecule is a class I HLA binding peptide. In some embodiments, a class I HLA binding peptide is about 9 or 10 amino acids in length. Exemplary class I HLA binding peptides include but are not limited to: NY-ESO-1-derived HLA-A*0201 binding peptide SLLMWITQC (SEQ ID NO: 29); NY-ESO-1-derived HLA-A*0201 binding peptide SLLMWITQV (SEQ ID NO: 30); WT1-derived HLA-A*0201 binding peptide RMFPNAPYL (SEQ ID NO: 31); MAGE-A3-derived HLA-A*0201 binding peptide KVAELVHFL (SEQ ID NO: 32); MAGE-A3-derived HLA-A*0201 binding peptide EVDPIGHLY (SEQ ID NO: 33); MAGE-A4-derived HLA-A*2402 binding peptide NYKRCFPVI (SEQ ID NO: 34); MART-1-derived HLA-A*0201 binding peptide AAGIGILTV (SEQ ID NO: 35); and HPV E7-derived HLA-A*0201 binding peptide YMLDLQPET (SEQ ID NO: 36).

In some embodiments, an antigen binding domain of a TCRm is capable of binding to a composite antigen comprising a HLA molecule having an amino acid sequence of SLLMWITQC (SEQ ID NO: 29); SLLMWITQV (SEQ ID NO: 30); RMFPNAPYL (SEQ ID NO: 31); KVAELVHFL (SEQ ID NO: 32); EVDPIGHLY (SEQ ID NO: 33); NYKRCFPVI (SEQ ID NO: 34); AAGIGILTV (SEQ ID NO: 35); and/or YMLDLQPET (SEQ ID NO: 36).

In some embodiments, the HLA molecule is a class II HLA molecule. In some embodiments, the HLA molecule is a class II HLA binding peptide. In some embodiments, a class II HLA binding peptide is about 13 to about 25 amino acids in length. Exemplary class II HLA binding peptides include but are not limited to: NY-ESO-1-derived HLA-DRB1*0401 binding peptide LKEFTVSGNILTIRL (SEQ ID NO: 37); NY-ESO-1-derived HLA-DRB1*0401 binding peptide LPVPGVLLKEFTVSGNILTI (SEQ ID NO: 38); MAGE-A3-derived HLA-DRB1*1101 binding peptide TSYVKVLHHMVKISG (SEQ ID NO: 39); and MART-1-derived HLA-DRB1*0401 binding peptide RNGYRALMDKSLHVGTQCALTRR (SEQ ID NO: 40).

In some embodiments, an antigen binding domain of a TCRm is capable of binding to a composite antigen comprising a HLA molecule having an amino acid sequence of LKEFTVSGNILTIRL (SEQ ID NO: 37); LPVPGVLLKEFTVSGNILTI (SEQ ID NO: 38); TSYVKVLHHMVKISG (SEQ ID NO: 39); and/or RNGYRALMDKSLHVGTQCALTRR (SEQ ID NO: 40).

In addition to a HLA molecule, in some embodiments, a composite antigen comprises a peptide. In some embodiments, the peptide comprises an AFP peptide. In some embodiments, the composite antigen comprises an AFP peptide and a HLA-A2 molecule.

The term “AFP” or “AFP peptide,” as used herein, refers to any native form of human alpha fetoprotein (AFP). The term encompasses full-length AFP (e.g., UniProt Reference Sequence: P02771; SEQ ID NO: 27), as well as any form of human AFP that may result from cellular processing. The term also encompasses functional variants or fragments of human AFP, including but not limited to splice variants, allelic variants, and isoforms that retain one or more biologic functions of human AFP (i.e., variants and fragments are encompassed unless the context indicates that the term is used to refer to the wild-type protein only). AFP can be isolated from a human, or may be produced recombinantly or by synthetic methods.

In some embodiments, the peptide comprises a preferentially expressed antigen in melanoma (PRAME) peptide. In some embodiments, the PRAME peptide comprises an amino acid sequence of ALYVDSLFFL (SEQ ID NO: 41). In some embodiments, the composite antigen comprises a PRAME peptide and a HLA-A*0201 molecule.

The term “PRAME” or “PRAME peptide,” as used herein, refers to any native form of human preferentially expressed antigen in melanoma (PRAME). The term encompasses full-length PRAME (e.g., UniProt Reference Sequence: P78395; SEQ ID NO: 28), as well as any form of human PRAME that may result from cellular processing. The term also encompasses functional variants or fragments of human PRAME, including but not limited to splice variants, allelic variants, and isoforms that retain one or more biologic functions of human PRAME (i.e., variants and fragments are encompassed unless the context indicates that the term is used to refer to the wild-type protein only). PRAME can be isolated from a human, or may be produced recombinantly or by synthetic methods.

In some embodiments, the peptide comprises a WT1 peptide. In some embodiments, the composite antigen comprises a WT1 peptide and a HLA-A2 molecule.

The term “WT1” or “WT1 peptide,” as used herein, refers to any native form of human Wilms tumor protein (WT1). The term encompasses full-length WT1 (e.g., UniProt Reference Sequence: P19544; SEQ ID NO: 25), as well as any form of human WT1 that may result from cellular processing. The term also encompasses functional variants or fragments of human WT1, including but not limited to splice variants, allelic variants, and isoforms that retain one or more biologic functions of human WT1 (i.e., variants and fragments are encompassed unless the context indicates that the term is used to refer to the wild-type protein only). WT1 can be isolated from a human, or may be produced recombinantly or by synthetic methods.

In some embodiments, an intracellular signaling domain of a TCRm comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1 BB, CD28, CD27, CD134 (OX40), ICOS, DAP10, or DAP12.

In some embodiments, type III NKT cells of the present disclosure (e.g., CD3⁺CD56⁺ type III NKT cells) are further modified to comprise an exogenous cytokine, growth factor, antibody or antigen binding fragment, or any combination thereof. In some embodiments, the antibody or antigen binding fragment comprises a bispecific T cell engager (BiTE).

TABLE 1
Exemplary Target Antigen Amino Acid Sequences

| Antigen | SEQ ID NO | Amino Acid Sequence |
|---|---|---|
| CD19 | 1 | MPPPRLLFELLFLTPMEVRPEEPLVVKVEEGDNAVLQCLKGTS |
| | | DGPTQQLTWSRESPLKPFLKLSLGLPGLGIHMRPLAIWLFIFNVS |
| | | QQMGGFYLCQPGPPSEKAWQPGWTVNVEGSGELFRWNVSDLGGLG |
| | | CGLKNRSSEGPSSPSGKLMSPKLYVWAKDRPEIWEGEPPCLPPRD |
| | | SLNQSLSQDLTMAPGSTLWLSCGVPPDSVSRGPLSWTHVHPKGPK |
| | | SLLSLELKDDRPARDMWVMETGLLLPRATAQDAGKYYCHRGNLTM |
| | | SFHLEITARPVLWHWLLRTGGWKVSAVTLAYLIFCLCSLVGILHL |
| | | QRALVLRRKRKRMTDPTRRFFKVTPPPGSGPQNQYGNVLSLPTPT |
| | | SGLGRAQRWAAGLGGTAPSYGNPSSDVQADGALGSRSPPGVGPEE |
| | | EEGEGYEEPDSEEDSEFYENDSNLGQDQLSQDGSGYENPEDEPLG |
| | | PEDEDSFSNAESYENEDEELTQPVARTMDFLSPHGSAWDPSREAT |
| | | SLGSQSYEDMRGILYAAPQLRSIRGQPGPNHEEDADSYENMDNPD |
| | | GPDPAWGGGGRMGTWSTR |
| IGF1R | 2 | MKSGSGGGSPTSLWGLLFLSAALSLWPTSGEICGPGIDIRNDY |
| | | QQLKRLENCTVIEGYLHILLISKAEDYRSYRFPKLTVITEYLLLF |
| | | RVAGLESLGDLFPNLTVIRGWKLFYNYALVIFEMTNLKDIGLYNL |
| | | RNITRGAIRIEKNADLCYLSTVDWSLILDAVSNNYIVGNKPPKEC |
| | | GDLCPGTMEEKPMCEKTTINNEYNYRCWTTNRCQKMCPSTCGKRA |
| | | CTENNECCHPECLGSCSAPDNDTACVACRHYYYAGVCVPACPPNT |
| | | YRFEGWRCVDRDFCANILSAESSDSEGFVIHDGECMQECPSGEIR |
| | | NGSQSMYCIPCEGPCPKVCEEEKKTKTIDSVTSAQMLQGCTIFKG |
| | | NLLINTRRGNNIASELENFMGLIEVVTGYVKIRHSHALVSLSFLK |
| | | NLRLILGEEQLEGNYSFYVLDNQNLQQLWDWDHRNLTIKAGKMYF |
| | | AFNPKLCVSEIYRMEEVTGTKGRQSKGDINTRNNGERASCESDVL |
| | | HETSTTTSKNRIIITWHRYRPPDYRDLISFTVYYKEAPFKNVTEY |
| | | DGQDACGSNSWNMVDVDLPPNKDVEPGILLHGLKPWTQYAVYVKA |
| | | VTLTMVENDHIRGAKSEILYIRTNASVPSIPLDVLSASNSSSQLI |
| | | VKWNPPSLPNGNLSYYIVRWQRQPQDGYLYRHNYCSKDKIPIRKY |
| | | ADGTIDIEEVTENPKTEVCGGEKGPCCACPKTEAEKQAEKEEAEY |
| | | RKVFENFLHNSIFVPRPERKRRDVMQVANTTMSSRSRNTTAADTY |
| | | NITDPEELETEYPFFESRVDNKERTVISNLRPFTLYRIDIHSCNH |
| | | EAEKLGCSASNFVFARTMPAEGADDIPGPVTWEPRPENSIFLKWP |
| | | EPENPNGLILMYEIKYGSQVEDQRECVSRQEYRKYGGAKLNRLNP |
| | | GNYTARIQATSLSGNGSWTDPVFFYVQAKTGYENFIHLIIALPVA |
| | | VLLIVGGLVIMLYVFHRKRNNSRLGNGVLYASVNPEYFSAADVYV |
| | | PDEWEVAREKITMSRELGQGSFGMVYEGVAKGVVKDEPETRVAIK |
| | | TVNEAASMRERIEFLNEASVMKEENCHHVVRLLGVVSQGQPILVI |
| | | MELMTRGDLKSYLRSLRPEMENNPVLAPPSLSKMIQMAGEIADGM |
| | | AYLNANKFVHRDLAARNCMVAEDFTVKIGDFGMTRDIYETDYYRK |
| | | GGKGLLPVRWMSPESLKDGVFTTYSDVWSFGVVLWEIATLAEQPY |
| | | QGLSNEQVLRFVMEGGLLDKPDNCPDMLFELMRMCWQYNPKMRPS |
| | | FLEIISSIKEEMEPGFREVSFYYSEENKLPEPEELDLEPENMESV |
| | | PLDPSASSSSLPLPDRHSGHKAENGPGPGVLVLRASFDERQPYAH |
| | | MNGGRKNERALPLPQSSTC |
| ROR1 | 3 | MHRPRRRGTRPPLLALLAALLLAARGAAAQETELSVSAELVPT |
| | | SSWNISSELNKDSYLTLDEPMNNITTSLGQTAELHCKVSGNPPPT |
| | | IRWEKNDAPVVQEPRRLSERSTIYGSRLRIRNLDTTDTGYFQCVA |
| | | TNGKEVVSSTGVLFVKFGPPPTASPGYSDEYEEDGECQPYRGIAC |
| | | ARFIGNRTVYMESLHMQGEIENQITAAFTMIGTSSHLSDKCSQFA |
| | | IPSLCHYAFPYCDETSSVPKPRDLCRDECEILENVLCQTEYIFAR |
| | | SNPMILMRLKLPNCEDLPQPESPEAANCIRIGIPMADPINKNHKC |
| | | YNSTGVDYRGTVSVTKSGRQCQPWNSQYPHTHTFTALRFPELNGG |
| | | HSYCRNPGNQKEAPWCFILDENEKSDLCDIPACDSKDSKEKNKME |
| | | ILYILVPSVAIPLAIALLFFFICVCRNNQKSSSAPVQRQPKHVRG |
| | | QNVEMSMLNAYKPKSKAKELPLSAVRFMEELGECAFGKIYKGHLY |
| | | LPGMDHAQLVAIKTLKDYNNPQQWTEFQQEASLMAELHHPNIVCL |
| | | LGAVTQEQPVCMLFEYINQGDLHEFLIMRSPHSDVGCSSDEDGTV |
| | | KSSLDHGDFLHIAIQIAAGMEYLSSHFFVHKDLAARNILIGEQLH |
| | | VKISDLGLSREIYSADYYRVQSKSLLPIRWMPPEAIMYGKFSSDS |
| | | DIWSFGVVLWEIFSFGLQPYYGFSNQEVIEMVRKRQLLPCSEDCP |
| | | PRMYSLMTECWNEIPSRRPRFKDIHVRLRSWEGLSSHTSSTIPSG |
| | | GNATTQTTSLSASPVSNLSNPRYPNYMFPSQGITPQGQIAGFIGP |
| | | PIPQNQRFIPINGYPIPPGYAAFPAAHYQPTGPPRVIQHCPPPKS |
| | | RSPSSASGSTSTGHVTSLPSSGSNQEANIPLLPHMSIPNHPGGMG |
| | | ITVFGNKSQKPYKIDSKQASLLGDANIHGHTESMISAEL |
| BCMA | 4 | MLQMAGQCSQNEYFDSLLHACIPCQLRCSSNTPPLTCQRYCNA |
| | | SVTNSVKGTNAILWTCLGLSLIISLAVEVLMELLRKINSEPLKDE |
| | | FKNTGSGLLGMANIDLEKSRTGDEIILPRGLEYTVEECTCEDCIK |
| | | SKPKVDSDHCFPLPAMEEGATILVTTKTNDYCKSLPAALSATEIE |
| | | KSISAR |
| CD123 | 5 | MVLLWLTLLLIALPCLLQTKEDPNPPITNLRMKAKAQQLTWDL |
| | | NRNVTDIECVKDADYSMPAVNNSYCQFGAISLCEVTNYTVRVANP |
| | | PFSTWILFPENSGKPWAGAENLTCWIHDVDFLSCSWAVGPGAPAD |
| | | VQYDLYLNVANRRQQYECLHYKTDAQGTRIGCRFDDISRLSSGSQ |
| | | SSHILVRGRSAAFGIPCTDKEVVFSQIEILTPPNMTAKCNKTHSF |
| | | MHWKMRSHENRKFRYELQIQKRMQPVITEQVRDRTSFQLLNPGTY |
| | | TVQIRARERVYEFLSAWSTPQRFECDQEEGANTRAWRTSLLIALG |
| | | TLLALVCVFVICRRYLVMQRLFPRIPHMKDPIGDSFQNDKLVVWE |
| | | AGKAGLEECLVTEVQVVQKT |
| CD33 | 6 | MPLLLLLPLLWAGALAMDPNFWLQVQESVTVQEGLCVLVPCIF |
| | | FHPIPYYDKNSPVHGYWFREGAIISRDSPVATNKLDQEVQEETQG |
| | | RFRLLGDPSRNNCSLSIVDARRRDNGSYFFRMERGSTKYSYKSPQ |
| | | LSVHVTDLTHRPKILIPGTLEPGHSKNLTCSVSWACEQGTPPIFS |
| | | WLSAAPTSLGPRTTHSSVLIITPRPQDHGTNLTCQVKFAGAGVTT |
| | | ERTIQLNVTYVPQNPTTGIFPGDGSGKQETRAGVVHGAIGGAGVT |
| | | ALLALCLCLIFFIVKTHRRKAARTAVGRNDTHPTTGSASPKHQKK |
| | | SKLHGPTETSSCSGAAPTVEMDEELHYASLNFHGMNPSKDTSTEY |
| | | SEVRTQ |
| CD38 | 7 | MANCEFSPVSGDKPCCRLSRRAQLCLGVSILVLILVVVLAVVV |
| | | PRWRQQWSGPGTTKRFPETVLARCVKYTEIHPEMRHVDCQSVWDA |
| | | FKGAFISKHPCNITEEDYQPLMKLGTQTVPCNKILLWSRIKDLAH |
| | | QFTQVQRDMFTLEDTLLGYLADDLTWCGEFNTSKINYQSCPDWRK |
| | | DCSNNPVSVFWKTVSRRFAEAACDVVHVMLNGSRSKIFDKNSTFG |
| | | SVEVHNLQPEKVQTLEAWVIHGGREDSRDLCQDPTIKELESIISK |
| | | RNIQFSCKNIYRPDKFLQCVKNPEDSSCTSEI |
| CD138 | 8 | MRRAALWLWLCALALSLQPALPQIVATNLPPEDQDGSGDDSDN |
| | | FSGSGAGALQDITLSQQTPSTWKDTQLLTAIPTSPEPTGLEATAA |
| | | STSTLPAGEGPKEGEAVVLPEVEPGLTAREQEATPRPRETTQLPT |
| | | THLASTTTATTAQEPATSHPHRDMQPGHHETSTPAGPSQADLHTP |
| | | HTEDGGPSATERAAEDGASSQLPAAEGSGEQDFTFETSGENTAVV |
| | | AVEPDRRNQSPVDQGATGASQGLLDRKEVLGGVIAGGLVGLIFAV |
| | | CLVGFMLYRMKKKDEGSYSLEEPKQANGGAYQKPTKQEEFYA |
| CLL-1 | 9 | MSEEVTYADLQFQNSSEMEKIPEIGKFGEKAPPAPSHVWRPAA |
| | | LFLTLLCLLLLIGLGVLASMFHVTLKIEMKKMNKLQNISEELQRN |
| | | ISLQLMSNMNISNKIRNLSTTLQTIATKLCRELYSKEQEHKCKPC |
| | | PRRWIWHKDSCYFLSDDVQTWQESKMACAAQNASLLKINNKNALE |
| | | FIKSQSRSYDYWLGLSPEEDSTRGMRVDNIINSSAWVIRNAPDLN |
| | | NMYCGYINRLYVQYYHCTYKKRMICEKMANPVQLGSTYFREA |
| LILRB4 | 10 | MIPTFTALLCLGLSLGPRTHMQAGPLPKPTLWAEPGSVISWGN |
| | | SVTIWCQGTLEAREYRLDKEESPAPWDRQNPLEPKNKARFSIPSM |
| | | TEDYAGRYRCYYRSPVGWSQPSDPLELVMTGAYSKPTLSALPSPL |
| | | VTSGKSVTLLCQSRSPMDTFLLIKERAAHPLLHLRSEHGAQQHQA |
| | | EFPMSPVTSVHGGTYRCFSSHGFSHYLLSHPSDPLELIVSGSLED |
| | | PRPSPTRSVSTAAGPEDQPLMPTGSVPHSGLRRHWEVLIGVLVVS |
| | | ILLLSLLLFLLLQHWRQGKHRTLAQRQADFQRPPGAAEPEPKDGG |
| | | LQRRSSPAADVQGENFCAAVKNTQPEDGVEMDTRQSPHDEDPQAV |
| | | TYAKVKHSRPRREMASPPSPLSGEFLDTKDRQAEEDRQMDTEAAA |
| | | SEAPQDVTYAQLHSFTLRQKATEPPPSQEGASPAEPSVYATLAIH |
| CD20 | 11 | MTTPRNSVNGTFPAEPMKGPIAMQSGPKPLFRRMSSLVGPTQS |
| | | FFMRESKTLGAVQIMNGLFHIALGGLLMIPAGIYAPICVTVWYPL |
| | | WGGIMYIISGSLLAATEKNSRKCLVKGKMIMNSLSLFAAISGMIL |
| | | SIMDILNIKISHFLKMESLNFIRAHTPYINTYNCEPANPSEKNSP |
| | | STQYCYSIQSLFLGILSVMLIFAFFQELVIAGIVENEWKRTCSRP |
| | | KSNIVLLSAEEKKEQTIEIKEEVVGLTETSSQPKNEEDIEIIPIQ |
| | | EEEEEETETNFPEPPQDQESSPTENDSSP |
| CD22 | 12 | MHLLGPWLLLLVLEYLAFSDSSKWVFEHPETLYAWEGACVWIP |
| | | CTYRALDGDLESFILEHNPEYNKNTSKEDGTRLYESTKDGKVPSE |
| | | QKRVQFLGDKNKNCTLSIHPVHLNDSGQLGLRMESKTEKWMERIH |
| | | LNVSERPFPPHIQLPPEIQESQEVTLTCLLNFSCYGYPIQLQWLL |
| | | EGVPMRQAAVTSTSLTIKSVETRSELKESPQWSHHGKIVTCQLQD |
| | | ADGKFLSNDTVQLNVKHTPKLEIKVTPSDAIVREGDSVTMTCEVS |
| | | SSNPEYTTVSWLKDGTSLKKQNTFTLNLREVTKDQSGKYCCQVSN |
| | | DVGPGRSEEVFLQVQYAPEPSTVQILHSPAVEGSQVEFLCMSLAN |
| | | PLPTNYTWYHNGKEMQGRTEEKVHIPKILPWHAGTYSCVAENILG |
| | | TGQRGPGAELDVQYPPKKVTTVIQNPMPIREGDTVTLSCNYNSSN |
| | | PSVTRYEWKPHGAWEEPSLGVLKIQNVGWDNTTIACAACNSWCSW |
| | | ASPVALNVQYAPRDVRVRKIKPLSEIHSGNSVSLQCDFSSSHPKE |
| | | VQFFWEKNGRLLGKESQLNFDSISPEDAGSYSCWVNNSIGQTASK |
| | | AWTLEVLYAPRRLRVSMSPGDQVMEGKSATLTCESDANPPVSHYT |
| | | WFDWNNQSLPYHSQKLRLEPVKVQHSGAYWCQGTNSVGKGRSPLS |
| | | TLTVYYSPETIGRRVAVGLGSCLAILILAICGLKLQRRWKRTQSQ |
| | | QGLQENSSGQSFFVRNKKVRRAPLSEGPHSLGCYNPMMEDGISYT |
| | | TLRFPEMNIPRTGDAESSEMQRPPPDCDDTVTYSALHKRQVGDYE |
| | | NVIPDFPEDEGIHYSELIQFGVGERPQAQENVDYVILKH |
| CD30 | 13 | MRVLLAALGLLFLGALRAFPQDRPFEDTCHGNPSHYYDKAVRR |
| | | CCYRCPMGLFPTQQCPQRPTDCRKQCEPDYYLDEADRCTACVTCS |
| | | RDDLVEKTPCAWNSSRVCECRPGMFCSTSAVNSCARCFFHSVCPA |
| | | GMIVKFPGTAQKNTVCEPASPGVSPACASPENCKEPSSGTIPQAK |
| | | PTPVSPATSSASTMPVRGGTRLAQEAASKLTRAPDSPSSVGRPSS |
| | | DPGLSPTQPCPEGSGDCRKQCEPDYYLDEAGRCTACVSCSRDDLV |
| | | EKTPCAWNSSRTCECRPGMICATSATNSCARCVPYPICAAETVTK |
| | | PQDMAEKDTTFEAPPLGTQPDCNPTPENGEAPASTSPTQSLLVDS |
| | | QASKTLPIPTSAPVALSSTGKPVLDAGPVLFWVILVLVVVVGSSA |
| | | FLLCHRRACRKRIRQKLHLCYPVQTSQPKLELVDSRPRRSSTQLR |
| | | SGASVTEPVAEERGLMSQPLMETCHSVGAAYLESLPLQDASPAGG |
| | | PSSPRDLPEPRVSTEHTNNKIEKIYIMKADTVIVGTVKAELPEGR |
| | | GLAGPAEPELEEELEADHTPHYPEQETEPPLGSCSDVMLSVEEEG |
| | | KEDPLPTAASGK |
| MSLN | 14 | MALPTARPLLGSCGTPALGSLLFLLFSLGWVQPSRTLAGETGQ |
| | | EAAPLDGVLANPPNISSLSPRQLLGFPCAEVSGLSTERVRELAVA |
| | | LAQKNVKLSTEQLRCLAHRLSEPPEDLDALPLDLLLFLNPDAFSG |
| | | PQACTREFSRITKANVDLLPRGAPERQRLLPAALACWGVRGSLLS |
| | | EADVRALGGLACDLPGRFVAESAEVLLPRLVSCPGPLDQDQQEAA |
| | | RAALQGGGPPYGPPSTWSVSTMDALRGLLPVLGQPIIRSIPQGIV |
| | | AAWRQRSSRDPSWRQPERTILRPRFRREVEKTACPSGKKAREIDE |
| | | SLIFYKKWELEACVDAALLATQMDRVNAIPFTYEQLDVLKHKLDE |
| | | LYPQGYPESVIQHLGYLFLKMSPEDIRKWNVTSLETLKALLEVNK |
| | | GHEMSPQAPRRPLPQVATLIDRFVKGRGQLDKDTLDTLTAFYPGY |
| | | LCSLSPEELSSVPPSSIWAVRPQDLDTCDPRQLDVLYPKARLAFQ |
| | | NMNGSEYFVKIQSFLGGAPTEDLKALSQQNVSMDLATFMKLRTDA |
| | | VLPLTVAEVQKLLGPHVEGLKAEERHRPVRDWILRQRQDDLDTLG |
| | | LGLQGGIPNGYLVLDLSMQEALSGTPCLLGPGPVLTVLALLLAST |
| | | LA |
| EGFRvIII | 15 | MRPSGTAGAALLALLAALCPASRALEEKKVCQGTSNKLTQLGT |
| | | FEDHFLSLQRMFNNCEVVLGNLEITYVQRNYDLSFLKTIQEVAGY |
| | | VLIALNTVERIPLENLQIIRGNMYYENSYALAVLSNYDANKTGLK |
| | | ELPMRNLQEILHGAVRFSNNPALCNVESIQWRDIVSSDELSNMSM |
| | | DFQNHLGSCQKCDPSCPNGSCWGAGEENCQKLTKIICAQQCSGRC |
| | | RGKSPSDCCHNQCAAGCTGPRESDCLVCRKFRDEATCKDTCPPLM |
| | | LYNPTTYQMDVNPEGKYSFGATCVKKCPRNYVVTDHGSCVRACGA |
| | | DSYEMEEDGVRKCKKCEGPCRKVCNGIGIGEFKDSLSINATNIKH |
| | | FKNCTSISGDLHILPVAFRGDSFTHIPPLDPQELDILKTVKEITG |
| | | FLLIQAWPENRTDLHAFENLEIIRGRTKQHGQFSLAVVSLNITSL |
| | | GLRSLKEISDGDVIISGNKNLCYANTINWKKLFGTSGQKTKIISN |
| | | RGENSCKATGQVCHALCSPEGCWGPEPRDCVSCRNVSRGRECVDK |
| | | CNLLEGEPREFVENSECIQCHPECLPQAMNITCTGRGPDNCIQCA |
| | | HYIDGPHCVKTCPAGVMGENNTLVWKYADAGHVCHLCHPNCTYGP |
| | | GNESLKAMLFCLFKLSSCNQSNDGSVSHQSGSPAAQESCLGWIPS |
| | | LLPSEFQLGWGGCSHLHAWPSASVIITASSCH |
| EGFR | 16 | MRPSGTAGAALLALLAALCPASRALEEKKVCQGTSNKLTQLGT |
| | | FEDHFLSLQRMFNNCEVVLGNLEITYVQRNYDLSFLKTIQEVAGY |
| | | VLIALNTVERIPLENLQIIRGNMYYENSYALAVLSNYDANKTGLK |
| | | ELPMRNLQEILHGAVRFSNNPALCNVESIQWRDIVSSDELSNMSM |
| | | DFQNHLGSCQKCDPSCPNGSCWGAGEENCQKLTKIICAQQCSGRC |
| | | RGKSPSDCCHNQCAAGCTGPRESDCLVCRKFRDEATCKDTCPPLM |
| | | LYNPTTYQMDVNPEGKYSFGATCVKKCPRNYVVTDHGSCVRACGA |
| | | DSYEMEEDGVRKCKKCEGPCRKVCNGIGIGEFKDSLSINATNIKH |
| | | FKNCTSISGDLHILPVAFRGDSFTHIPPLDPQELDILKTVKEITG |
| | | FLLIQAWPENRTDLHAFENLEIIRGRTKQHGQFSLAVVSLNITSL |
| | | GLRSLKEISDGDVIISGNKNLCYANTINWKKLFGTSGQKTKIISN |
| | | RGENSCKATGQVCHALCSPEGCWGPEPRDCVSCRNVSRGRECVDK |
| | | CNLLEGEPREFVENSECIQCHPECLPQAMNITCTGRGPDNCIQCA |
| | | HYIDGPHCVKTCPAGVMGENNTLVWKYADAGHVCHLCHPNCTYGC |
| | | TGPGLEGCPTNGPKIPSIATGMVGALLLLLVVALGIGLFMRRRHI |
| | | VRKRTLRRLLQERELVEPLIPSGEAPNQALLRILKETEFKKIKVL |
| | | GSGAFGTVYKGLWIPEGEKVKIPVAIKELREATSPKANKEILDEA |
| | | YVMASVDNPHVCRLLGICLTSTVQLITQLMPFGCLLDYVREHKDN |
| | | IGSQYLLNWCVQIAKGMNYLEDRRLVHRDLAARNVLVKTPQHVKI |
| | | TDFGLAKLLGAEEKEYHAEGGKVPIKWMALESILHRIYTHQSDVW |
| | | SYGVTVWELMTFGSKPYDGIPASEISSILEKGERLPQPPICTIDV |
| | | YMIMVKCWMIDADSRPKFRELIIEFSKMARDPQRYLVIQGDERMH |
| | | LPSPTDSNEYRALMDEEDMDDVVDADEYLIPQQGFFSSPSTSRTP |
| | | LLSSLSATSNNSTVACIDRNGLQSCPIKEDSFLQRYSSDPTGALT |
| | | EDSIDDTFLPVPEYINQSVPKRPAGSVQNPVYHNQPLNPAPSRDP |
| | | HYQDPHSTAVGNPEYLNTVQPTCVNSTFDSPAHWAQKGSHQISLD |
| | | NPDYQQDFFPKEAKPNGIEKGSTAENAEYLRVAPQSSEFIGA |
| HER2/NEU | 17 | MELAALCRWGLLLALLPPGAASTQVCTGTDMKLRLPASPETHL |
| | | DMLRHLYQGCQVVQGNLELTYLPTNASLSFLQDIQEVQGYVLIAH |
| | | NQVRQVPLQRLRIVRGTQLFEDNYALAVLDNGDPLNNTTPVTGAS |
| | | PGGLRELQLRSLTEILKGGVLIQRNPQLCYQDTILWKDIFHKNNQ |
| | | LALTLIDTNRSRACHPCSPMCKGSRCWGESSEDCQSLTRTVCAGG |
| | | CARCKGPLPTDCCHEQCAAGCTGPKHSDCLACLHFNHSGICELHC |
| | | PALVTYNTDTFESMPNPEGRYTFGASCVTACPYNYLSTDVGSCTL |
| | | VCPLHNQEVTAEDGTQRCEKCSKPCARVCYGLGMEHLREVRAVTS |
| | | ANIQEFAGCKKIFGSLAFLPESFDGDPASNTAPLQPEQLQVFETL |
| | | EEITGYLYISAWPDSLPDLSVFQNLQVIRGRILHNGAYSLTLQGL |
| | | GISWLGLRSLRELGSGLALIHHNTHLCFVHTVPWDQLFRNPHQAL |
| | | LHTANRPEDECVGEGLACHQLCARGHCWGPGPTQCVNCSQFLRGQ |
| | | ECVEECRVLQGLPREYVNARHCLPCHPECQPQNGSVTCFGPEADQ |
| | | CVACAHYKDPPFCVARCPSGVKPDLSYMPIWKFPDEEGACQPCPI |
| | | NCTHSCVDLDDKGCPAEQRASPLTSIISAVVGILLVVVLGVVFGI |
| | | LIKRRQQKIRKYTMRRLLQETELVEPLTPSGAMPNQAQMRILKET |
| | | ELRKVKVLGSGAFGTVYKGIWIPDGENVKIPVAIKVLRENTSPKA |
| | | NKEILDEAYVMAGVGSPYVSRLLGICLTSTVQLVTQLMPYGCLLD |
| | | HVRENRGRLGSQDLLNWCMQIAKGMSYLEDVRLVHRDLAARNVLV |
| | | KSPNHVKITDFGLARLLDIDETEYHADGGKVPIKWMALESILRRR |
| | | FTHQSDVWSYGVTVWELMTFGAKPYDGIPAREIPDLLEKGERLPQ |
| | | PPICTIDVYMIMVKCWMIDSECRPRFRELVSEFSRMARDPQRFVV |
| | | IQNEDLGPASPLDSTFYRSLLEDDDMGDLVDAEEYLVPQQGFFCP |
| | | DPAPGAGGMVHHRHRSSSTRSGGGDLTLGLEPSEEEAPRSPLAPS |
| | | EGAGSDVFDGDLGMGAAKGLQSLPTHDPSPLQRYSEDPTVPLPSE |
| | | TDGYVAPLTCSPQPEYVNQPDVRPQPPSPREGPLPAARPAGATLE |
| | | RPKTLSPGKNGVVKDVFAFGGAVENPEYLTPQGGAAPQPHPPPAF |
| | | SPAFDNLYYWDQDPPERGAPPSTFKGTPTAENPEYLGLDVPV |
| MUC1 | 18 | MTPGTQSPFFLLLLLTVLTVVTGSGHASSTPGGEKETSATQRS |
| | | SVPSSTEKNAVSMTSSVLSSHSPGSGSSTTQGQDVTLAPATEPAS |
| | | GSAATWGQDVTSVPVTRPALGSTTPPAHDVTSAPDNKPAPGSTAP |
| | | PAHGVTSAPDTRPAPGSTAPPAHGVTSAPDTRPAPGSTAPPAHGV |
| | | TSAPDTRPAPGSTAPPAHGVTSAPDTRPAPGSTAPPAHGVTSAPD |
| | | TRPAPGSTAPPAHGVTSAPDTRPAPGSTAPPAHGVTSAPDTRPAP |
| | | GSTAPPAHGVTSAPDTRPAPGSTAPPAHGVTSAPDTRPAPGSTAP |
| | | PAHGVTSAPDTRPAPGSTAPPAHGVTSAPDTRPAPGSTAPPAHGV |
| | | TSAPDTRPAPGSTAPPAHGVTSAPDTRPAPGSTAPPAHGVTSAPD |
| | | TRPAPGSTAPPAHGVTSAPDTRPAPGSTAPPAHGVTSAPDTRPAP |
| | | GSTAPPAHGVTSAPDTRPAPGSTAPPAHGVTSAPDTRPAPGSTAP |
| | | PAHGVTSAPDTRPAPGSTAPPAHGVTSAPDTRPAPGSTAPPAHGV |
| | | TSAPDTRPAPGSTAPPAHGVTSAPDTRPAPGSTAPPAHGVTSAPD |
| | | TRPAPGSTAPPAHGVTSAPDTRPAPGSTAPPAHGVTSAPDTRPAP |
| | | GSTAPPAHGVTSAPDTRPAPGSTAPPAHGVTSAPDTRPAPGSTAP |
| | | PAHGVTSAPDTRPAPGSTAPPAHGVTSAPDTRPAPGSTAPPAHGV |
| | | TSAPDTRPAPGSTAPPAHGVTSAPDTRPAPGSTAPPAHGVTSAPD |
| | | TRPAPGSTAPPAHGVTSAPDTRPAPGSTAPPAHGVTSAPDTRPAP |
| | | GSTAPPAHGVTSAPDTRPAPGSTAPPAHGVTSAPDTRPAPGSTAP |
| | | PAHGVTSAPDTRPAPGSTAPPAHGVTSAPDTRPAPGSTAPPAHGV |
| | | TSAPDTRPAPGSTAPPAHGVTSAPDTRPAPGSTAPPAHGVTSAPD |
| | | NRPALGSTAPPVHNVTSASGSASGSASTLVHNGTSARATTTPASK |
| | | STPFSIPSHHSDTPTTLASHSTKTDASSTHHSSVPPLTSSNHSTS |
| | | PQLSTGVSFEELSFHISNLQFNSSLEDPSTDYYQELQRDISEMFL |
| | | QIYKQGGFLGLSNIKFRPGSVVVQLTLAFREGTINVHDVETQFNQ |
| | | YKTEAASRYNLTISDVSVSDVPFPFSAQSGAGVPGWGIALLVLVC |
| | | VLVALAIVYLIALAVCQCRRKNYGQLDIFPARDTYHPMSEYPTYH |
| | | THGRYVPPSSTDRSPYEKVSAGNGGSSLSYTNPAVAATSANL |
| EPCAM | 19 | MAPPQVLAFGLLLAAATATFAAAQEECVCENYKLAVNCFVNNN |
| | | RQCQCTSVGAQNTVICSKLAAKCLVMKAEMNGSKLGRRAKPEGAL |
| | | QNNDGLYDPDCDESGLFKAKQCNGTSMCWCVNTAGVRRTDKDTEI |
| | | TCSERVRTYWIIIELKHKAREKPYDSKSLRTALQKEITTRYQLDP |
| | | KFITSILYENNVITIDLVQNSSQKTQNDVDIADVAYYFEKDVKGE |
| | | SLFHSKKMDLTVNGEQLDLDPGQTLIYYVDEKAPEFSMQGLKAGV |
| | | IAVIVVVVIAVVAGIVVLVISRKKRMAKYEKAEIKEMGEMHRELN |
| | | A |
| PSMA | 20 | MFRNQYDNDVTVWSPQGRIHQIEYAMEAVKQGSATVGLKSKTH |
| | | AVLVALKRAQSELAAHQKKILHVDNHIGISIAGLTADARLLCNFM |
| | | RQECLDSRFVFDRPLPVSRLVSLIGSKTQIPTQRYGRRPYGVGLL |
| | | IAGYDDMGPHIFQTCPSANYFDCRAMSIGARSQSARTYLERHMSE |
| | | FMECNLNELVKHGLRALRETLPAEQDLTTKNVSIGIVGKDLEFTI |
| | | YDDDDVSPFLEGLEERPQRKAQPAQPADEPAEKADEPMEH |
| SLAMF7 | 21 | MAGSPTCLTLTYILWQLTGSAASGPVKELVGSVGGAVTFPLKS |
| | | KVKQVDSIVWTENTTPLVTIQPEGGTIIVTQNRNRERVDFPDGGY |
| | | SLKLSKLKKNDSGIYYVGIYSSSLQQPSTQEYVLHVYEHLSKPKV |
| | | TMGLQSNKNGTCVTNLTCCMEHGEEDVIYTWKALGQAANESHNGS |
| | | ILPISWRWGESDMTFICVARNPVSRNFSSPILARKLCEGAADDPD |
| | | SSMVLLCLLLVPLLLSLEVLGLFLWELKRERQEEYIEEKKRVDIC |
| | | RETPNICPHSGENTEYDTIPHTNRTILKEDPANTVYSTVEIPKKM |
| | | ENPHSLLTMPDTPRLFAYENVI |
| GPC3 | 22 | MAGTVRTACLVVAMLLSLDFPGQAQPPPPPPDATCHQVRSFFQ |
| | | RLQPGLKWVPETPVPGSDLQVCLPKGPTCCSRKMEEKYQLTARLN |
| | | MEQLLQSASMELKFLIIQNAAVFQEAFEIVVRHAKNYTNAMFKNN |
| | | YPSLTPQAFEFVGEFFTDVSLYILGSDINVDDMVNELFDSLFPVI |
| | | YTQLMNPGLPDSALDINECLRGARRDLKVEGNFPKLIMTQVSKSL |
| | | QVTRIFLQALNLGIEVINTTDHLKFSKDCGRMLTRMWYCSYCQGL |
| | | MMVKPCGGYCNVVMQGCMAGVVEIDKYWREYILSLEELVNGMYRI |
| | | YDMENVLLGLFSTIHDSIQYVQKNAGKLTTTIGKLCAHSQQRQYR |
| | | SAYYPEDLFIDKKVLKVAHVEHEETLSSRRRELIQKLKSFISFYS |
| | | ALPGYICSHSPVAENDTLCWNGQELVERYSQKAARNGMKNQFNLH |
| | | ELKMKGPEPVVSQIIDKLKHINQLLRTMSMPKGRVLDKNLDEEGF |
| | | ESGDCGDDEDECIGGSGDGMIKVKNQLRFLAELAYDLDVDDAPGN |
| | | SQQATPKDNEISTFHNLGNVHSPLKLLTSMAISVVCFFFLVH |
| PD-L1 | 23 | MRIFAVFIFMTYWHLLNAFTVTVPKDLYVVEYGSNMTIECKFP |
| | | VEKQLDLAALIVYWEMEDKNIIQFVHGEEDLKVQHSSYRQRARLL |
| | | KDQLSLGNAALQITDVKLQDAGVYRCMISYGGADYKRITVKVNAP |
| | | YNKINQRILVVDPVTSEHELTCQAEGYPKAEVIWTSSDHQVLSGK |
| | | TTTTNSKREEKLFNVTSTLRINTTTNEIFYCTFRRLDPEENHTAE |
| | | LVIPELPLAHPPNERTHLVILGAILLCLGVALTFIFRLRKGRMMD |
| | | VKKCGIQDTNSKKQSDTHLEET |
| NY-ESO-1 | 24 | MQAEGRGTGGSTGDADGPGGPGIPDGPGGNAGGPGEAGATGGR |
| | | GPRGAGAARASGPGGGAPRGPHGGAASGLNGCCRCGARGPESRLL |
| | | EFYLAMPFATPMEAELARRSLAQDAPPLPVPGVLLKEFTVSGNIL |
| | | TIRLTAADHRQLQLSISSCLQQLSLLMWITQCFLPVFLAQPPSGQ |
| | | RR |
| WT1 | 25 | MGSDVRDLNALLPAVPSLGGGGGCALPVSGAAQWAPVLDFAPP |
| | | GASAYGSLGGPAPPPAPPPPPPPPPHSFIKQEPSWGGAEPHEEQC |
| | | LSAFTVHFSGQFTGTAGACRYGPFGPPPPSQASSGQARMFPNAPY |
| | | LPSCLESQPAIRNQGYSTVTFDGTPSYGHTPSHHAAQFPNHSFKH |
| | | EDPMGQQGSLGEQQYSVPPPVYGCHTPTDSCTGSQALLLRTPYSS |
| | | DNLYQMTSQLECMTWNQMNLGATLKGVAAGSSSSVKWTEGQSNHS |
| | | TGYESDNHTTPILCGAQYRIHTHGVFRGIQDVRRVPGVAPTLVRS |
| | | ASETSEKRPFMCAYPGCNKRYFKLSHLQMHSRKHTGEKPYQCDFK |
| | | DCERRFSRSDQLKRHQRRHTGVKPFQCKTCQRKFSRSDHLKTHTR |
| | | THTGKTSEKPFSCRWPSCQKKFARSDELVRHHNMHQRNMTKLQLA |
| | | L |
| MAGE-A3 | 26 | MPLEQRSQHCKPEEGLEARGEALGLVGAQAPATEEQEAASSSS |
| | | TLVEVTLGEVPAAESPDPPQSPQGASSLPTTMNYPLWSQSYEDSS |
| | | NQEEEGPSTFPDLESEFQAALSRKVAELVHFLLLKYRAREPVTKA |
| | | EMLGSVVGNWQYFFPVIFSKASSSLQLVEGIELMEVDPIGHLYIF |
| | | ATCLGLSYDGLLGDNQIMPKAGLLIIVLAITAREGDCAPEEKIWE |
| | | ELSVLEVFEGREDSILGDPKKLLTQHFVQENYLEYRQVPGSDPAC |
| | | YEFLWGPRALVETSYVKVLHHMVKISGGPHISYPPLHEWVLREGE |
| | | E |
| AFP | 27 | MKWVESIFLIFLLNFTESRTLHRNEYGIASILDSYQCTAEISL |
| | | ADLATIFFAQFVQEATYKEVSKMVKDALTAIEKPTGDEQSSGCLE |
| | | NQLPAFLEELCHEKEILEKYGHSDCCSQSEEGRHNCFLAHKKPTP |
| | | ASIPLFQVPEPVTSCEAYEEDRETFMNKFIYEIARRHPFLYAPTI |
| | | LLWAARYDKIIPSCCKAENAVECFQTKAATVTKELRESSLLNQHA |
| | | CAVMKNEGTRTFQAITVTKLSQKFTKVNFTEIQKLVLDVAHVHEH |
| | | CCRGDVLDCLQDGEKIMSYICSQQDTLSNKITECCKLTTLERGQC |
| | | IIHAENDEKPEGLSPNLNRFLGDRDENQFSSGEKNIFLASFVHEY |
| | | SRRHPQLAVSVILRVAKGYQELLEKCFQTENPLECQDKGEEELQK |
| | | YIQESQALAKRSCGLFQKLGEYYLQNAFLVAYTKKAPQLTSSELM |
| | | AITRKMAATAATCCQLSEDKLLACGEGAADIIIGHLCIRHEMTPV |
| | | NPGVGQCCTSSYANRRPCFSSLVVDETYVPPAFSDDKFIFHKDLC |
| | | QAQGVALQTMKQEFLINLVKQKPQITEEQLEAVIADFSGLLEKCC |
| | | QGQEQEVCFAEEGQKLISKTRAALGV |
| PRAME | 28 | MERRRLWGSIQSRYISMSVWTSPRRLVELAGQSLLKDEALAIA |
| | | ALELLPRELFPPLFMAAFDGRHSQTLKAMVQAWPFTCLPLGVLMK |
| | | GQHLHLETFKAVLDGLDVLLAQEVRPRRWKLQVLDLRKNSHQDFW |
| | | TVWSGNRASLYSFPEPEAAQPMTKKRKVDGLSTEAEQPFIPVEVL |
| | | VDLFLKEGACDELFSYLIEKVKRKKNVLRLCCKKLKIFAMPMQDI |
| | | KMILKMVQLDSIEDLEVTCTWKLPTLAKFSPYLGQMINLRRLLLS |
| | | HIHASSYISPEKEEQYIAQFTSQFLSLQCLQALYVDSLFFLRGRL |
| | | DQLLRHVMNPLETLSITNCRLSEGDVMHLSQSPSVSQLSVLSLSG |
| | | VMLTDVSPEPLQALLERASATLQDLVFDECGITDDQLLALLPSLS |
| | | HCSQLTTLSFYGNSISISALQSLLQHLIGLSNLTHVLYPVPLESY |
| | | EDIHGTLHLERLAYLHARLRELLCELGRPSMVWLSANPCPHCGDR |
| | | TFYDPEPILCPCFMPN |
| - | 29 | SLLMWITQC |
| - | 30 | SLLMWITQV |
| - | 31 | RMFPNAPYL |
| - | 32 | KVAELVHFL |
| - | 33 | EVDPIGHLY |
| - | 34 | NYKRCFPVI |
| - | 35 | AAGIGILTV |
| - | 36 | YMLDLQPET |
| - | 37 | LKEFTVSGNILTIRL |
| - | 38 | LPVPGVLLKEFTVSGNILTI |
| - | 39 | TSYVKVLHHMVKISG |
| - | 40 | RNGYRALMDKSLHVGTQCALTRR |
| - | 41 | ALYVDSLFFL |

### Therapeutic Methods, Uses, and Compositions

The type III NKT cells (e.g., CD3⁺CD56⁺ type III NKT cells) described herein can be employed in various therapeutic and prophylactic applications. For instance, in some embodiments, the type III NKT cells described herein may be useful in treating or preventing a cancer. The type III NKT cells described herein may be administered per se or in any suitable pharmaceutical composition.

Accordingly, in certain aspects, the present disclosure provides methods of treating or preventing a cancer in a subject in need thereof by administering to the subject a therapeutically effective amount of type III NKT cells (e.g., CD3⁺CD56⁺ type III NKT cells). In certain aspects, the present disclosure further provides methods of preparing a therapy for treating or preventing a cancer in a subject in need thereof by: (a) isolating one or more type III NKT cells (e.g., CD3⁺CD56⁺ type III NKT cells) from a biological sample; and (b) culturing the one or more cells in a growth medium to produce an expanded cell population. In certain aspects, the present disclosure further provides methods of treating or preventing a cancer in a subject in need thereof by administering to the subject a therapeutically effective amount of an expanded cell population (e.g., an expanded cell population comprising type III NKT cells (e.g., CD3⁺CD56⁺ type III NKT cells), as described herein and/or prepared by a method described herein). Uses of the disclosed type III NKT cells, e.g., in treating or preventing a cancer, are also provided. Pharmaceutical compositions comprising a therapeutically effective amount of type III NKT cells (e.g., CD3⁺CD56⁺ type III NKT cells) are also disclosed, and are useful in the therapeutic methods and uses provided herein.

An exemplary embodiment is a method of treating or preventing a cancer in a subject in need thereof, comprising administering to the subject a therapeutically effective amount of type III NKT cells (e.g., CD3⁺CD56⁺ type III NKT cells), or a pharmaceutical composition comprising a therapeutically effective amount of type III NKT cells (e.g., CD3⁺CD56⁺ type III NKT cells) and at least one pharmaceutically acceptable carrier.

Another exemplary embodiment is a method of preparing a therapy for treating or preventing a cancer in a subject in need thereof, comprising: (a) isolating one or more type III NKT cells (e.g., CD3⁺CD56⁺ type III NKT cells) from a biological sample; and (b) culturing the one or more cells in a growth medium to produce an expanded cell population.

Another exemplary embodiment is a method of treating or preventing a cancer in a subject in need thereof, comprising administering to the subject a therapeutically effective amount of an expanded cell population (e.g., an expanded cell population comprising type III NKT cells (e.g., CD3⁺CD56⁺ type III NKT cells), as described herein and/or prepared by a method described herein), or a pharmaceutical composition comprising a therapeutically effective amount of an expanded cell population (e.g., an expanded cell population comprising type III NKT cells (e.g., CD3⁺CD56⁺ type III NKT cells), as described herein and/or prepared by a method described herein) and at least one pharmaceutically acceptable carrier.

Another exemplary embodiment is isolated type III NKT cells (e.g., CD3⁺CD56⁺ type III NKT cells) for use in treating or preventing a cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated type III NKT cells (e.g., CD3⁺CD56⁺ type III NKT cells) in treating or preventing a cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated type III NKT cells (e.g., CD3⁺CD56⁺ type III NKT cells) in the manufacture of a medicament for treating or preventing a cancer in a subject in need thereof. In some embodiments, the medicament comprises a therapeutically effective amount of the cells, or a pharmaceutical composition comprising a therapeutically effective amount of the cells and at least one pharmaceutically acceptable carrier.

As used herein, the term “treat” and its cognates refer to an amelioration of a disease, disorder, or condition (e.g., a cancer), or at least one discernible symptom thereof. The term “treat” encompasses but is not limited to complete treatment or complete amelioration of one or more symptoms of a cancer. In some embodiments, “treat” refers to at least partial amelioration of at least one measurable physical parameter, not necessarily discernible by the subject. In some embodiments, “treat” refers to inhibiting the progression of a disease, disorder, or condition, either physically (e.g., stabilization of a discernible symptom), physiologically (e.g., stabilization of a physical parameter), or both. In some embodiments, “treat” refers to slowing the progression or reversing the progression of a disease, disorder, or condition. As used herein, “treat” and its cognates also encompass delaying the onset or reducing the risk of acquiring a given disease, disorder, or condition.

In some embodiments, “treat” refers to administering to a subject suspected of having a disease, disorder, or condition (e.g., a cancer or a precancerous condition) a type III NKT cell, cell population, or composition disclosed herein. In some embodiments, a subject and/or a sample from a subject suspected of having a cancer and/or a precancerous condition may comprise one or more cells that are abnormal, malignant, and/or premalignant.

The terms “subject” and “patient” are used interchangeably herein to refer to any human or non-human animal in need of treatment. Non-human animals include all vertebrates (e.g., mammals and non-mammals). Non-limiting examples of mammals include humans, mice, rats, rabbits, dogs, monkeys, and pigs. In some embodiments, the subject is a human.

The term “donor,” as used herein, refers to any human or non-human animal that donates a biological sample (e.g., a blood sample) for use in a subject in need of treatment and/or for use in preparing a therapy (e.g., a type III NKT cell therapy disclosed herein) for a subject in need of treatment. In some embodiments, the donor is a human.

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Cancer cells can be in the form of a tumor or mass, but such cells may exist alone within a subject, or may circulate in the blood stream as independent cells, such as leukemia or lymphoma cells. The term “cancer” includes all types of cancers and cancer metastases, including hematological malignancies, solid tumors, sarcomas, carcinomas, and other solid and non-solid tumor cancers. In some embodiments, a cancer is a B-cell malignancy, leukemia, lymphoma, myeloma, or melanoma. In some embodiments, a cancer is acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma. In some embodiments, a cancer is AML. In some embodiments, a cancer is a refractory or relapsed cancer (e.g., refractory or relapsed AML). In some embodiments, a cancer is refractory or relapsed AML. In some embodiments, a cancer expresses a target antigen.

The term “target antigen,” as used herein, refers to any antigen targeted by a type III NKT cell and/or by a construct expressed by a type III NKT cell (e.g., a CAR, TCR, or TCRm). As used herein, the term “antigen” is synonymous with “antigenic determinant” and “epitope,” and refers to a site (e.g., a contiguous stretch of amino acids or a conformational configuration made up of different regions of non-contiguous amino acids) on a polypeptide macromolecule to which an antigen binding moiety (e.g., an antigen binding moiety of a CAR, TCR, or TCRm) binds, forming an antigen binding moiety-antigen complex. Useful antigenic determinants can be found, for example, within or on the surfaces of cancer cells, within or on the surfaces of virus-infected cells, within or on the surfaces of other diseased cells, free in blood serum, and/or in the extracellular matrix (ECM).

Exemplary target antigens are disclosed herein, and include without limitation CD19, IGF1R, ROR1, BCMA, CD123, CD33, CD38, CD138, CLL-1, LILRB4, GD2, CD20, CD22, CD30, MSLN, EGFRvIII, EGFR, HER2, MUC1, EPCAM, PSMA, SLAMF7, GPC3, PD-L1, NY-ESO-1, WT1, and MAGE-A3. A target antigen may include a full-length antigen (e.g., any of the exemplary antigens disclosed herein), as well as any form of the antigen that may result from cellular processing. A target antigen also encompasses functional variants or fragments of an antigen (e.g., any of the exemplary antigens disclosed herein), including but not limited to splice variants, allelic variants, and isoforms that retain one or more biologic functions of the antigen (i.e., variants and fragments are encompassed unless the context indicates that the term is used to refer to the wild-type antigen only). In some embodiments, a target antigen is a functional fragment of a full-length antigen.

In some embodiments of the methods and uses described herein, the target antigen is CD19. In some embodiments, an NKT cell and/or antigen binding portion (e.g., an antigen binding portion of a CAR, TCR, or TCRm expressed by an NKT cell) is capable of binding to CD19. In some embodiments, an NKT cell or related composition is useful for treating or preventing a CD19-expressing cancer. In some embodiments, the cancer is a B-cell malignancy. In some embodiments, the cancer is B-cell precursor acute lymphoblastic leukemia (B-ALL), non-Hodgkin lymphoma (NHL), or chronic lymphocytic leukemia (CLL). In some embodiments, the cancer is refractory or in relapse.

In some embodiments of the methods and uses described herein, the target antigen is ROR1. In some embodiments, an NKT cell and/or antigen binding portion (e.g., an antigen binding portion of a CAR, TCR, or TCRm expressed by an NKT cell) is capable of binding to ROR1. In some embodiments, an NKT cell or related composition is useful for treating or preventing a ROR1-expressing cancer. In some embodiments, the cancer is Ewing sarcoma, osteosarcoma, fibrosarcoma, rhabdomyosarcoma, chronic lymphocytic leukemia, mantle cell carcinoma, breast cancer, lung adenocarcinoma, melanoma, or ovarian cancer. In some embodiments, the cancer is refractory or in relapse.

In some embodiments of the methods and uses described herein, the target antigen is BCMA. In some embodiments, an NKT cell and/or antigen binding portion (e.g., an antigen binding portion of a CAR, TCR, or TCRm expressed by an NKT cell) is capable of binding to BCMA. In some embodiments, an NKT cell or related composition is useful for treating or preventing a BCMA-expressing cancer. In some embodiments, the cancer is multiple myeloma. In some embodiments, the cancer is refractory or in relapse (e.g., refractory or relapsed multiple myeloma).

In some embodiments of the methods and uses described herein, the target antigen is CD123, CD33, CD38, CD138, CLL-1, or LILRB4. In some embodiments, an NKT cell and/or antigen binding portion (e.g., an antigen binding portion of a CAR, TCR, or TCRm expressed by an NKT cell) is capable of binding to CD123, CD33, CD38, CD138, CLL-1, or LILRB4. In some embodiments, an NKT cell or related composition is useful for treating or preventing a CD123-expressing cancer. In some embodiments, an NKT cell or related composition is useful for treating or preventing a CD33-expressing cancer. In some embodiments, an NKT cell or related composition is useful for treating or preventing a CD38-expressing cancer. In some embodiments, an NKT cell or related composition is useful for treating or preventing a CD138-expressing cancer. In some embodiments, an NKT cell or related composition is useful for treating or preventing a CLL-1-expressing cancer. In some embodiments, an NKT cell or related composition is useful for treating or preventing a LILRB4-expressing cancer. In some embodiments, the cancer is AML. In some embodiments, the cancer is refractory or in relapse (e.g., refractory or relapsed AML). In some embodiments, the cancer is relapsed AML, e.g., after hematopoietic stem cell transplantation.

In some embodiments of the methods and uses described herein, the target antigen is EGFRvIII or EGFR. In some embodiments, an NKT cell and/or antigen binding portion (e.g., an antigen binding portion of a CAR, TCR, or TCRm expressed by an NKT cell) is capable of binding to EGFRvIII or EGFR. In some embodiments, an NKT cell or related composition is useful for treating or preventing a EGFRvIII-expressing cancer. In some embodiments, an NKT cell or related composition is useful for treating or preventing a EGFR-expressing cancer. In some embodiments, the cancer is glioblastoma. In some embodiments, the cancer is refractory or in relapse.

In some embodiments of the methods and uses described herein, the target antigen is GPC3. In some embodiments, an NKT cell and/or antigen binding portion (e.g., an antigen binding portion of a CAR, TCR, or TCRm expressed by an NKT cell) is capable of binding to GPC3. In some embodiments, an NKT cell or related composition is useful for treating or preventing a GPC3-expressing cancer. In some embodiments, the cancer is hepatocellular carcinoma. In some embodiments, the cancer is refractory or in relapse.

In some embodiments of the methods and uses described herein, the target antigen is NY-ESO-1. In some embodiments, NY-ESO-1 is presented by a HLA molecule, e.g., a HLA-A*0201 molecule. In some embodiments, an NKT cell and/or antigen binding portion (e.g., an antigen binding portion of a CAR, TCR, or TCRm expressed by an NKT cell) is capable of binding to NY-ESO-1. In some embodiments, an NKT cell or related composition is useful for treating or preventing a NY-ESO-1-expressing cancer. In some embodiments, the cancer is neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, or breast cancer. In some embodiments, the cancer is refractory or in relapse.

In some embodiments of the methods and uses described herein, the target antigen is WT1. In some embodiments, WT1 is presented by a HLA molecule, e.g., a HLA-A2 molecule. In some embodiments, an NKT cell and/or antigen binding portion (e.g., an antigen binding portion of a CAR, TCR, or TCRm expressed by an NKT cell) is capable of binding to WT1. In some embodiments, an NKT cell or related composition is useful for treating or preventing a WT1-expressing cancer. In some embodiments, the cancer is AML. In some embodiments, the cancer is refractory or in relapse (e.g., refractory or relapsed AML). In some embodiments, the cancer is relapsed AML, e.g., after hematopoietic stem cell transplantation.

In some embodiments of the methods and uses described herein, the target antigen is IGF1R, GD2, CD20, CD22, CD30, MSLN, HER2, MUC1, EPCAM, PSMA, SLAMF7, PD-L1, or MAGE-A3. In some embodiments, an NKT cell and/or antigen binding domain (e.g., an antigen binding domain of a CAR or TCRm expressed by an NKT cell) is capable of binding to IGF1R, GD2, CD20, CD22, CD30, MSLN, HER2, MUC1, EPCAM, PSMA, SLAMF7, PD-L1, or MAGE-A3. In some embodiments, an NKT cell or related composition is useful for treating or preventing a IGF1R-expressing cancer. In some embodiments, an NKT cell or related composition is useful for treating or preventing a GD2-expressing cancer. In some embodiments, an NKT cell or related composition is useful for treating or preventing a CD20-expressing cancer. In some embodiments, an NKT cell or related composition is useful for treating or preventing a CD22-expressing cancer. In some embodiments, an NKT cell or related composition is useful for treating or preventing a CD30-expressing cancer. In some embodiments, an NKT cell or related composition is useful for treating or preventing a MSLN-expressing cancer. In some embodiments, an NKT cell or related composition is useful for treating or preventing a HER2-expressing cancer. In some embodiments, an NKT cell or related composition is useful for treating or preventing a MUC1-expressing cancer. In some embodiments, an NKT cell or related composition is useful for treating or preventing a EPCAM-expressing cancer. In some embodiments, an NKT cell or related composition is useful for treating or preventing a PSMA-expressing cancer. In some embodiments, an NKT cell or related composition is useful for treating or preventing a SLAMF7-expressing cancer. In some embodiments, an NKT cell or related composition is useful for treating or preventing a PD-L1-expressing cancer. In some embodiments, an NKT cell or related composition is useful for treating or preventing a MAGE-A3-expressing cancer. In some embodiments, the cancer is refractory or in relapse.

Exemplary target antigens also include composite antigens (e.g., a composite antigen comprising a peptide and a human leukocyte antigen (HLA) molecule). In some embodiments, a target antigen is a composite antigen comprising a peptide and a HLA molecule.

In some embodiments, the HLA molecule is a class I HLA molecule. In some embodiments, the HLA molecule is a class I HLA binding peptide. In some embodiments, a class I HLA binding peptide is about 9 or 10 amino acids in length. Exemplary class I HLA binding peptides are disclosed herein, and include without limitation: NY-ESO-1-derived HLA-A*0201 binding peptide SLLMWITQC (SEQ ID NO: 29); NY-ESO-1-derived HLA-A*0201 binding peptide SLLMWITQV (SEQ ID NO: 30); WT1-derived HLA-A*0201 binding peptide RMFPNAPYL (SEQ ID NO: 31); MAGE-A3-derived HLA-A*0201 binding peptide KVAELVHFL (SEQ ID NO: 32); MAGE-A3-derived HLA-A*0201 binding peptide EVDPIGHLY (SEQ ID NO: 33); MAGE-A4-derived HLA-A*2402 binding peptide NYKRCFPVI (SEQ ID NO: 34); MART-1-derived HLA-A*0201 binding peptide AAGIGILTV (SEQ ID NO: 35); and HPV E7-derived HLA-A*0201 binding peptide YMLDLQPET (SEQ ID NO: 36). In some embodiments, a HLA molecule comprises an amino acid sequence of SLLMWITQC (SEQ ID NO: 29); SLLMWITQV (SEQ ID NO: 30); RMFPNAPYL (SEQ ID NO: 31); KVAELVHFL (SEQ ID NO: 32); EVDPIGHLY (SEQ ID NO: 33); NYKRCFPVI (SEQ ID NO: 34); AAGIGILTV (SEQ ID NO: 35); and/or YMLDLQPET (SEQ ID NO: 36).

In some embodiments, the HLA molecule is a class II HLA molecule. In some embodiments, the HLA molecule is a class II HLA binding peptide. In some embodiments, a class II HLA binding peptide is about 13 to about 25 amino acids in length. Exemplary class II HLA binding peptides are disclosed herein, and include without limitation: NY-ESO-1-derived HLA-DRB1*0401 binding peptide LKEFTVSGNILTIRL (SEQ ID NO: 37); NY-ESO-1-derived HLA-DRB1*0401 binding peptide LPVPGVLLKEFTVSGNILTI (SEQ ID NO: 38); MAGE-A3-derived HLA-DRB1*1101 binding peptide TSYVKVLHHMVKISG (SEQ ID NO: 39); and MART-1-derived HLA-DRB1*0401 binding peptide RNGYRALMDKSLHVGTQCALTRR (SEQ ID NO: 40). In some embodiments, a HLA molecule comprises an amino acid sequence of LKEFTVSGNILTIRL (SEQ ID NO: 37); LPVPGVLLKEFTVSGNILTI (SEQ ID NO: 38); TSYVKVLHHMVKISG (SEQ ID NO: 39); and/or RNGYRALMDKSLHVGTQCALTRR (SEQ ID NO: 40).

In some embodiments, the peptide comprises an AFP peptide. In some embodiments of the methods and uses described herein, the composite antigen comprises an AFP peptide and a HLA molecule, e.g., a HLA-A2 molecule. In some embodiments, an NKT cell and/or antigen binding portion (e.g., an antigen binding portion of a CAR, TCR, or TCRm expressed by an NKT cell) is capable of binding to a composite antigen comprising an AFP peptide and a HLA molecule, e.g., a HLA-A2 molecule. In some embodiments, an NKT cell or related composition is useful for treating or preventing a composite antigen-expressing cancer. In some embodiments, the cancer is hepatocellular carcinoma. In some embodiments, the cancer is refractory or in relapse.

In some embodiments, the peptide comprises a PRAME peptide. In some embodiments, the PRAME peptide comprises an amino acid sequence of ALYVDSLFFL (SEQ ID NO: 41). In some embodiments of the methods and uses described herein, the composite antigen comprises a PRAME peptide and a HLA molecule, e.g., a HLA-A*0201 molecule. In some embodiments, an NKT cell and/or antigen binding portion (e.g., an antigen binding portion of a CAR, TCR, or TCRm expressed by an NKT cell) is capable of binding to a composite antigen comprising a PRAME peptide and a HLA molecule, e.g., a HLA-A*0201 molecule. In some embodiments, an NKT cell or related composition is useful for treating or preventing a composite antigen-expressing cancer. In some embodiments, the cancer is B-ALL, AML, multiple myeloma, T cell lymphoma, melanoma, non-small cell lung cancer, colon adenocarcinoma, or breast adenocarcinoma. In some embodiments, the cancer is refractory or in relapse.

In some embodiments, the peptide comprises a WT1 peptide. In some embodiments of the methods and uses described herein, the composite antigen comprises a WT1 peptide and a HLA molecule, e.g., a HLA-A2 molecule. In some embodiments, an NKT cell and/or antigen binding portion (e.g., an antigen binding portion of a CAR, TCR, or TCRm expressed by an NKT cell) is capable of binding to a composite antigen comprising a WT1 peptide and a HLA molecule, e.g., a HLA-A2 molecule. In some embodiments, an NKT cell or related composition is useful for treating or preventing a composite antigen-expressing cancer. In some embodiments, the cancer is AML. In some embodiments, the cancer is refractory or in relapse (e.g., refractory or relapsed AML). In some embodiments, the cancer is relapsed AML, e.g., after hematopoietic stem cell transplantation.

In some embodiments of the methods and uses described herein, the NKT cells are formulated and/or used as a pharmaceutical composition. Accordingly, in certain aspects, the present disclosure provides pharmaceutical compositions comprising isolated type III NKT cells (e.g., CD3⁺CD56⁺ type III NKT cells). An exemplary embodiment is a pharmaceutical composition, e.g., for treating or preventing a cancer in a subject in need thereof, comprising isolated type III NKT cells (e.g., CD3⁺CD56⁺ type III NKT cells). In some embodiments, a pharmaceutical composition further comprises at least one pharmaceutically acceptable carrier. Pharmaceutical compositions may also comprise one or more additional therapeutic agents that are suitable for treating or preventing, for example, a cancer (e.g., an anti-cancer agent, a standard-of-care agent for the particular cancer being treated, etc.). Pharmaceutical compositions may also comprise one or more inactive carriers, excipients, and/or stabilizer components, and the like. Methods of formulating pharmaceutical compositions and suitable formulations (e.g., for intravenous, systemic, or other modes of administration) are known in the art (see, e.g., “Remington's Pharmaceutical Sciences,” Mack Publishing Co., Easton, Pa.). Appropriate formulation may depend on the route of administration.

As used herein, a “pharmaceutical composition” refers to a preparation of an NKT cell or cell population (e.g., a CD3⁺CD56⁺ type III NKT cell or cell population) and, optionally, comprising one or more additional components suitable for administration to a subject, such as a physiologically acceptable carrier and/or excipient. The pharmaceutical compositions provided herein are in such form as to permit administration and subsequently provide the intended biological activity of the active ingredient(s) and/or to achieve a therapeutic effect. The pharmaceutical compositions provided herein contain no additional components which are unacceptably toxic to a subject to which the formulation would be administered.

As used herein, the phrases “pharmaceutically acceptable carrier” and “physiologically acceptable carrier,” which may be used interchangeably, refer to a carrier or a diluent that does not cause significant irritation to a subject and does not abrogate the biological activity and properties of the administered NKT cell or cell population or any additional therapeutic agent in the composition. Pharmaceutically acceptable carriers may enhance or stabilize the composition and/or can be used to facilitate preparation of the composition. Pharmaceutically acceptable carriers can include solvents, dispersion media, coatings, antibacterial and antifungal agents, isotonic and absorption delaying agents, and the like that are physiologically compatible. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20. Certain components included in a pharmaceutical composition of the present disclosure may be considered as a pharmaceutically acceptable carrier or an excipient.

A pharmaceutical composition of the present disclosure can be administered by a variety of methods known in the art. The route and/or mode of administration may vary depending upon the desired results. In some embodiments, the administration is intratumoral, intraventricular, intravenous, intramuscular, intraperitoneal, subcutaneous, parenteral, spinal, or epidermal. In some embodiments, the pharmaceutically acceptable carrier is suitable for intratumoral, intraventricular, intravenous, intramuscular, intraperitoneal, subcutaneous, parenteral, spinal, or epidermal administration (e.g., by injection or infusion).

An NKT cell or cell population (e.g., a CD3⁺CD56⁺ type III NKT cell or cell population) may be administered alone or in combination with at least one additional therapeutic agent (e.g., an anti-cancer agent, a standard-of-care agent for the particular cancer being treated, etc.), and may be administered in any acceptable formulation, dosage, or dosing regimen. When administered in combination with an additional therapeutic agent, the additional therapeutic agent may be administered according to its standard dosage and/or dosing regimen. Alternatively, the additional therapeutic agent may be administered at a higher or lower amount and/or frequency, as compared to its standard dosage and/or dosing regimen. In some embodiments, the additional therapeutic agent is administered at a lower amount and/or frequency.

As used herein, the term “agent” refers to a chemical compound, a mixture of chemical compounds, a biological macromolecule, or an extract made from biological materials. The term “therapeutic agent” refers to an agent that is capable of modulating a biological process and/or has biological activity. The NKT cells and cell populations described herein are exemplary therapeutic agents. Additional therapeutic agents (e.g., those which may be administered in combination with an NKT cell or cell population described herein) may comprise any active ingredients suitable for the particular indication being treated (e.g., a cancer), e.g., those with complementary activities that do not adversely affect each other.

Typically, a therapeutically effective dose of an NKT cell or cell population is employed in the pharmaceutical compositions of the present disclosure. The NKT cell or cell population may be formulated into a pharmaceutically acceptable dosage form by conventional methods known in the art.

Dosage regimens for an NKT cell or cell population alone or in combination with at least one additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally decreased or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier.

Dosage values for compositions comprising an NKT cell or cell population and/or any additional therapeutic agent(s), may be selected based on the unique characteristics of the active agent(s), and the particular therapeutic effect to be achieved. A physician or veterinarian can start doses of the NKT cell or cell population employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the subject, whether the subject is human or an animal, other medications administered, and whether treatment is prophylactic or therapeutic. The selected dosage level may also depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors. Treatment dosages may be titrated to optimize safety and efficacy.

As used herein, the terms “therapeutically effective dose” and “therapeutically effective amount” are used to refer to an amount sufficient to measurably decrease at least one symptom or measurable parameter associated with a medical condition or infirmity, to normalize body functions in a disease or disorder that results in the impairment of specific bodily functions, or to provide improvement in, or slow the progression of, one or more clinically measured parameters of a disease. A therapeutically effective amount may, for example, be sufficient to treat, prevent, reduce the severity, delay the onset, and/or reduce the risk of occurrence of one or more symptoms of a cancer. A therapeutically effective amount, as well as a therapeutically effective frequency of administration, can be determined by methods known in the art and discussed herein. In some embodiments of the compositions and methods described herein, an NKT cell or cell population is administered in an amount that is therapeutically effective when administered as a single agent. In some other embodiments, an NKT cell or cell population and at least one additional therapeutic agent is each administered in an amount that is therapeutically effective when the agents are used in combination. In some embodiments, a therapeutically effective amount of an NKT cell or cell population is the amount required to kill a cancer cell population or a portion thereof in a subject. In some embodiments, a therapeutically effective amount of an NKT cell or cell population is the amount required to reduce or slow the expansion of a cancer cell population in a subject. In some embodiments, a therapeutically effective amount of an NKT cell or cell population is the amount required to reduce or slow the growth of a tumor in a subject.

In some embodiments, a therapeutically effective amount of an NKT cell or cell population is about 1×10⁷ to about 5×10⁹ cells. In some embodiments, a therapeutically effective amount of an NKT cell or cell population is about 1×10⁷, about 2×10⁷, about 3×10⁷, about 4×10⁷, about 5×10⁷, about 6×10⁷, about 7×10⁷, about 8×10⁷, or about 9×10⁷ cells. In some embodiments, a therapeutically effective amount of an NKT cell or cell population is about 1×10⁹, about 2×10⁹, about 3×10⁹, about 4×10⁹, about 5×10⁹, about 6×10⁹, about 7×10⁹, about 8×10⁹, or about 9×10⁹ cells. In some embodiments, a therapeutically effective amount of an NKT cell or cell population is about 1×10⁹, about 2×10⁹, about 3×10⁹, about 4×10⁹, or about 5×10⁹ cells. In some embodiments, a therapeutically effective amount of an NKT cell or cell population is less than about 1×10⁷ cells. In some embodiments, a therapeutically effective amount of an NKT cell or cell population is more than about 5×10⁹ cells. In some embodiments, a cell, cell population, or pharmaceutical composition is administered to a subject on a single occasion. In some embodiments, a cell, cell population, or pharmaceutical composition is administered to a subject on multiple occasions (e.g., hourly, daily, weekly, bi-weekly, monthly, or yearly).

A therapeutically effective dose of an NKT cell or cell population described herein generally provides therapeutic benefit without causing substantial toxicity. Toxicity and therapeutic efficacy of an NKT cell or cell population can be determined by standard pharmaceutical procedures, e.g., in cell culture or in animal models. Cell culture assays and animal studies can be used to determine the LD50 (the dose lethal to 50% of a population) and the ED50 (the dose therapeutically effective in 50% of a population). The dose ratio between toxic and therapeutic effects is the therapeutic index, which can be expressed as the ratio LD50/ED50. In some embodiments, an NKT cell or cell population exhibits a high therapeutic index. The data obtained from cell culture assays and animal studies can be used in formulating a range of dosages suitable for use in humans. In some embodiments, the dosage lies within a range of circulating concentrations that include the ED50 with minimal or no toxicity. The dosage may vary within this range depending upon a variety of factors, e.g., the dosage form employed, the route of administration utilized, the condition of the subject, and the like. The exact formulation, route of administration, and dosage can be chosen by an attending physician in view of the subject's condition.

In some embodiments, an NKT cell, cell population, or pharmaceutical composition is administered on a single occasion. In some embodiments, an NKT cell, cell population, or pharmaceutical composition is administered on multiple occasions. Intervals between single dosages can be, e.g., hourly, daily, weekly, bi-weekly, monthly, or yearly. Intervals can also be irregular, based on measuring levels of the administered agent (e.g., an NKT cell or cell population) in the subject in order to maintain a relatively consistent concentration of the agent. The dosage and frequency of administration of an NKT cell or cell population may also vary depending on whether the treatment is prophylactic or therapeutic. In prophylactic applications, a relatively low dosage may be administered at relatively infrequent intervals over a long period of time. Some subjects may continue to receive treatment for the rest of their lives. In therapeutic applications, a relatively higher dosage at relatively shorter intervals is sometimes required until progression of the disease is reduced or terminated, and/or until the subject shows partial or complete amelioration of one or more symptoms of disease. Thereafter, the subject may be administered a lower, e.g., prophylactic, dosage regime.

In some embodiments, kits and articles of manufacture for use in the therapeutic and prophylactic applications described herein are also provided. In some embodiments, the present disclosure provides a kit or article of manufacture comprising an NKT cell or cell population. In some embodiments, the kit or article of manufacture further comprises one or more additional components, including but not limited to: instructions for use; other reagents, e.g., a therapeutic agent (e.g., an anti-cancer agent); devices, containers, or other materials for preparing the NKT cell or cell population for administration; pharmaceutically acceptable carriers; and devices, containers, or other materials for administering the NKT cell or cell population to a subject. Instructions for use can include guidance for therapeutic applications including suggested dosages and/or modes of administration, e.g., in a subject having or suspected of having a cancer. In some embodiments, the kit comprises an NKT cell or cell population, and instructions for use of the NKT cell or cell population in treating and/or preventing a cancer.

### EXAMPLES

The following examples provide illustrative embodiments of the disclosure. The examples provided do not in any way limit the disclosure.

### Materials and Methods

Cell Culture: K562 (erythroleukemia), K562CD19 (CD19 transfected K562), Daudi (B-cell Burkitt's lymphoma), Raji (B-cell Burkitt's lymphoma), Nalm-6 (B-cell precursor leukemia), HL-60 (acute myeloid leukemia, AML), HL-60mx (selected for Mitoxantrone resistance), KG-1 (AML), Molm-13 (AML), Molm-14 (AML), MV4:11 (AML), THP-1 (AML), U937 (AML), as well as their luciferase-expressing counterparts, in addition to SaOS2-hfflucN (osteosarcoma), Rh30-hfflucN (alveolar rhabdomyosarcoma), and TC71-hfflucN (Ewing sarcoma) cell lines, were maintained in RPMI 1640 medium supplemented with 10% heat-inactivated fetal bovine serum (FBS), 2 mM L-glutamine, 50 U/mL penicillin, and 50 μg/mL streptomycin. SaOS2-hfflucN, Rh30-hfflucN, and TC71-hfflucN were lentivirally transduced cell lines expressing humanized firefly luciferase and ΔNGFR, as previously described (Huang et al., Mol Ther. 2008; 16(3):580-9; Huang et al., PLoS ONE. 2015; 10 (7):e0133152).

DNT Cell Isolation and Culture: Double negative T cells (DNT) were isolated by depleting CD4⁺ and CD8⁺ cells from PBMCs using CD4 and CD8 depletion cocktail (Stemcell Technologies) and cultured in anti-CD3 antibody-coated plates (OKT3; 5 μg/mL) for 3 days in RPMI 1640 supplemented with 10% FBS, L-glutamine, 2-mercaptoethanol (2-ME), penicillin and streptomycin (human T cell medium), and 250 IU/mL IL-2 (Proleukin, Novartis Pharmaceuticals), as previously described (Lee et al., Clin Cancer Res. 2018; 24(2):370-82). On days 7, 10, and 14, soluble anti-CD3 (0.1 μg/mL) was added. On days 3, 7, and 10, fresh media and IL-2 were added. DNT were harvested 10 to 20 days post-expansion for subsequent experiments.

CD3⁺CD56⁺ NKT Isolation and Culture: NKT cells were isolated from PBMCs using a CD3⁺CD56⁺ NKT Cell Isolation Kit (Miltenyi Biotec, Cat No. 130-093-064). CD3⁺CD56⁺ NKT cells were cultured in a 24-well plate in human T cell medium with Dynabeads Human T-Activator CD3/CD28 (Invitrogen, Cat No. 11131D) for 3 days. After removal of CD3/CD28 beads, CD3⁺CD56⁺ NKT cells were cultured in human T cell medium supplemented with human IL-2 (50 IU/mL, Proleukin, Novartis Pharmaceuticals), IL-7 (10 ng/mL, Peprotech, Cat No. 200-07), and IL-15 (10 ng/mL, Peprotech, Cat No. 200-15). Expansion of CD3⁺CD56⁺ NKT cells was performed in T25 flasks using OKT3, as previously described (Zhou et al., Cancer Res. 2005; 65(3):1079-98). An alternative method of culture and expansion of NKT cells was carried out using initial OKT3-coated flasks or plates and subsequently soluble OKT3 as described in the section of DNT cell culture.

iNKT Cell Isolation and Culture: iNKT cells were isolated from PBMCs using anti-iNKT microbeads (Miltenyi Biotec, Cat No. 130-094-842) and cultured in a 48-well or 24-well plate in human T cell medium with irradiated negative fraction of PBMCs supplemented with α-GalCer (100 ng/mL, DiagnoCine, Cat No. KRN7000). Expansion of iNKT cells was carried out in a 24-well plate or in T25 flasks with irradiated PBMCs, α-GalCer (100 ng/mL), and IL-2 (50 IU/mL).

Lentiviral Production and Transduction: An HIV-1-based bidirectional vector expressing humanized firefly lucif erase and ANGFR (hfflucN) was constructed and lentivirally produced in 293T cells, as previously described (Huang et al., Mol Ther. 2008; 16(3):580-9; Huang et al., PLoS ONE. 2015; 10 (7):e0133152). A lentiviral vector expressing a CD19 CAR comprising a single chain variable region of an anti-CD19 antibody, a CD8a hinge and transmembrane region, and an intracellular domain of 4-1BB and a CD3 zeta chain was constructed based on a lentiviral vector previously described (Tammana et al., Hum Gene Ther. 2010; 21:75-86). Leukemia cell lines were spin transduced at 1170 g at 32° C. for 1 h with the hfflucN lentivirus in the presence of polybrene (8 μg/ml) and then sorted by FACS or enriched by anti-NGFR-biotin and anti-biotin microbeads (Miltenyi Biotec) for NGFR expression. All transduced cell lines were verified by flow cytometric analysis of NGFR expression and hffluc bioluminescence activity using a Synergy 2 microplate reader (BioTek). Lentiviral CD19 CAR transduction in T cells was carried out in a 6-well non tissue plate precoated with retronectin and spun at 1170 g at 32° C. for 2 h.

Luciferase-Based Killing Assay: Luciferase-expressing target cells (1×10⁴ in 50 μL per well) were incubated with 50 μL of T cells per well at different effector:target (E/T) ratios in quadruplicate in a 96-well flat-bottom white polystyrene microplate (Corning, Cat No. 3912) or in triplicate in 96-well U-bottom white polystyrene microplate (Corning, Cat No. 3789) when antibody blocking assays were performed. A spontaneous or maximal killing was set up by adding 50 μL per well of culture medium or 1% Triton X-100 instead of T cells, respectively. After a 4 h incubation at 37° C., 10 μL of D-luciferin (1:10 of 30 mg/mL stock) or 50 μL (1:50 of 30 mg/mL) was added to each well. Luciferase activity was measured using a Synergy 2 microplate reader (BioTek). Percent specific lysis was calculated as follows:

$$\% Specific lysis = ( spontaneous death R L U - sample R L U ) / ( spontaneous death R L U - maximal death R L U ) \times 100. R L U = Relative Luminescence Units .$$

Flow Cytometry: Flow cytometric analysis was performed on a Miltenyi MACSQuant or BD Accuri C6 or BD FACSCelesta cytometer. Data were analyzed with Flowjo software 7 or 10. Surface CAR expression was detected with biotinylated protein L (GenScript, Cat No. M00097) and streptavidin PE (BioLegend, Cat No. 405203).

CD107a Degranulation-Based Cytotoxicity and Intracellular INF-γ Staining Assay: Degranulation was measured during a 5-h co-culture of 5×10⁴ tumor cells and 1×10⁶ T cells by the addition of CD107α-PE-Cy7 or mIgG1, k-PE-Cy7. PMA (50 ng/mL) and ionomycin (1 μg/mL) were used as a positive control. After a 1-h incubation, Golgi Plus and Golgi Stop (BD Biosciences) were added to the culture. After an additional 4-h incubation, cells were washed once and stained with CD3-FITC, CD4-PE/CD8-PE, CD56-APC, or isotype controls at 4° C. for 20 min. Cells were washed and incubated in Cytofix/Cytoperm buffer (BD Biosciences) at 4° C. for 20 min and washed with Perm/Wash buffer. Cells were then stained with IFN-γ-Pac Blue or mIgG1, k-Pac Blue at 4° C. for 20 min, washed, and resuspended in Perm/Wash buffer. Cells were analyzed on a Miltenyi MACSQuant cytometer.

Cytokine Release Assays: Cytokine release assays were performed by co-culturing 1-5×10⁵ T cells with 2×10⁴ target cells per well in duplicate in 96-well round- or flat-bottom plates. After 24 h, supernatants were assayed using a IFN-γ ELISA kit (BioLegend, Cat No. 430108).

CD1d antibody blocking assays: Anti-human CD1d monoclonal antibodies (clone 42.1 from BD Biosciences and clone 51.1 from BioLegend) and isotype control antibodies were used in blocking CD1d-restricted presentation of α-GalCer antigen to iNKT cells (Ishihara et al. J Immunol. 2000; 165(3):1659-64; Mangan et al. J Immunol. 2013; 191(1):30-34; Jahnke et al. Front Immunol. 2019; 10:1542). Anti-human CD1d and isotype control antibodies at a concentration of 10-50 μg/mL were pre-incubated with α-GalCer antigen pulsed target cells for 1 h at 37° C. prior to addition of NKT cells in triplicate in a 4 h luciferase-based killing assay or a 24 h ELISA assay.

Genomic CD1d deletion using the CRISPR-Cas9 system: The RNA-guided Cas9 nuclease from the microbial clustered regularly interspaced short palindromic repeats (CRISPR) was used to knock out CD1d in AML cells (Ran et al. Nature Protocols. 2013; 8(11): 2281-08). Guide RNA1, 2, 3 and 4 was designed to target the first (gRNA1, 2 and 3) and second exon (gRNA4) of human CD1d. 20-base pair targeting oligonucleotides were subcloned in a lentiCRISPR v2 vector.

Statistical analysis: Data were calculated as the means±standard deviation (SD) and analyzed using a two-sample t-test (2 tailed, unequal variance) with a p value of <0.05 regarded as significant.

### Results

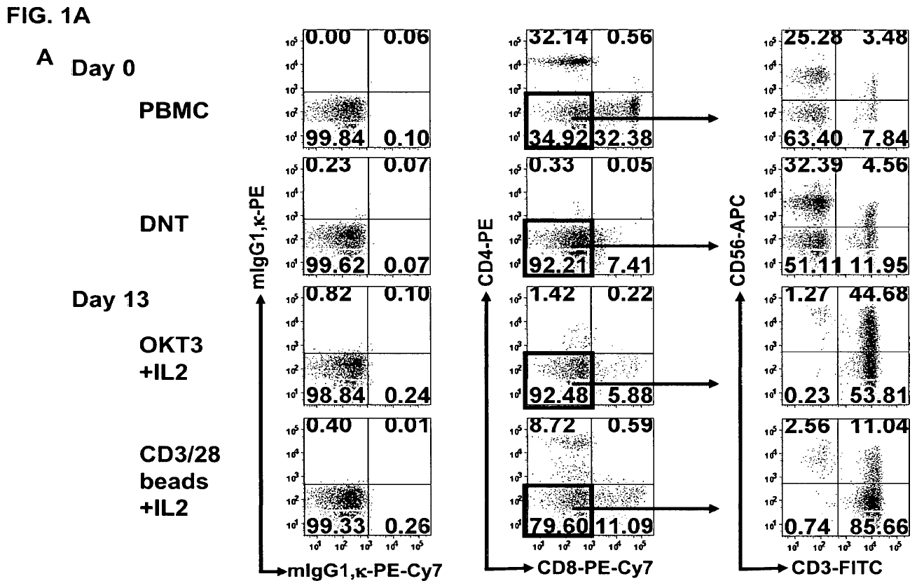

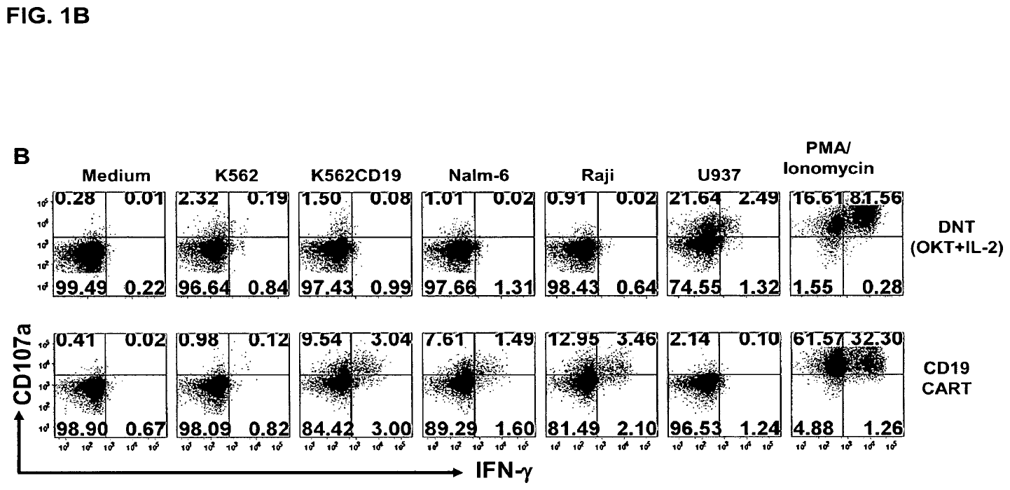

FIG. 1A shows that human CD4 and CD8 depletion cocktails depleted more than 90% of CD4 and CD8 T cells, with <10% of CD4⁺ and CD8⁺ T cells and about 15% of CD3⁺CD56⁺ and CD3⁻CD56⁺ cells remaining on day 0. After two-week cultures, DNT phenotypes were 95% of CD4⁻CD8⁻, 1.1% of CD4⁺, and 4.0% of CD8⁺ T cells. 44% of CD3⁺CD56⁺ NKT cells remained in CD4⁻CD8⁻ populations, as compared to 11% from the CD3/CD28 beads protocol.

FIG. 1B shows that 21.64% DNT cells upregulated expression of CD107a in response to U937 AML cells, as compared to <=2.32% DNT cells in response to K562 CML, CD19 transfected K562 (K562CD19), Nalm-6 B-ALL, and Raji B-NHL cells. DNT cells also specifically produced IFN-γ in response to U937 AML cells (2.49% versus <=0.19% of K562, K562CD19, Nalm-6 and Raji). In contrast, CD19 CAR-T cells specifically recognized B-cell tumor lines and K562CD19. DNT cultures with nearly 50% CD3⁺CD56⁺ NKT cells in CD4⁻CD8⁻ populations and ˜10% or higher of CD4⁺ and CD8⁺ T cells were generated. CD4⁺, CD8⁺ T cells, CD3⁻CD56⁺ NK cells, and CD3⁺CD56⁺ NKT cells were also expanded in parallel with DNT cells.

Flow cytometry-based CD107a assays generally use one effector:target (E/T) ratio to test T cell potency. A non-radioactive luciferase-based killing assay to test T cell potency using different E/T ratios was established.

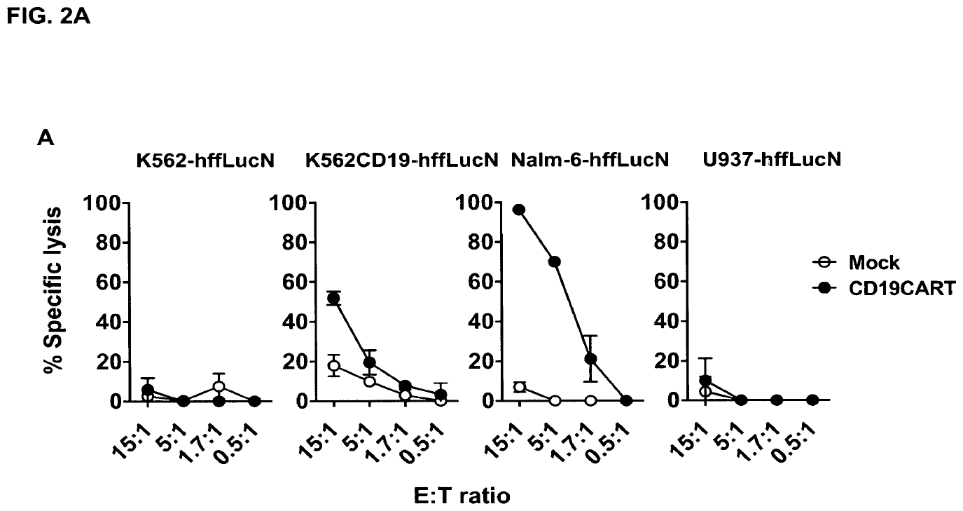

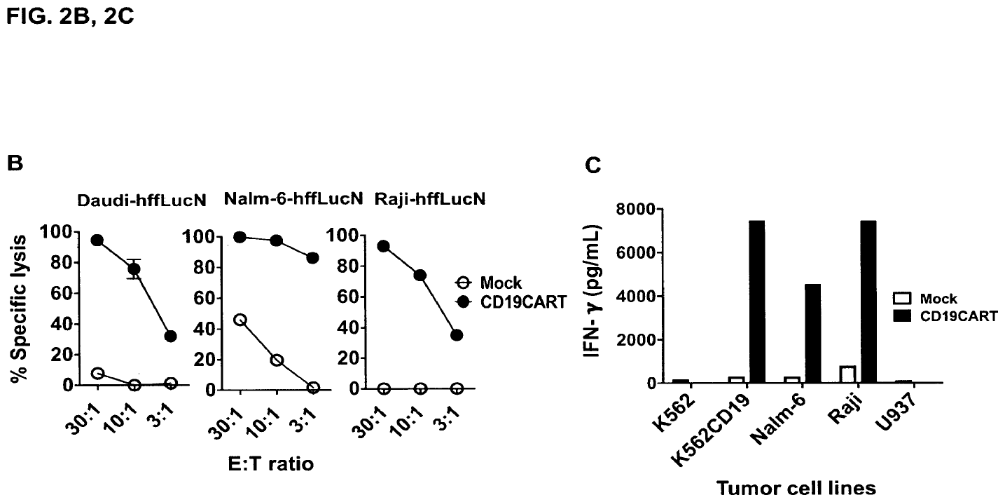

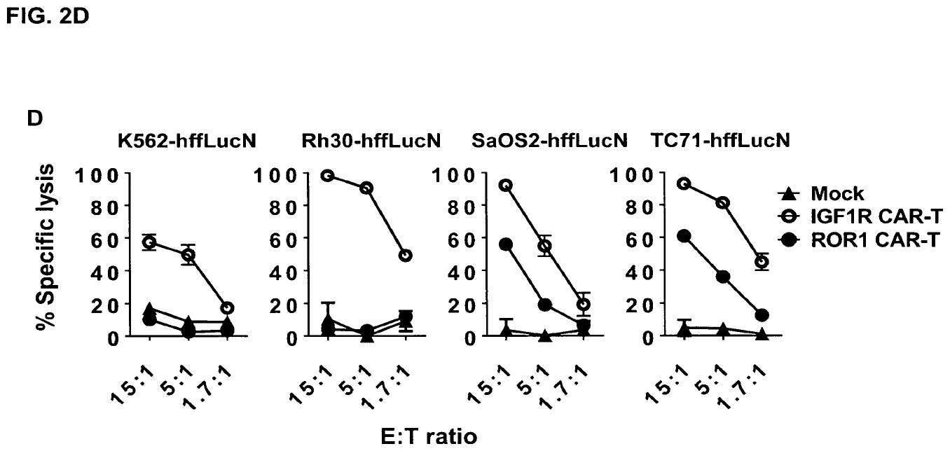

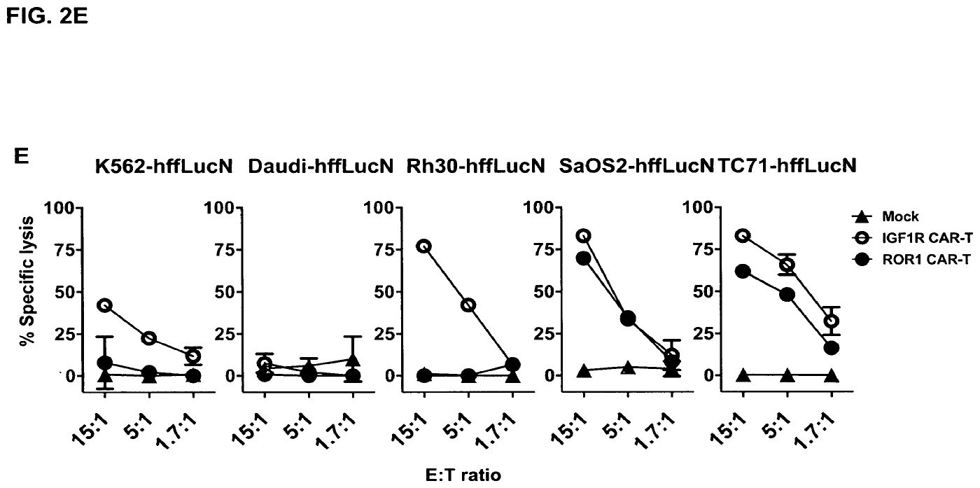

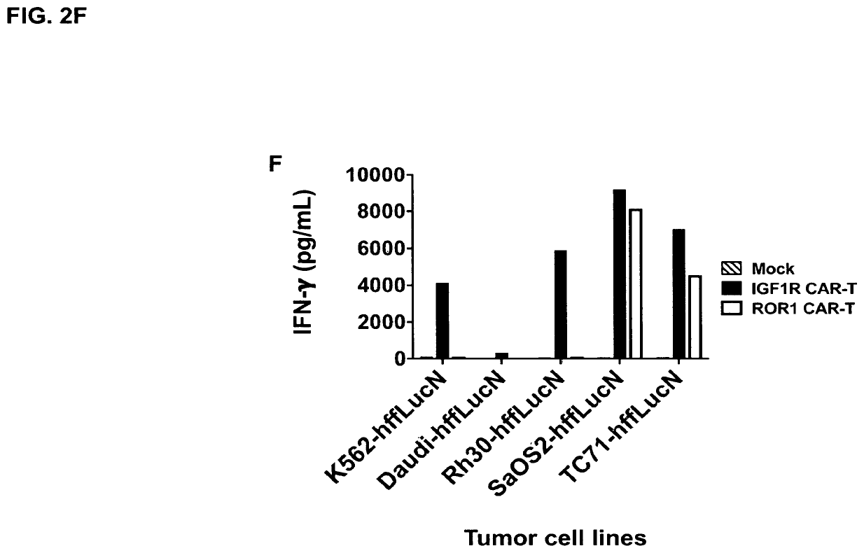

FIG. 2A and FIG. 2B show that CD19 CAR-T specifically killed CD19⁺ K562CD19, Nalm-6, Daudi, and Raji cells. Mock T cells used as a control did not kill target cells. The results from luciferase-based killing assays were consistent with those from IFN-γ release assays (FIG. 2C). Luciferase-based killing assays were also validated using insulin-like growth factor receptor (IGF1R) CAR-T and tyrosine kinase-like orphan receptor 1 (ROR1) CAR-T (Huang et al. Plos ONE. 2015; 10 (7):e0133152). FIG. 2D and FIG. 2E show that IGF1R CAR-T specifically killed IGF1R⁺ Rh30, SaOS2, TC71, and K562 cells, while ROR1 CAR-T specifically killed ROR1⁺ SaOS2 and TC71 cells. IFN-γ release assays also confirmed the antigen specific recognition of sarcoma cells by IGF1R CAR-T and ROR1 CAR-T (FIG. 2F).

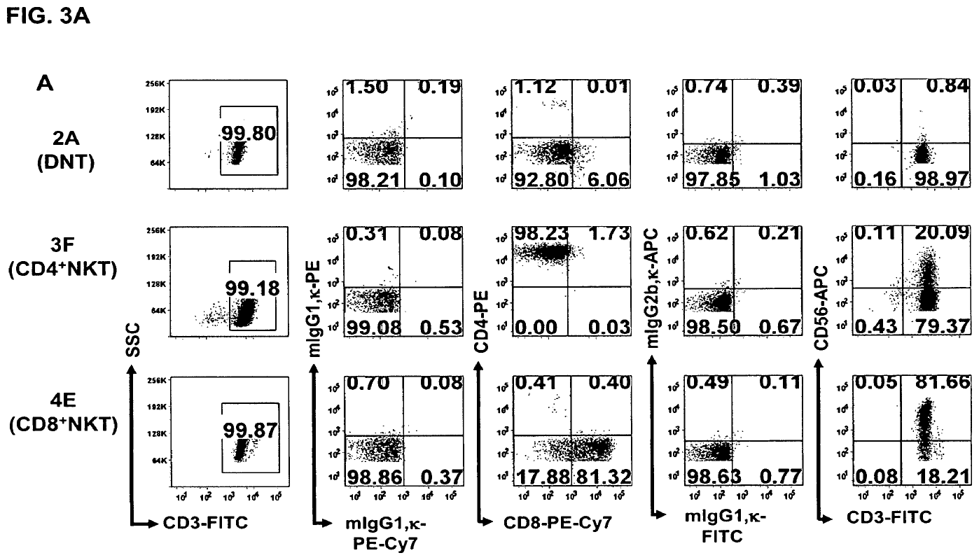

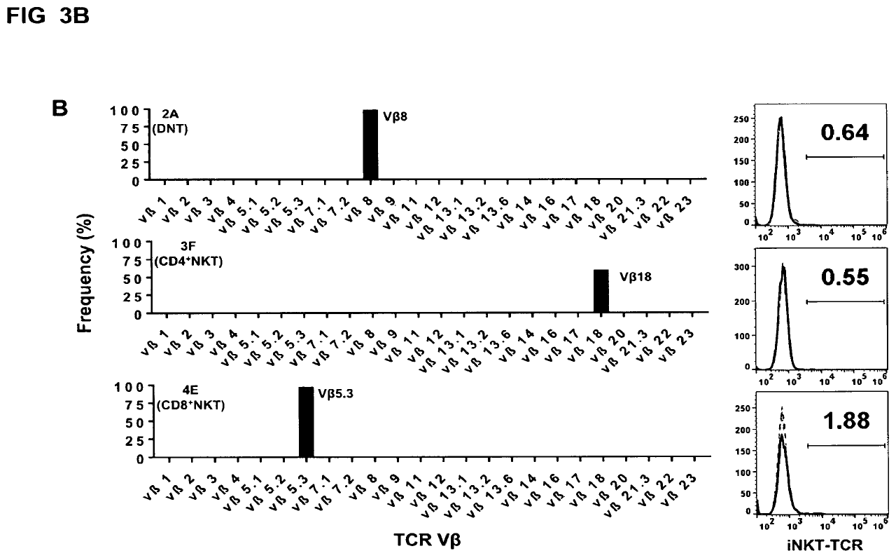

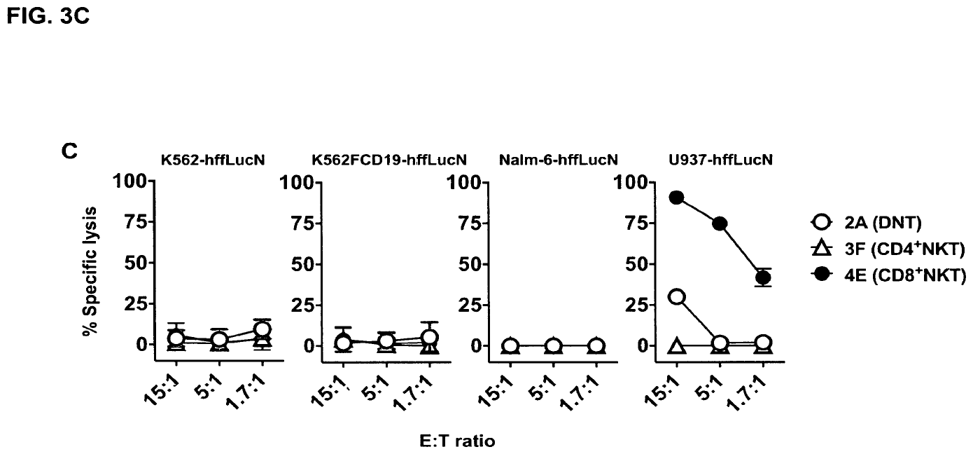

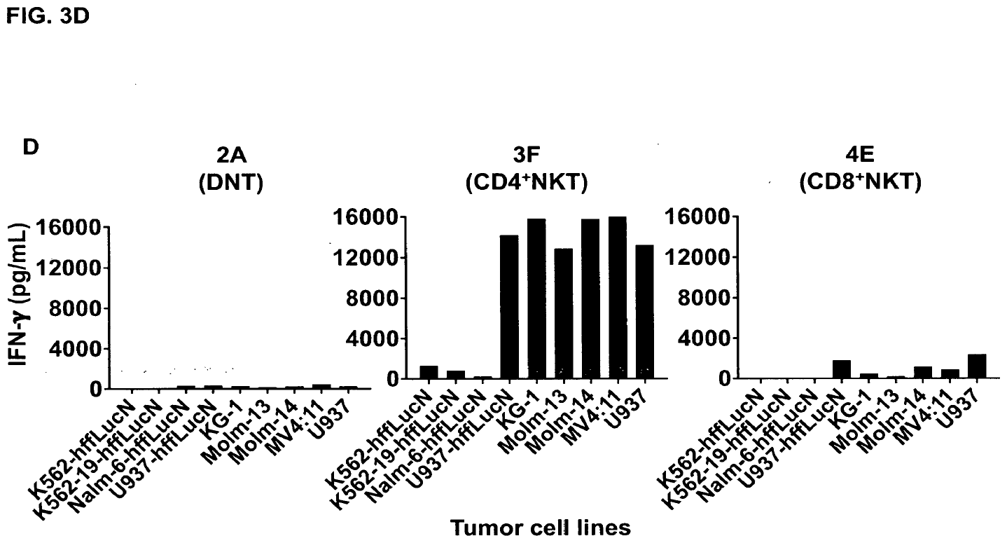

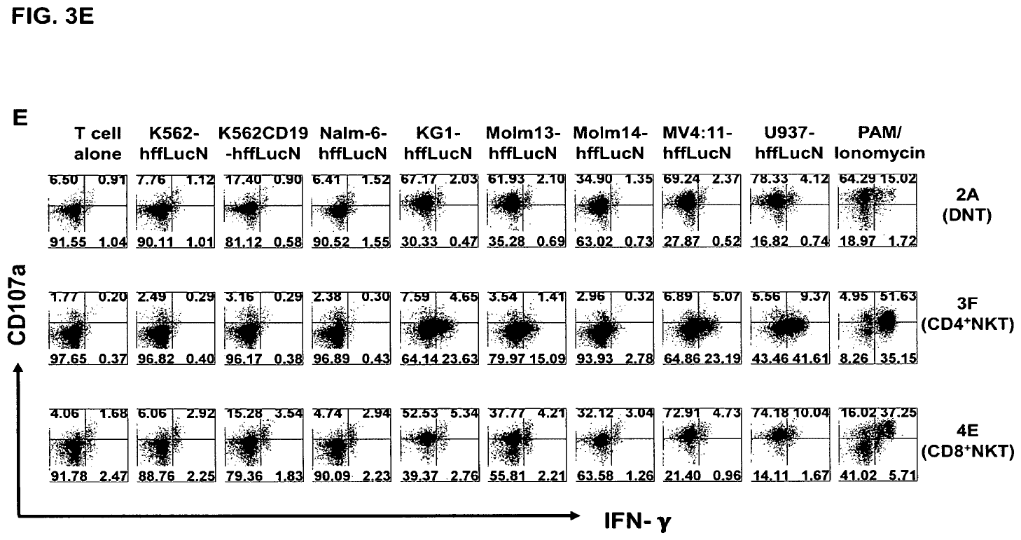

Clones from DNT cultures were generated to evaluate single DNT and NKT cell clones for anti-AML activity. Approximately 30 clones were derived from 3, 3, and 16 plates of 30, 3, and 0.3 cell per well in 96-well plates from DNT cultures by a limiting dilution, respectively, and 10 clones were expandable. As shown in FIG. 3A, flow cytometric analysis demonstrated that the phenotypes of 2A, 3F, and 4E clones were CD3⁺CD4⁻CD8⁻CD56⁻, CD3⁺CD4⁺CD8⁻CD56⁺, and CD3⁺CD4⁻CD8⁺CD56⁺, respectively, and were considered DNT, CD4⁺ NKT, and CD8⁺ NKT cells, respectively. Furthermore, FIG. 3B shows that 2A, 3F, and 4E clones were phenotypically positive for Vβ8, Vβ18, and Vβ5.3, and negative for iNKT TCR (Vα24-Jα18).

FIG. 3C shows that the 4E (CD8⁺ NKT) clone surprisingly killed U937 AML cells more potently than the 2A (DNT) clone in all three effector/target (E/T) ratios in a 4-h luciferase-based cytotoxicity assay. The 3F (CD4⁺ NKT) clone did not kill AML. None of the clones killed CD19⁺ Nalm-6 (B-ALL), CD19 transfected K562 (erythroleukemia), or K562 leukemia cells.

FIG. 3D shows that the 3F (CD4⁺ NKT) clone remarkably released approximately 37 times and 15 times more IFN-γ than the 4E (CD8⁺ NKT) and 2A (DNT) clones in response to AML cell lines, including U937-hfflucN, KG-1, Molm-13, Molm-14, MV4:11, and U937. In addition, the 4E (CD8⁺ NKT) clone produced approximately 2.5-4 times more IFN-γ than the 2A (DNT) clone in response to AML cell lines (except for Molm-13).

Flow cytometry-based CD107α-IFN-γ assays were used to assess cellular cytotoxicity and intracellular IFN-γ levels in response to tumor cell stimulation. FIG. 3E shows that the 4E (CD8⁺ NKT) and 2A (DNT) clones were equally cytotoxic in terms of CD107a expression on all luciferase-expressing AML cells tested (KG-1, Molm-13, Molm-14, MV4:11, and U937), whereas the 3F (CD4⁺ NKT) clone produced more IFN-γ than the 4E (CD8⁺ NKT) and 2A (DNT) clones. Additional AML cell lines (HL60, Molm-13, Molm-14 and MV4:11) expressing luciferase were also generated and tested.

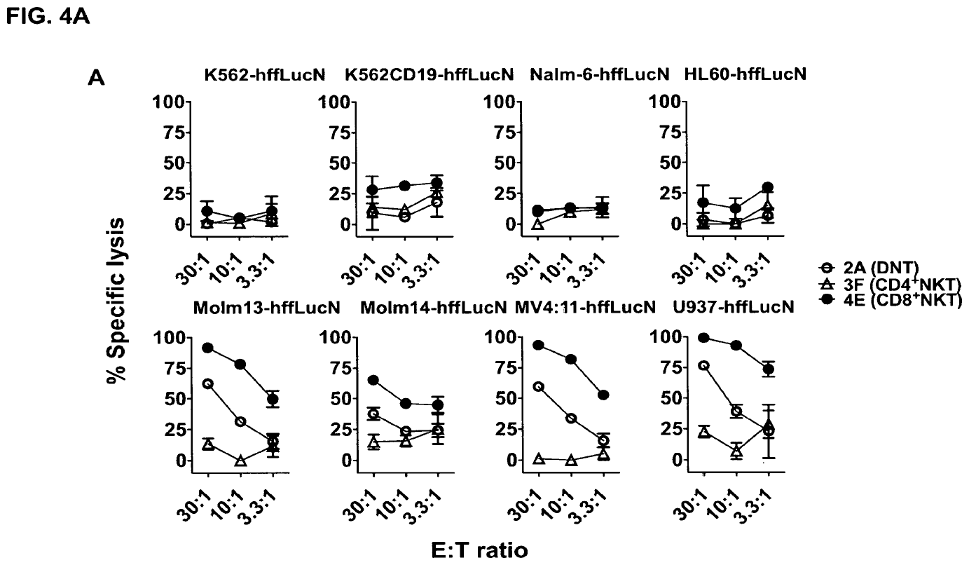

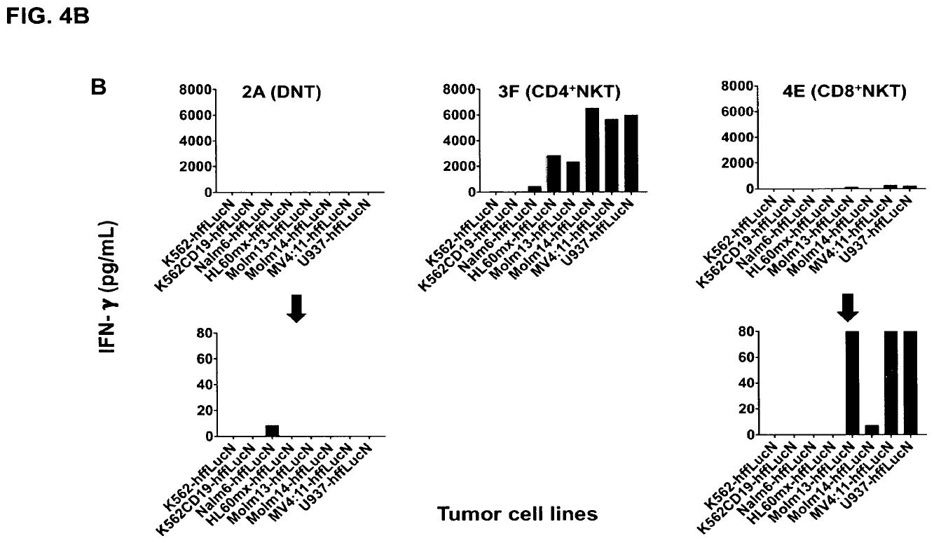

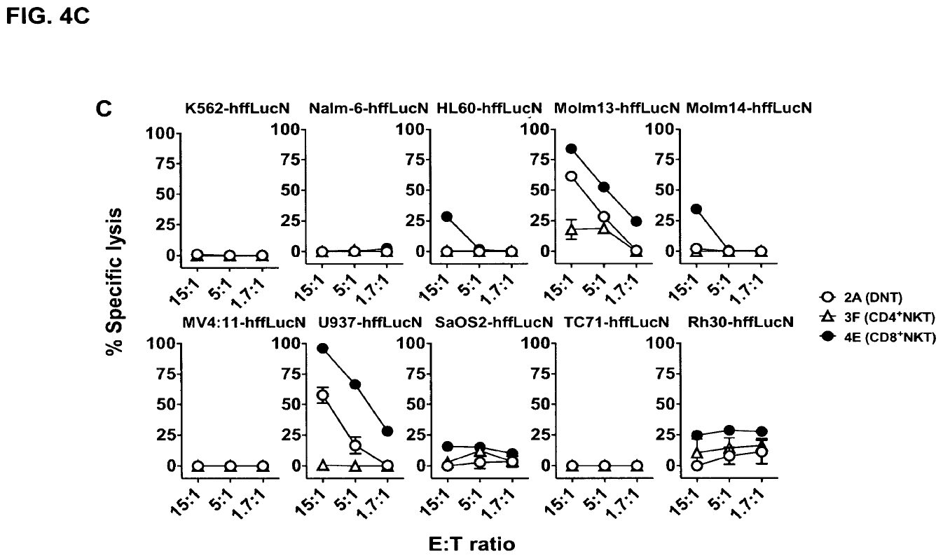

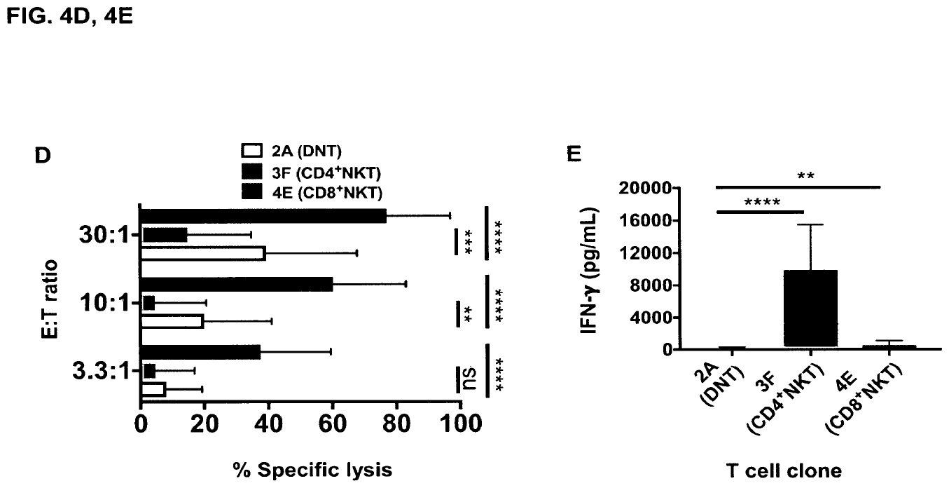

FIG. 4A and FIG. 4C show that 4E (CD8⁺ NKT) cells were more cytotoxic to HL60, Molm-13, Molm-14, MV4:11, and U937 cells expressing luciferase than 2A (DNT). FIG. 4B shows that 3F (CD4⁺ NKT) cells produced more IFN-γ in response to HL60, Molm-13, Molm-14, MV4:11, and U937 cells expressing luciferase than 2A (DNT). 4E (CD8⁺ NKT) cells also produced more INF-γ than 2A (DNT) in response to the majority of AML cell lines. As shown in FIG. 4C, CD8⁺ NKT cells did not kill certain luciferase-expressing sarcoma lines (Rh30, SaOS2, and TC71).

FIG. 4D and FIG. 4E show our statistical conclusion based on 5 and 2 independent assays, respectively that 4E (CD8⁺ NKT) are more potent than 2A (DNT) clone in killing AML cells at all E/T ratios tested whereas 3F (CD4⁺ NKT) clone produced almost 10000 fold more IFN-γ than 2A (DNT) clones in response to AML cells (p<0.0001). 4E (CD8⁺ NKT) cells also produced higher levels of IFN-γ than 2A (DNT) clones (p<0.01). These results suggest that both CD4⁺ NKT and CD8⁺ NKT cells are potent anti-AML effector cells.

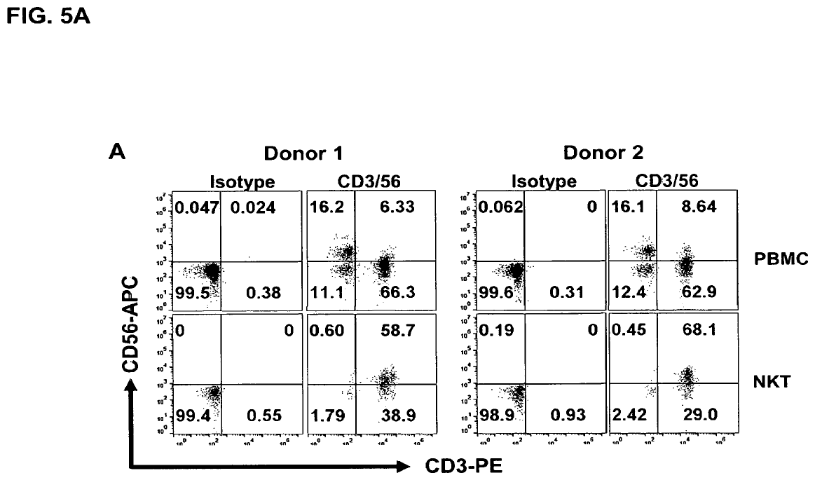

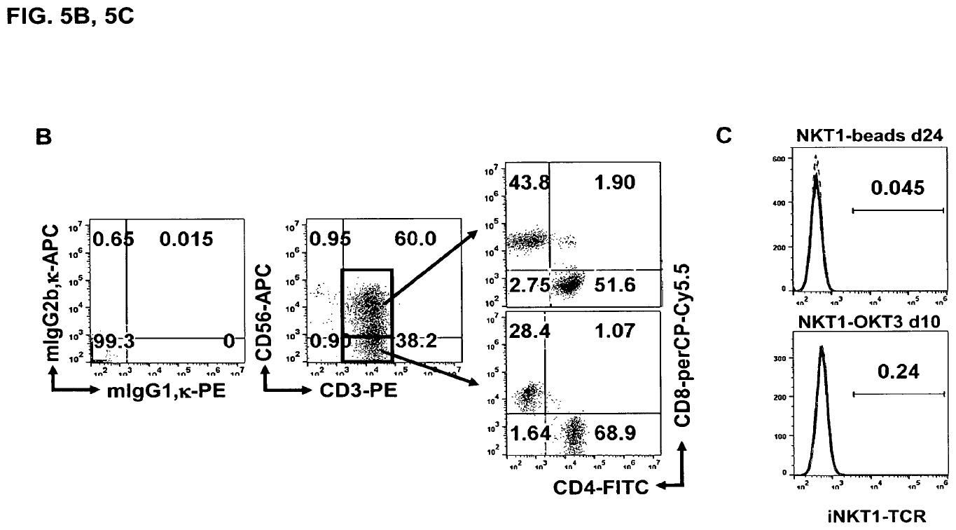

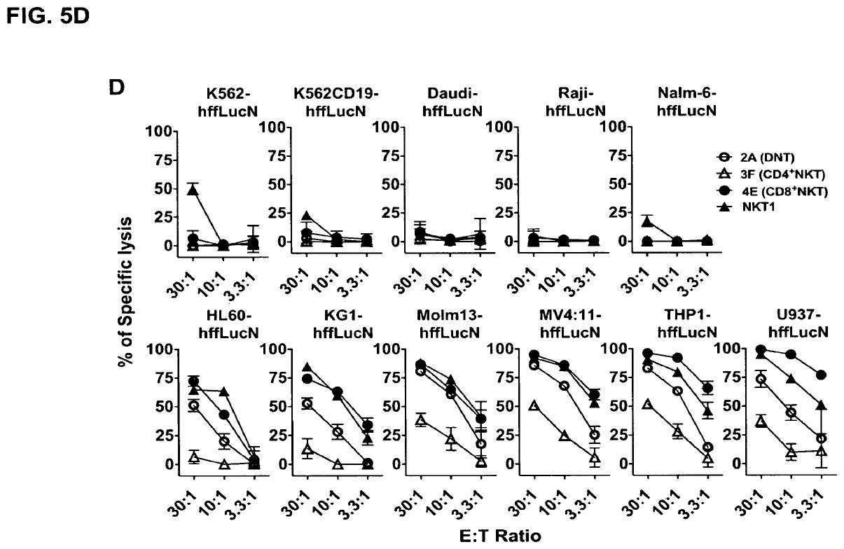

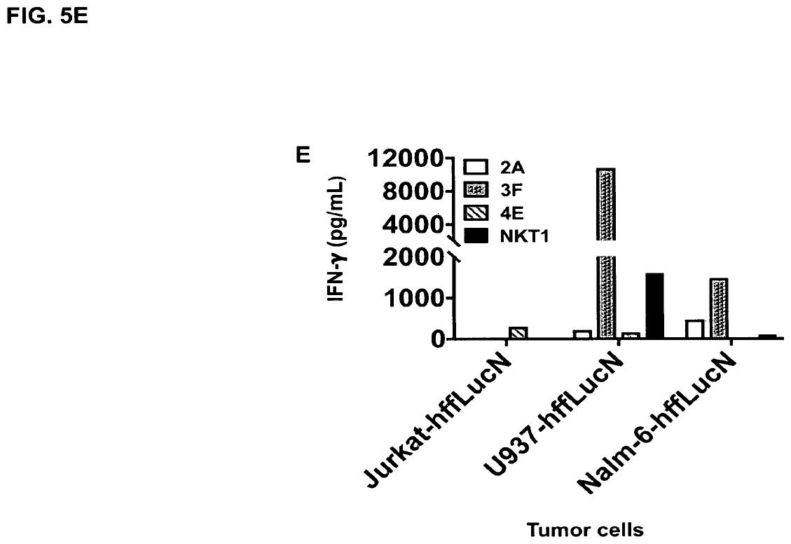

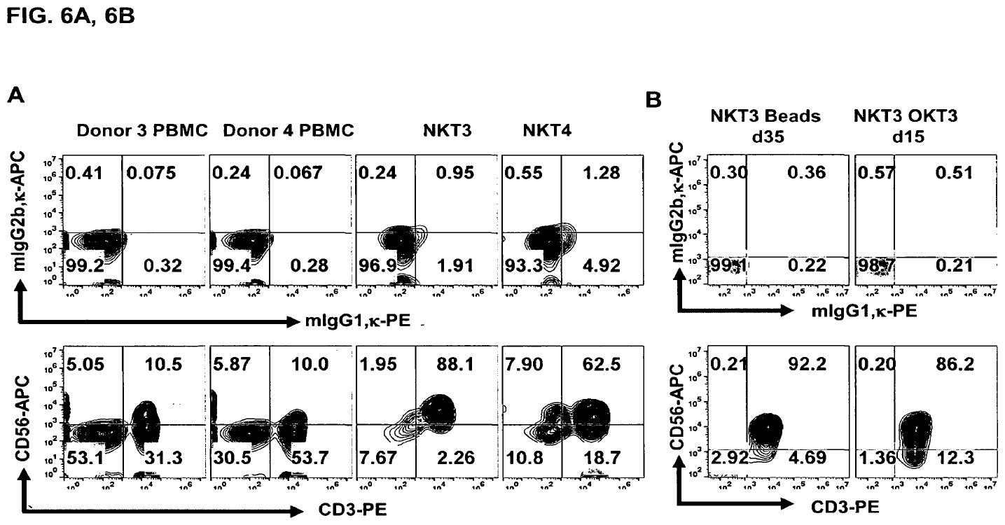

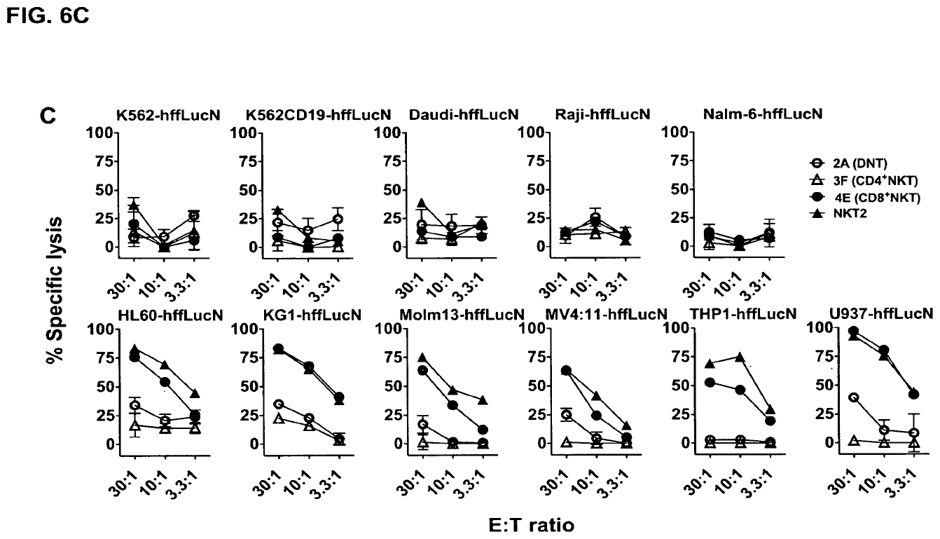

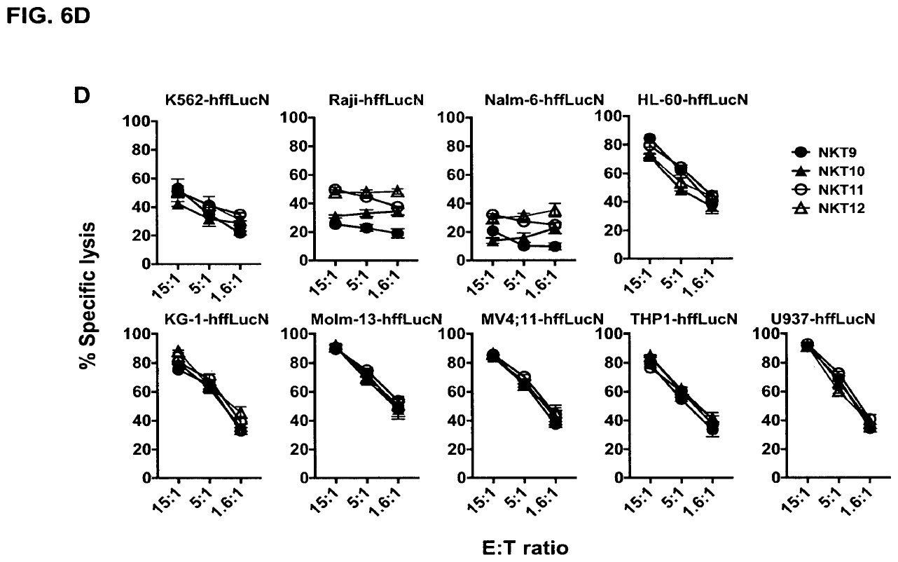

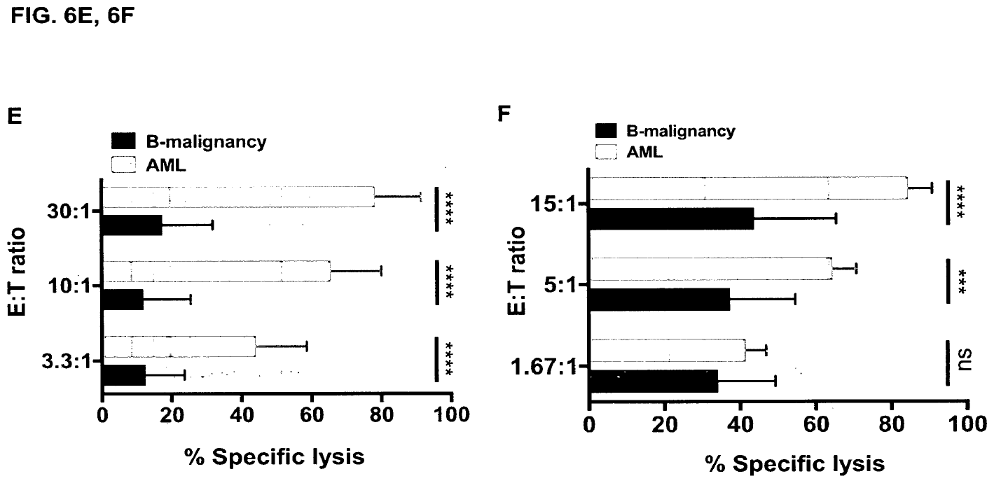

To evaluate whether polyclonal NKT cells can functionally imitate the 4E (CD8⁺ NKT) clone, CD3⁺CD56⁺ NKT cells were initially isolated from four healthy donors using a CD3⁺CD56⁺ NKT Cell Isolation Kit. Isolated CD3⁺CD56⁺ NKT cells were then activated in a 24-well plate with CD3/CD28 microbeads for 3 days, cultured with IL-2 upon removal of beads, and expanded in T25 flasks with OKT3, allogeneic PBMCs, EBV-LCL (Epstein-Barr virus-transformed lymphoblastoid cell lines), and IL-2. FIG. 5A and FIG. 6A show that CD3⁺CD56⁺ NKT cells comprised 6-10% of PBMCs and were enriched to 58-88% after isolation. FIG. 5B and FIG. 6B show that upon CD3/CD28 bead activation and subsequent expansion with anti-CD3 antibody (OKT3) and IL-2, 60-82% of cells in two cultures remained CD3⁺CD56⁺. FIG. 5C shows that iNKT TCR staining of NKT1 cells from two cultures was negative, suggesting no contamination of iNKT cells in CD3⁺CD56⁺ NKT cells. FIG. 5D and FIG. 6C show that activated and cultured CD3⁺CD56⁺ NKT1 and NKT2 bulks derived from donors 1 and 2 killed all AML cells lines as potently as the 4E (CD8⁺ NKT) clone in an E/T ratio dependent manner. FIG. 5E shows that NKT1 cells specifically produced IFN-γ in response to U937 AML cells. FIG. 6D shows that four additional CD3⁺CD56⁺ NKT cells (NKT9, NKT10, NKT11, NKT12) killed all AML cell lines despite a little background of killing on K562 CML, Raji B-NHL and Nalm-6 B-ALL cells in this experiment. As shown in FIG. 6E and FIG. 6F, CD3⁺CD56⁺ NKT cells manufactured under this condition significantly killed AML cells compared to B-cell malignancies (p<0.001 excluding E/T of 1.67:1).

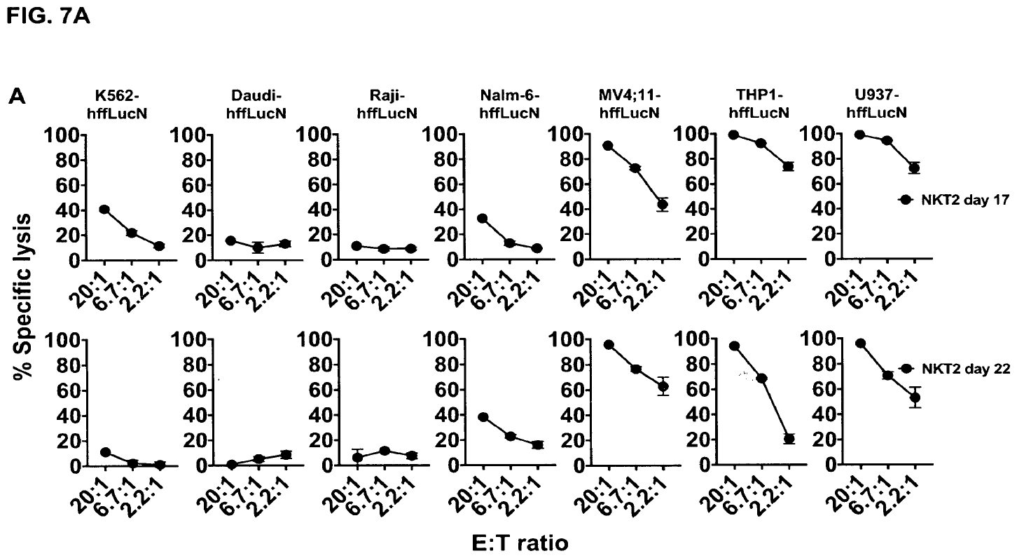

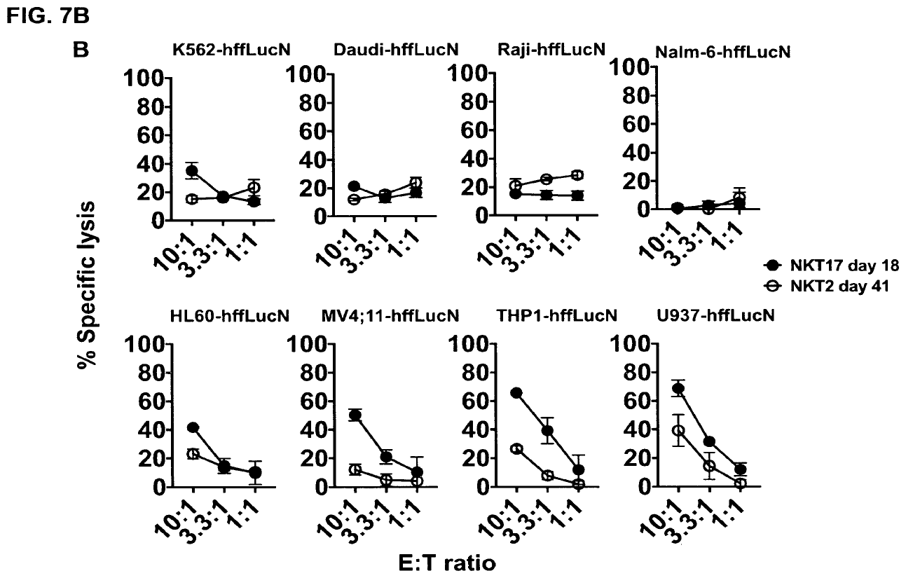

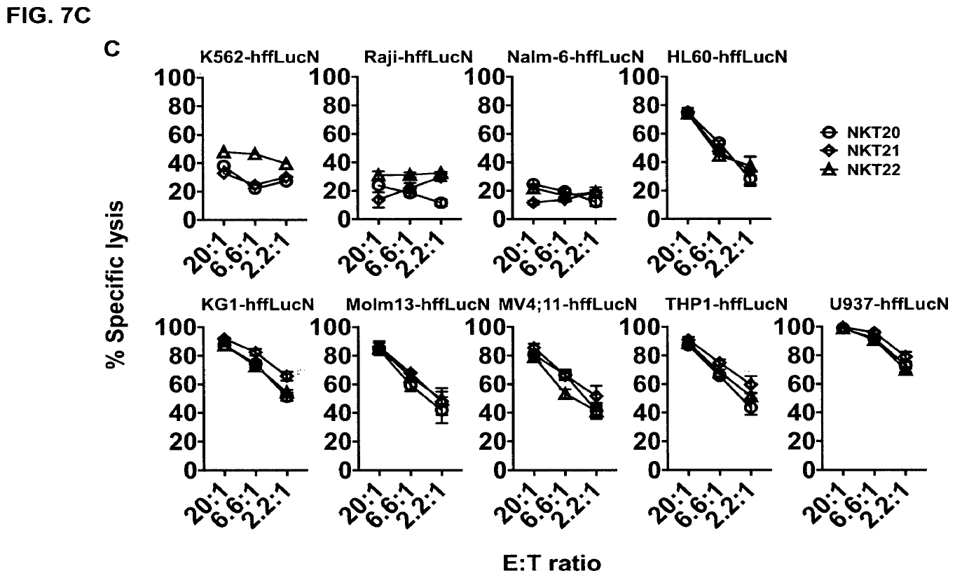

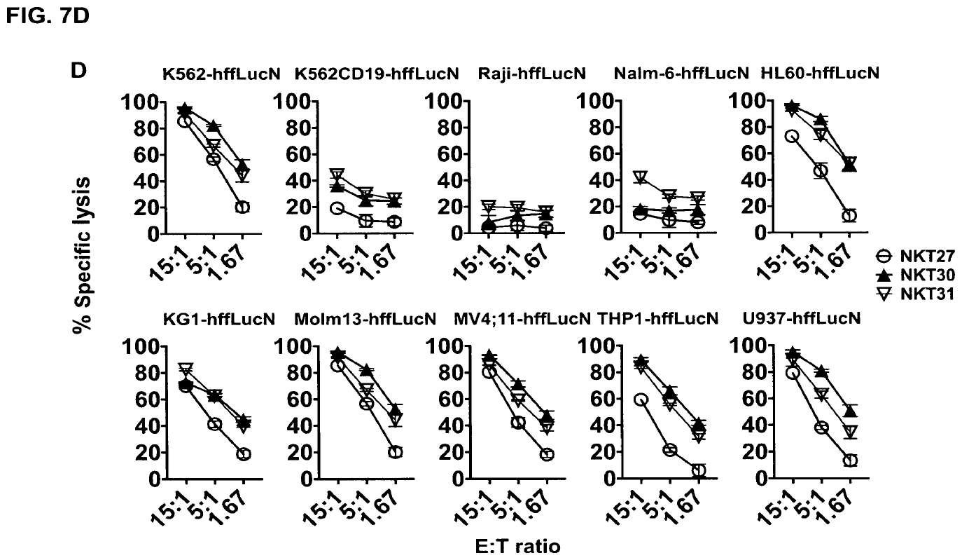

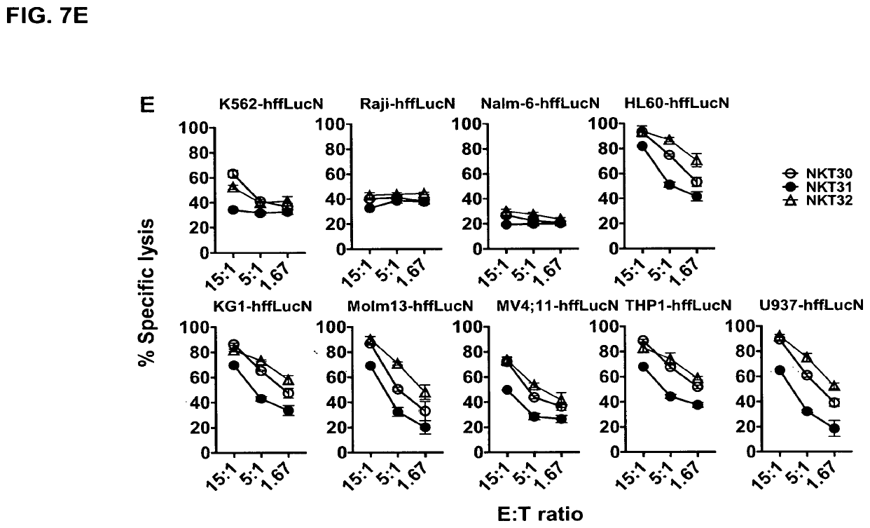

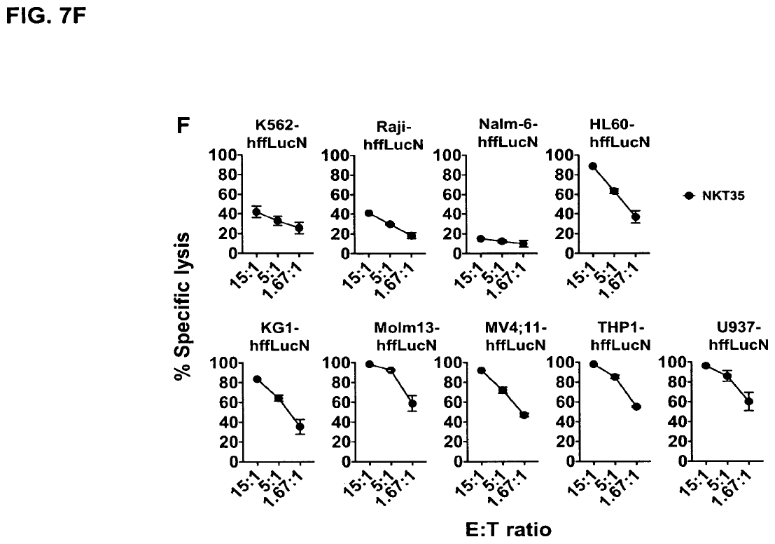

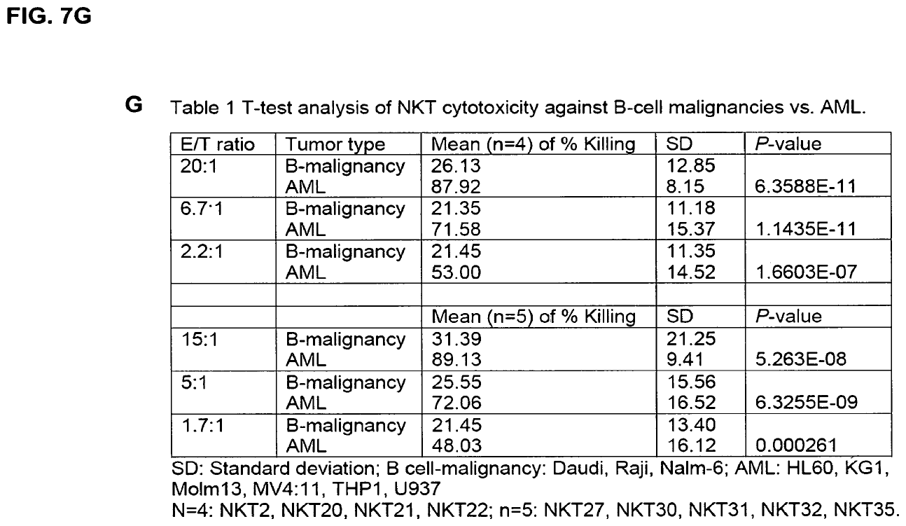

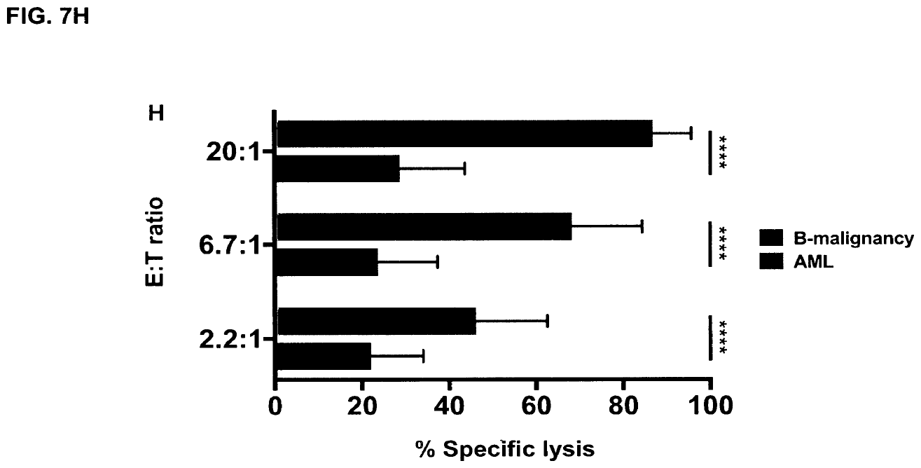

To demonstrate that CD3⁺CD56⁺ NKT cells isolated from healthy blood donors reproducibly kill AML cell lines, we used OKT3-coated flasks or plates and IL-2 to activate CD3⁺CD56⁺ NKT cells for 3 days and cultured them in medium with IL-2 for additional four days, and restimulated them with soluble OKT3 for 3 days. FIG. 7A and FIG. 7B shows that NKT2 on day 17, 22 and 41 after culture displayed potent cytotoxicity against luciferase-expressing AML cells (HL60, MV4:11, THP-1 and U937) whereas no or low background of killing on K562 CML, Daudi B-NHL, Raji B-NHL and Nalm-6 B-ALL cell lines by NKT2 was observed.

With this culture condition, 9 more CD3⁺CD56⁺ NKT cells were isolated and expanded in culture with OKT3 and IL-2. FIG. 7C, FIG. 7D, FIG. 7E and FIG. 7F show that all 9 NKT cells (NKT17, NKT20, NKT21, NKT22, NKT27, NKT30, NKT31, NKT32, NKT35) killed all luciferase-expressing AML cell lines tested (HL60, KG1, Molm-13, MV4:11, THP-1 and U937) in an E/T ratio dependent manner while they showed no or low levels of killing on Daudi B-NHL, Raji B-NHL and Nalm-6 B-ALL as well as variable levels of killing on K562 CML cells. FIG. 7G and FIG. 7H show statistical conclusion that CD3⁺CD56⁺ NKT cells from 9 healthy donors using an alternative method of manufacturing preferentially killed all 6 AML cell lines versus B-cell malignancies at all E/T ratios (p<0.0001).

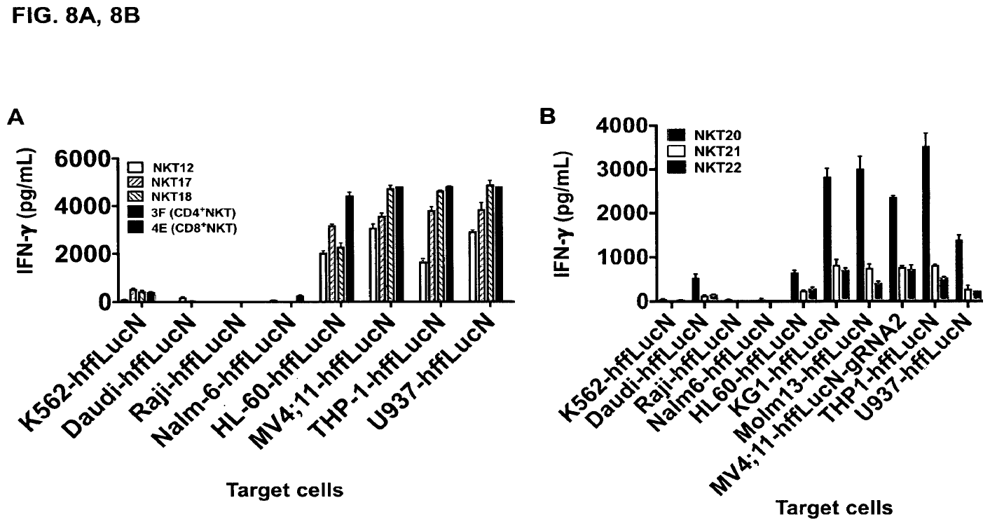

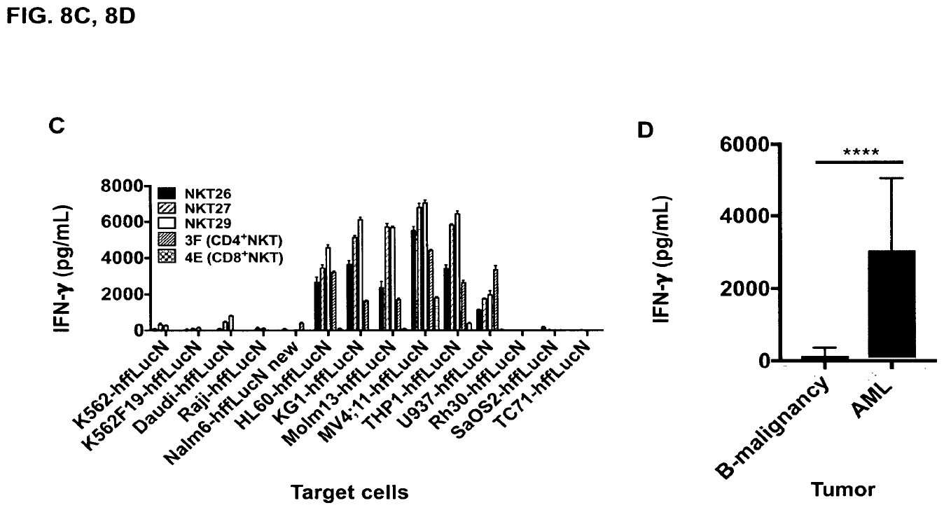

As shown in FIG. 8A, FIG. 8B, FIG. 8C and FIG. 8D, those CD3⁺CD56⁺ NKT cells (n=9) also produced approximately 3000 fold higher levels of IFN-γ cytokine in co-culture with all luciferase-expressing AML cell lines (mean±SD, 3053.6±1988.8) compared to co-culture with luciferase-expressing B-cell malignancies including Daudi, Raji and Nalm-6 cells (mean±SD, 138.94±211.62) (p<0.0001). CD3⁺CD56⁺ NKT cells did not recognize luciferase-expressing sarcoma cell lines (Rh30, SaOS2 and TC71). In addition, IFN-γ levels produced by NKT cells in response to AML cell stimulation were comparable to control 3F (CD4⁺ NKT) clone.

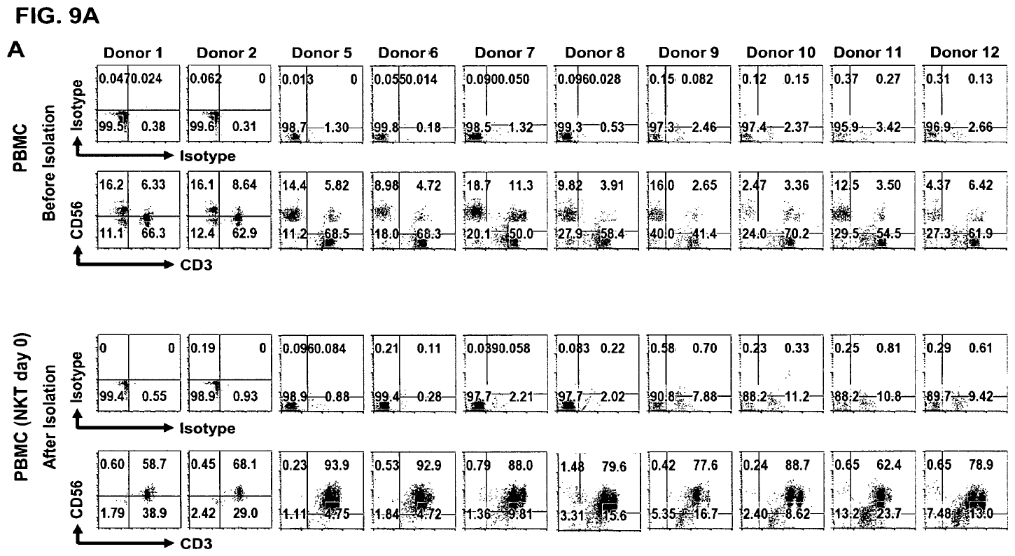

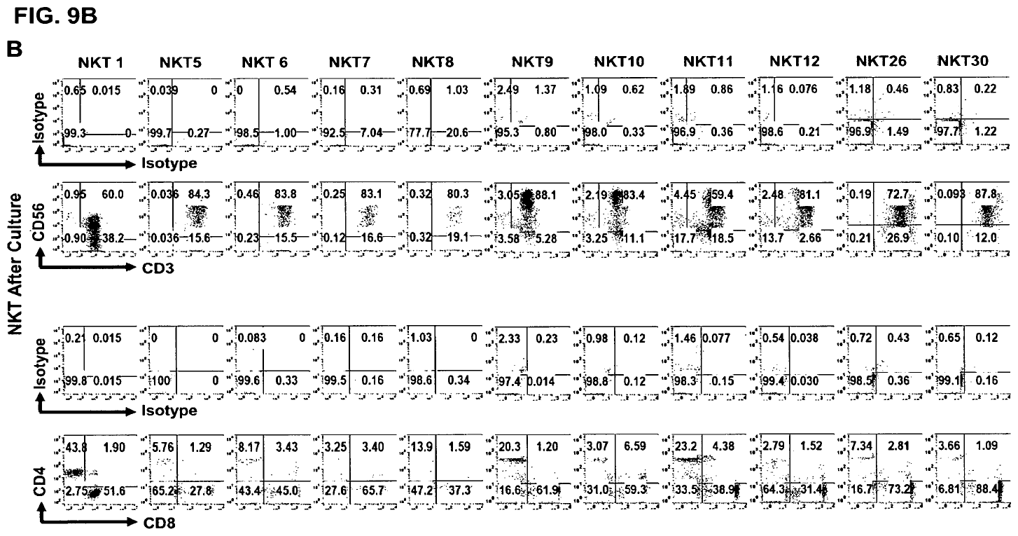

FIG. 9A shows the percentages of CD3⁺CD56⁺ NKT in PBMCs from individual 10 healthy donors ranging from 2.65 to 11.3% (mean±SD, 5.67±2.68) and their enrichment purity ranging from 58.7 to 93.9% (mean±SD, 79.72±12.14) after microbead selection. FIG. 9B shows that CD3⁺CD56⁺ NKT cell percentages after culture for 2-4 weeks remained high (mean±SD, 78.54±9.706, n=11), indicating that the culture conditions minimally altered the percentages of CD3⁺CD56⁺ NKT cells. In addition, as shown in FIG. 9B, CD3⁺CD56⁺ NKT cell populations after culture were composed of CD4⁺, CD8⁺ and CD4⁻CD8⁻ cells.

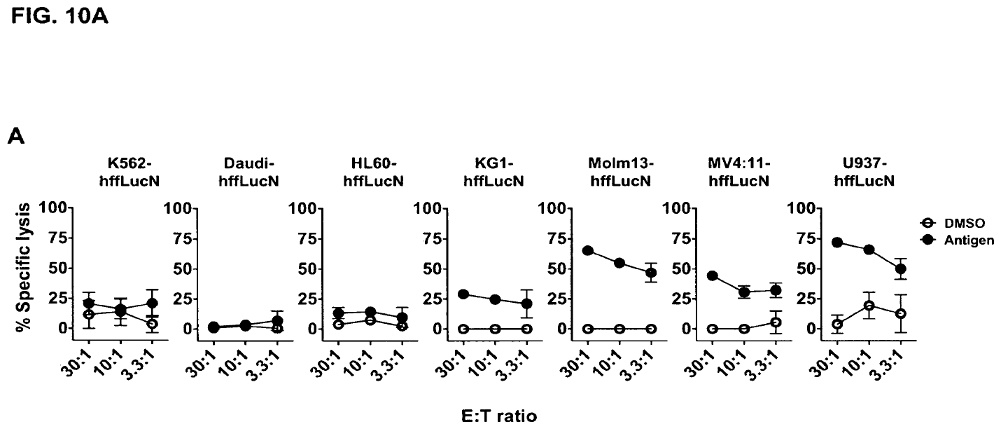

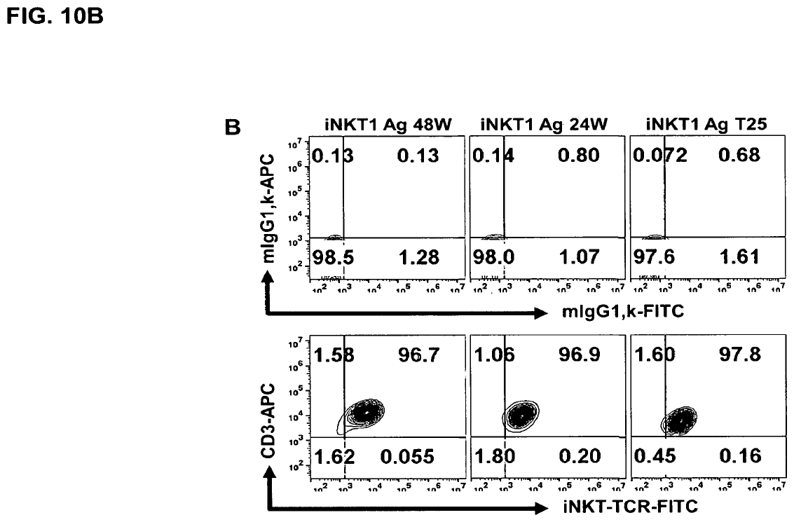

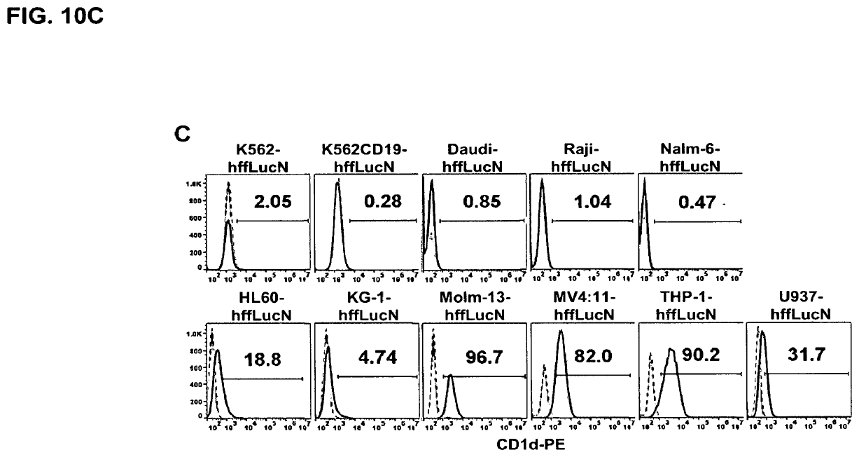

To evaluate whether iNKT cells also kill AML cells and their cytotoxicity can be enhanced by α-GalCer and correlated with CD1d expression, iNKT cells from one healthy donor were isolated, activated, and expanded. FIG. 10A shows that iNKT1 cells from donor 1 did not kill AML cell lines and B-cell leukemia and lymphoma lines in the absence of α-GalCer antigen. In contrast to innate anti-AML activity of CD3⁺CD56⁺ NKT cells, iNKT-mediated cytotoxicity against AML was only demonstrated in the presence of α-GalCer. FIG. 10B shows that iNKT cells in all three cultures expressed cell surface markers CD3⁺ and iTCR⁺. FIG. 10C shows that all tested AML cells lines were CD1d⁺ and that CD1d expression was generally correlated with cytotoxicity, except for HL60 and U937. These results suggest that CD3⁺CD56⁺ NKT cells may recognize AML cells in a CD1d-restricted manner.

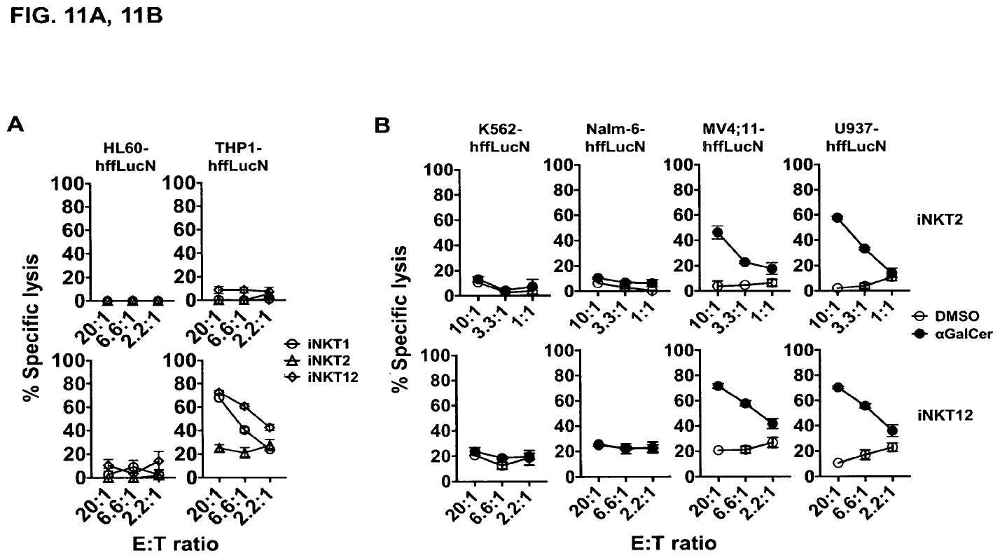

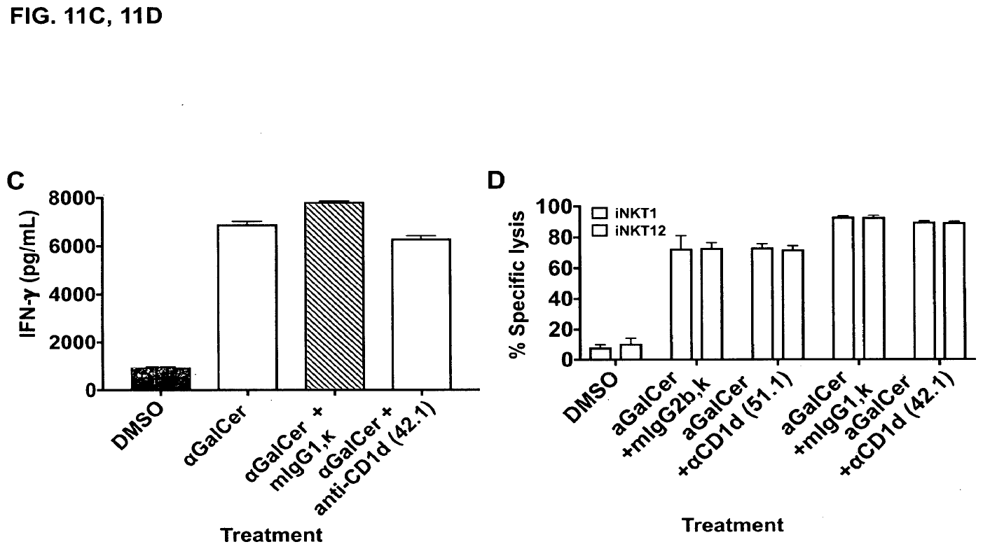

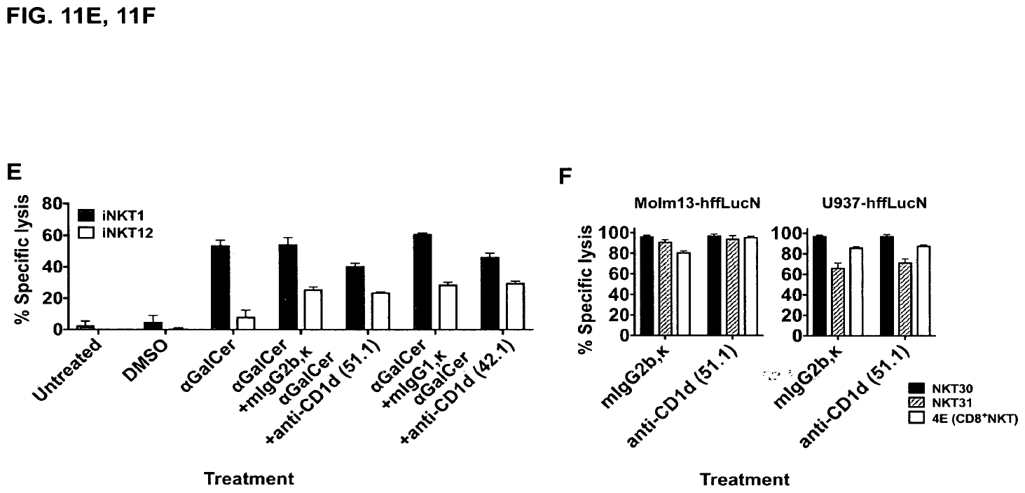

To further demonstrate that iNKT cells can not kill AML cells in the absence of α-GalCer antigen, two more iNKT cells (iNKT2 and iNKT12) were generated from healthy donors. FIG. 11A and FIG. 11B shows that iNKT2 and iNKT12 as well as iNKT1 cells killed CD1d⁺ AML cells (HL60, MV4:11, THP-1 and U937) pulsed with α-GalCer antigen but not DMSO. CD1d⁻ K562 CML and Nalm-6 B-ALL cells pulsed with α-GalCer antigen were minimally killed by all three iNKT cells, suggesting that unlike CD3⁺CD56⁺ NKT cells, iNKT cells do not have an innate cytotoxicity against AML cells.

Anti-human CD1d monoclonal antibodies (clone 42.1 from BD Biosciences and clone 51.1 from BioLegend) have been used in blocking CD1d-restricted presentation of α-GalCer antigen to iNKT cells at a concentration of 10 μg/mL (Ishihara et al. J Immunol. 2000; 165(3):1659-64; Mangan et al. J Immunol. 2013; 191(1):30-34; Jahnke et al. Front Immunol. 2019; 10:1542). FIG. 11C, FIG. 11D and FIG. 11E shows that anti-human CD1d antibodies from both clone 42.1 and 51.1 at 50 μg/mL (FIG. 11C), 10 μg/mL (FIG. 11D) and 30 μg/mL (FIG. 11E) were unable to significantly block CD1d-mediated antigen presentation to iNKT cells in both ELISA and luciferase-based killing assays. Moreover, an anti-human CD1d antibody (clone 51.1) at 30 μg/mL also failed in blocking NKT30-, NKT-31- and 4E (CD8⁺ NKT-mediated killing on Molm-13 and U937 AML cells (FIG. 11F).

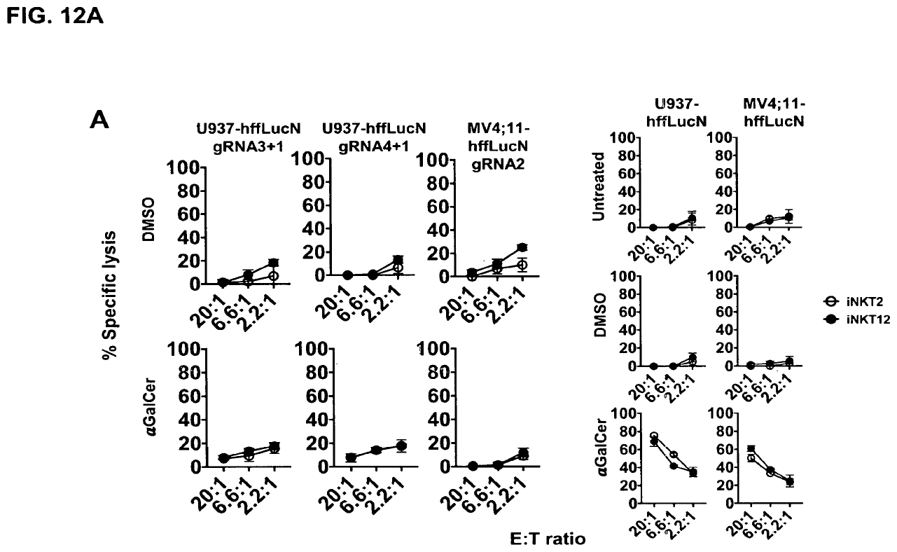

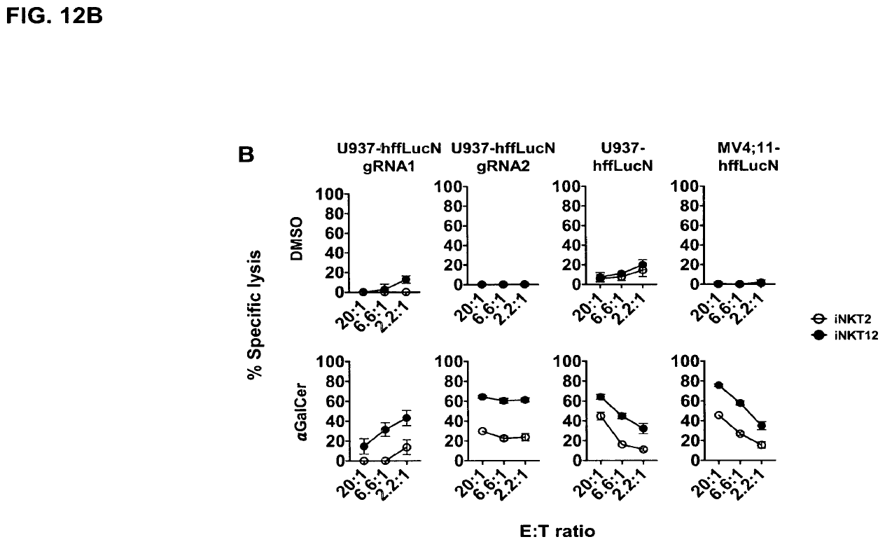

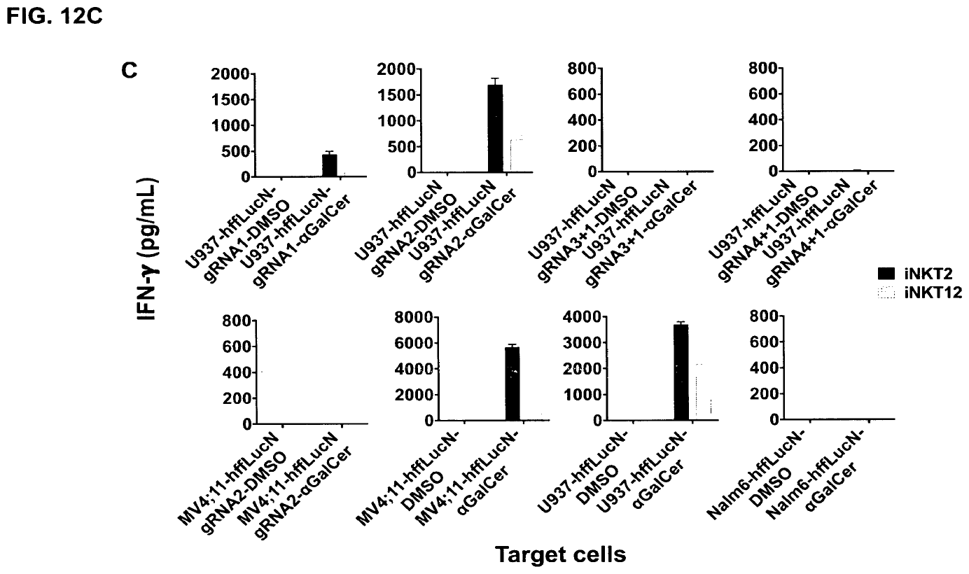

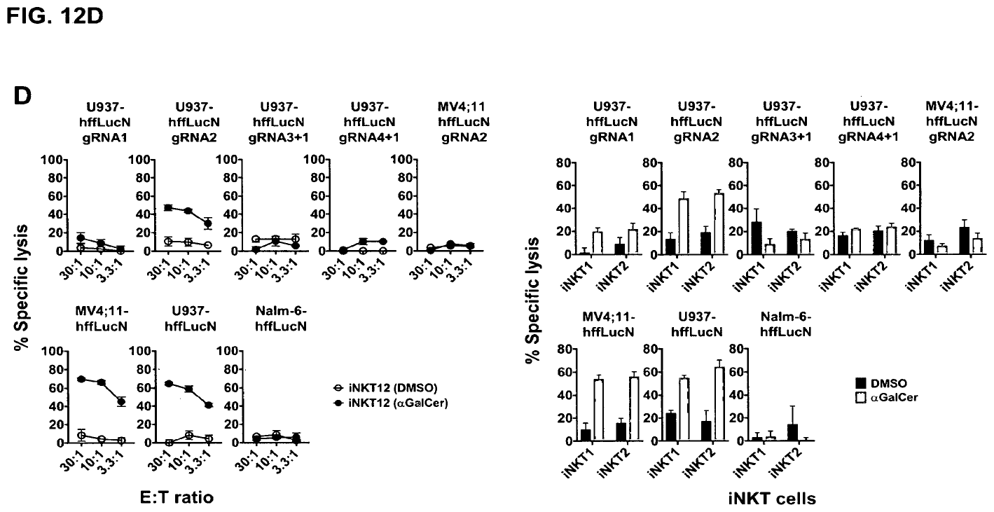

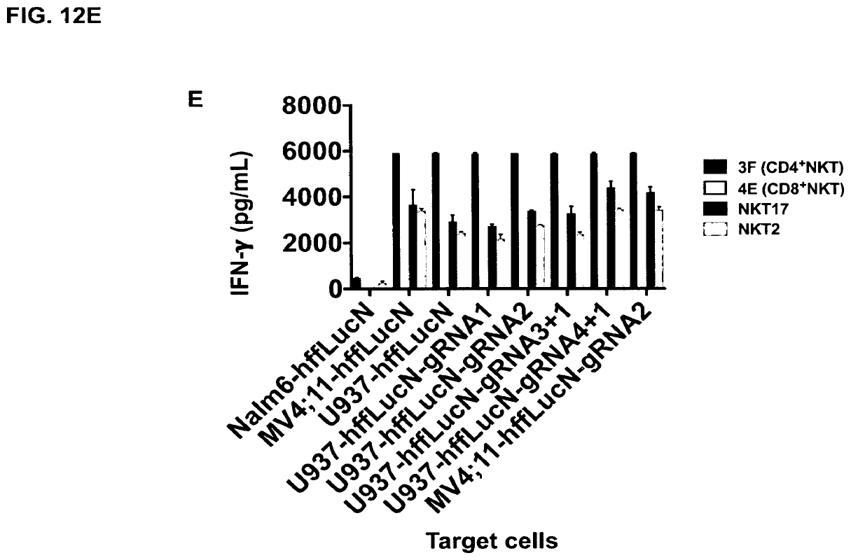

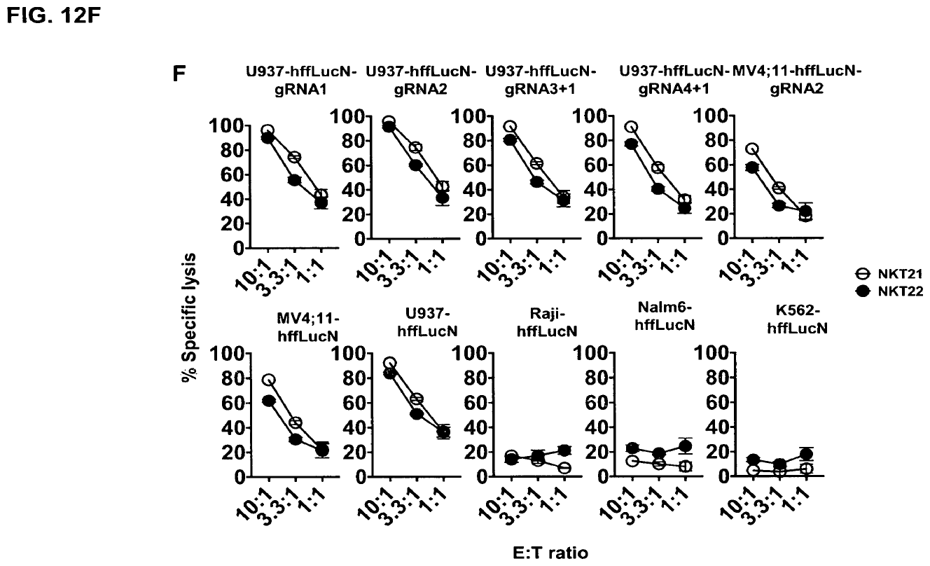

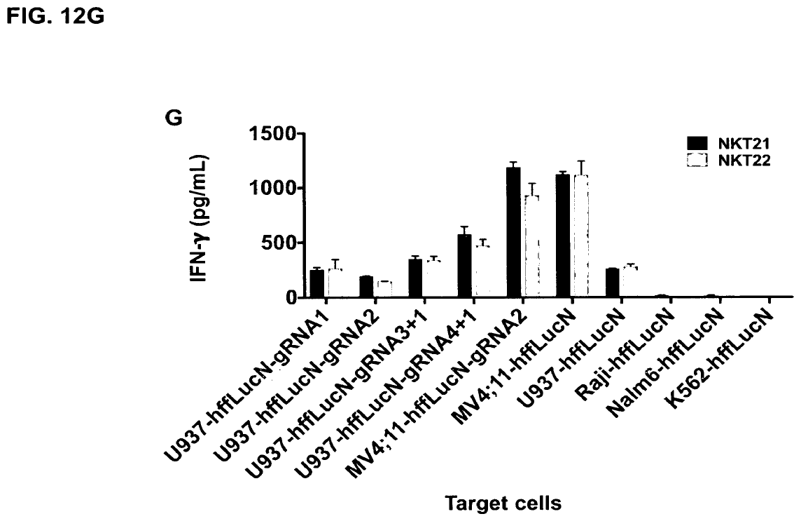

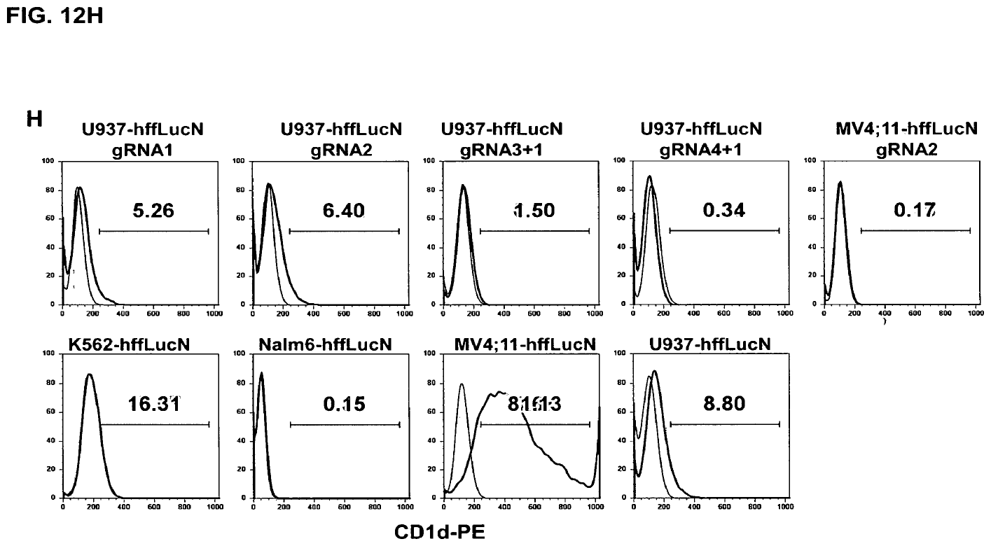

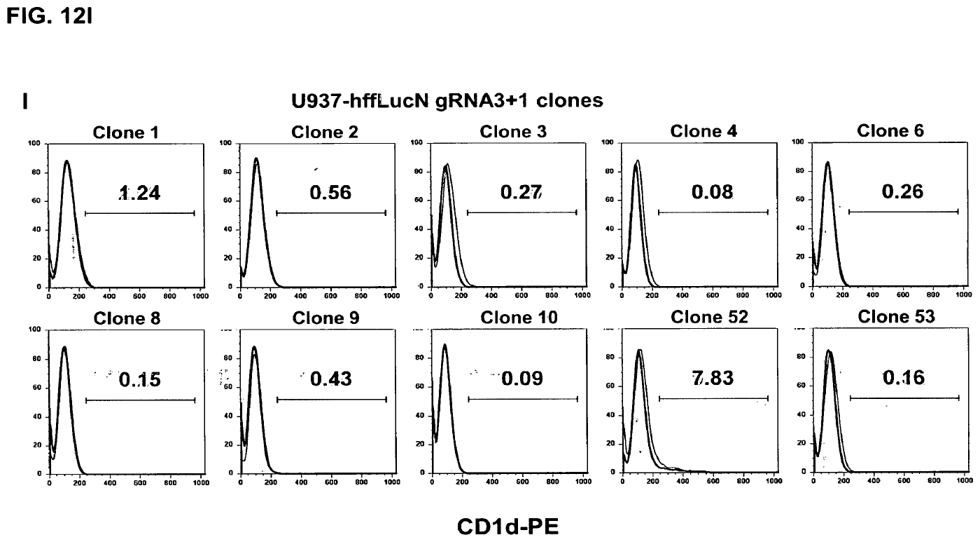

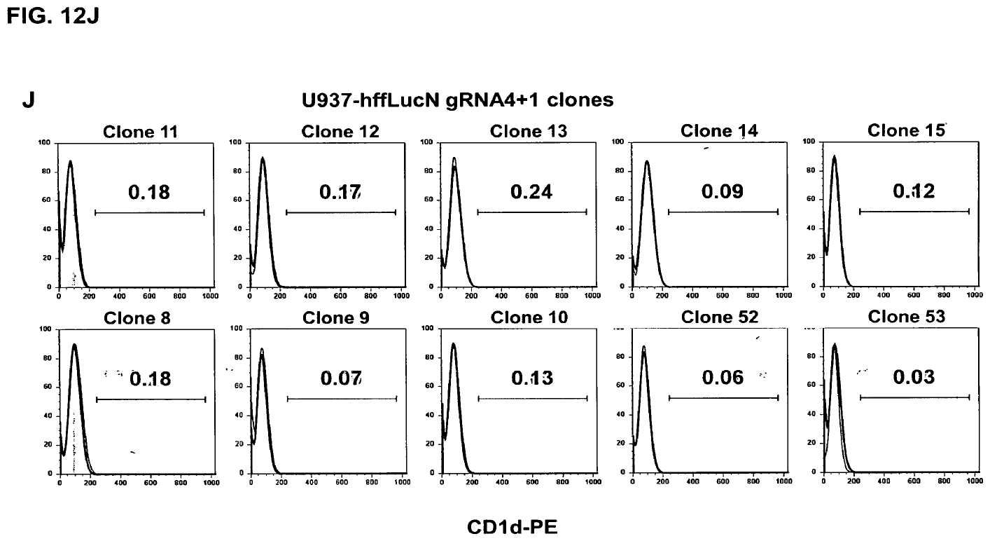

To further dissect whether CD3⁺CD56⁺ NKT cell-mediated recognition of AML cells depends on CD1d molecule on AML cells, CRISPR-Cas9 genome editing technology was applied to knock out CD1d in luciferase-expressing U937 and MV4:11 AML cells. FIG. 12A, FIG. 12B, FIG. 12C and FIG. 12D show that lentiviral transduction of gRNA 3+1 or 4+1 in luciferase-expressing U937 or gRNA2 in luciferase-expressing MV4:11 AML cells abolished cytotoxicity and IFN-γ production by iNKT2 and iNKT12 as well as iNKT1 in recognition of the target cells pulsed with α-GalCer antigen. Control U937 and MV4:11 cells but not CD1d⁻ Nalm-6 cells pulsed with α-GalCer antigen were recognized by those three iNKT cells. However, lentiviral transduction of gRNA 1 or gRNA2 in luciferase-expressing U937 cells did not completely abolish iNKT cell cytotoxicity and IFN-γ production compared to wildtype U937 cells.

We also evaluated whether CD3⁺CD56⁺ NKT cells recognize CD1d knock out AML cells. FIG. 12E, FIG. 12F and FIG. 12G show that CD3⁺CD56⁺ NKT2, NKT17, NKT21 and NKT22 as well as 3F (CD4⁺ NKT) clone recognized CD1d knock out AML cells (U937-hffLucN-gRNA3+1, 4+1 or MV4:11-hffLucN-gRNA2) in both killing and IFN-γ ELISA assays.

FIG. 12H, FIG. 12I and FIG. 12J show that CD1d expression in CD1d gRNA3+1, 4+1 knock out U937 or gRNA2 knock out MV4:11 cells was completely negative on the cell surface compared to wildtype counterparts and CD1d⁻ K562 and Nalm-6 control cells. CD1d expression in gRNA1 or gRNA2 knock out U937 cells remained positive. All 20 single cell clones derived from gRNA3+1 or 4+1 U937 cells were negative for CD1d surface expression. The flow cytometric analysis of CD1d knockouts strongly correlates with iNKT cell functional assays. Our data suggest that CD3⁺CD56⁺ NKT-mediated innate recognition of AML cells may not require CD1d expression on target cells.

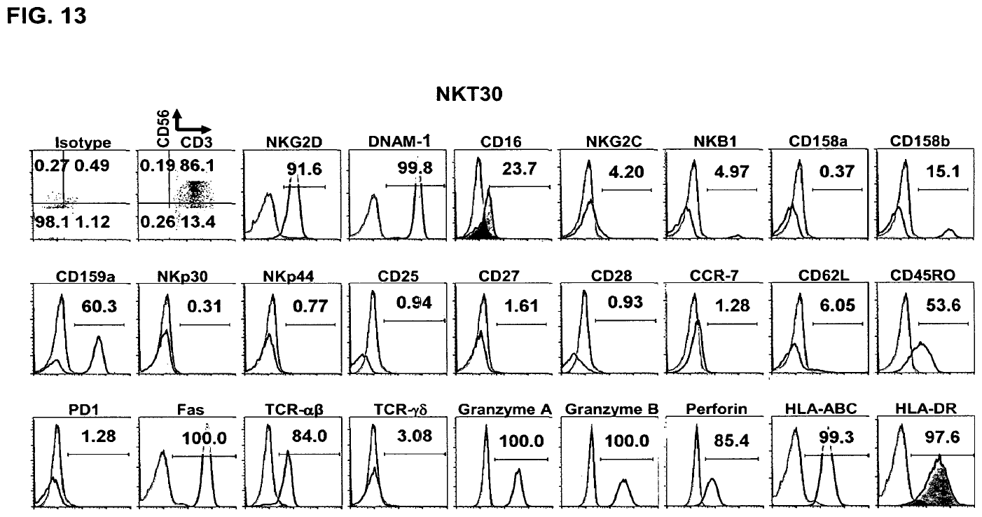

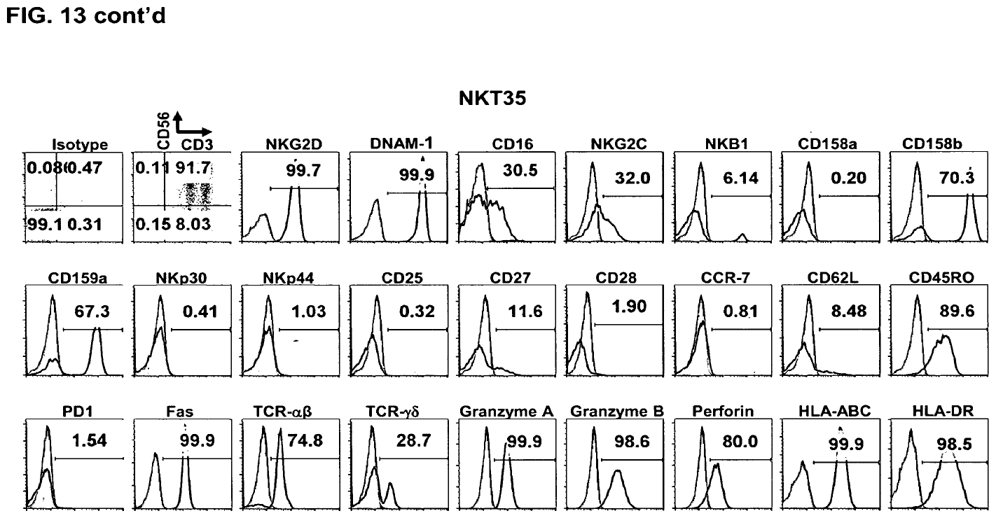

As shown in FIG. 13, the phenotypes of CD3⁺CD56⁺ NKT30 and NKT35 after culture. Both NKT cells expressed markers of NK cells and T cells such as CD56, NKG2D, DNAM-1, CD16, NKG2C, NKB1 (KIR3DL1), CD158b (KIR2DL2/L3), CD159a (NKG2A), CD3, CD45RO, FAS, TCRαβ, TCRγδ, granzyme A, granzyme B, perforin, HLA-ABC and HLA-DR.

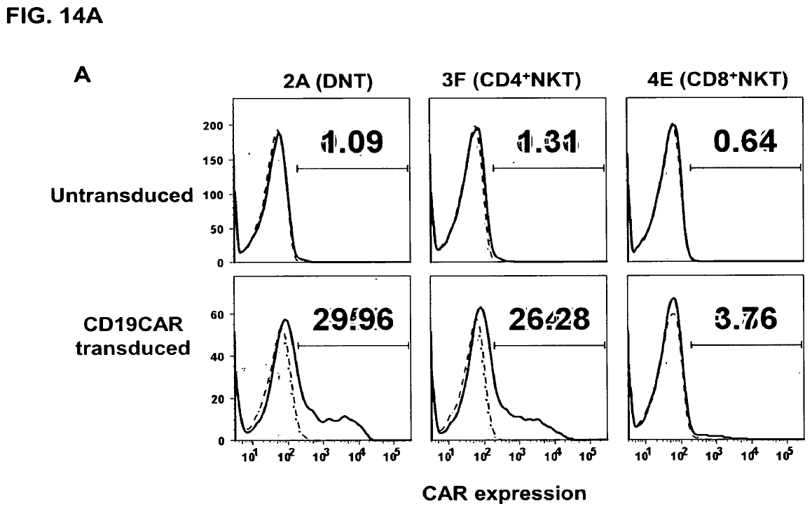

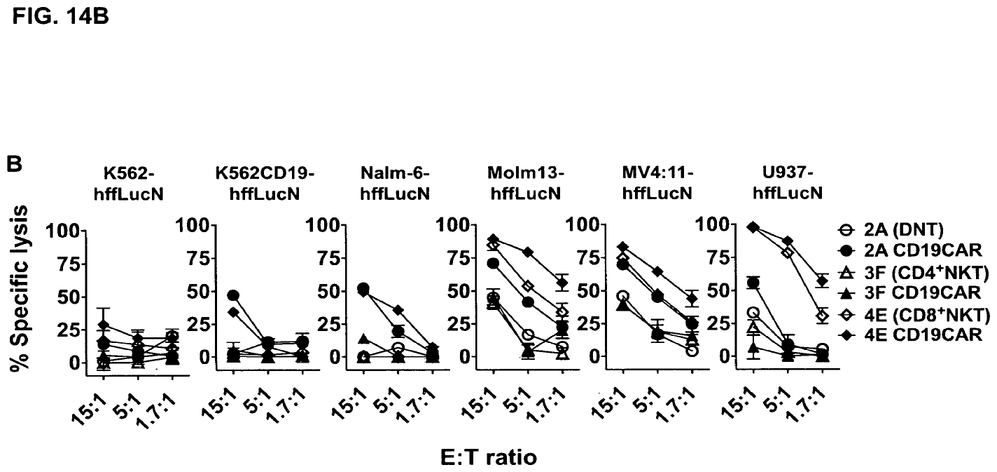

To evaluate whether CD3⁺CD56⁺ NKT cells modified with a CAR or TCR can redirect NKT cell specific targeting of cancer cells, 2A (DNT), 3F (CD4⁺ NKT), and 4E (CD8⁺ NKT) clones were spin-transduced with CD19 CAR lentivirus supernatants. FIG. 14A shows the CAR gene transfer efficiency in the 2A, 3F, and 4E clones. CAR gene transduction efficiency in the 2A (˜30%) and 3F (˜26%) clones was higher than in the 4E clone (˜4%) due to differential growth rate. FIG. 14B shows that both 2A and 4E clones transduced with CD19 CAR killed CD19⁺ Nalm-6 B-ALL and K562CD19 target cells. Expression of granzymes A, granzymes B and perforin as well as CD4 and CD8 molecules in CD3⁺CD56⁺ NKT cells support the notion that genetic modification of CAR or TCR in those cells can redirect their cytotoxic specificity against cell surface or intracellular antigens for cancer treatment.

The foregoing embodiments and examples are provided for illustration only and are not intended to limit the scope of the invention. One of ordinary skill in the art will recognize the numerous modifications and variations that may be performed without altering the spirit or scope of the disclosure. Such modifications and variations are encompassed within the scope of the disclosure.

1. A method of treating or preventing a cancer, comprising administering to a subject in need thereof a therapeutically effective amount of isolated CD3⁺CD56⁺ type III natural killer T cells, wherein the cancer is a solid tumor or a hematological malignancy; and wherein optionally the cancer is resistant or refractory to treatment in the absence of the cells.

2. The method of claim 1, wherein the cancer is selected from a B-cell malignancy, leukemia, lymphoma, myeloma, or melanoma; acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma; and wherein the cancer is resistant or refractory to treatment in the absence of the cells.

3. The method of claim 1, wherein the cells are CD3⁺CD4⁺CD56⁺, CD3⁺CD8⁺CD56⁺, or CD3⁺CD4⁻CD8⁻CD56⁺ cells, each optionally isolated from a biological sample of the subject or a donor.

4. The method of claim 3, wherein the biological sample comprises blood, bone marrow, lymph node tissue, spleen tissue, tumor tissue, one or more induced pluripotent stem cells, and/or one or more peripheral blood mononuclear cells; and wherein optionally the blood comprises peripheral blood and/or umbilical cord blood.

5. The method of claim 1, wherein the cells are isolated from one or more peripheral blood mononuclear cells.

6. The method of claim 1, wherein the cells are modified to express a chimeric antigen receptor (CAR) and comprise one or more polynucleotides encoding the CAR.

7. The method of claim 6, wherein the CAR comprises at least an antigen binding domain, a transmembrane domain, and an intracellular signaling domain.

8. The method of claim 7, wherein the antigen binding domain is capable of binding to CD19, IGF1R, ROR1, BCMA, CD123, CD33, CD38, CD138, CLL-1, LILRB4, GD2, CD20, CD22, CD30, MSLN, EGFRvIII, EGFR, HER2, MUC1, EPCAM, PSMA, SLAMF7, GPC3, or PD-L1.

9. The method of claim 7, wherein the antigen binding domain is capable of binding to CD19, and the cancer is a B-cell malignancy, e.g., B-cell precursor acute lymphoblastic leukemia (B-ALL), non-Hodgkin lymphoma (NHL), or chronic lymphocytic leukemia (CLL); or wherein the antigen binding domain is capable of binding to ROR1, and the cancer is Ewing sarcoma, osteosarcoma, fibrosarcoma, rhabdomyosarcoma, chronic lymphocytic leukemia, mantle cell carcinoma, breast cancer, lung adenocarcinoma, melanoma, neuroblastoma, or ovarian cancer; or wherein the antigen binding domain is capable of binding to BCMA, and the cancer is multiple myeloma; or wherein the antigen binding domain is capable of binding to CD123, CD33, CD38, CD138, CLL-1, or LILRB4, and the cancer is AML; or wherein the antigen binding domain is capable of binding to EGFRvIII or EGFR, and the cancer is glioblastoma; wherein the antigen binding domain is capable of binding to GPC3, and the cancer is hepatocellular carcinoma.

10. The method of claim 7, wherein the antigen binding domain comprises an antibody or an antigen binding fragment thereof, or a non-antibody protein scaffold; wherein the antibody is a monoclonal antibody, a polyclonal antibody, a synthetic antibody, a human antibody, a humanized antibody, or a single domain antibody; wherein the antigen binding fragment comprises a single chain variable fragment (scFv); wherein the intracellular signaling domain comprises a functional signaling domain of at least one stimulatory molecule.

11. The method of claim 10, wherein the at least one stimulatory molecule comprises a zeta chain associated with a T cell receptor complex or a CD3 zeta chain; wherein the intracellular signaling domain further comprises a functional signaling domain of at least one costimulatory molecule; and wherein optionally the at least one costimulatory molecule comprises 4-1BB, CD28, CD27, CD134 (OX40), ICOS, DAP10, or DAP12.

12. The method of claim 1, wherein the cells are modified to express a T cell receptor (TCR) and comprise one or more polynucleotides encoding the TCR; and wherein the TCR comprises at least an alpha chain and a beta chain.

13. The method of claim 12, wherein the alpha chain and/or the beta chain is capable of binding to an antigen, and wherein the antigen is an intracellular antigen, NY-ESO-1, WT1, or MAGE-A3.

14. The method of claim 13, wherein the antigen is NY-ESO-1 and the cancer is neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, or breast cancer; or wherein antigen is WT1 and the cancer is AML.

15. The method of claim 1, wherein the cells are modified to express a T cell receptor mimic antibody (TCRm) and comprise one or more polynucleotides encoding the TCRm.

16. The method of claim 15, wherein the TCRm comprises at least an antigen binding domain, a transmembrane domain, and an intracellular signaling domain; wherein optionally the antigen binding domain is capable of binding to a composite antigen comprising a peptide and a human leukocyte antigen (HLA) molecule; and wherein optionally the HLA molecule is a class I or class II HLA molecule.

17. The method of claim 16, wherein the peptide comprises an alpha fetoprotein (AFP) peptide.

18. The method of claim 17, wherein the composite antigen comprises an AFP peptide and a HLA-A2 molecule; and wherein the cancer is hepatocellular carcinoma.

19. The method of claim 16, wherein the peptide comprises a preferentially expressed antigen in melanoma (PRAME) peptide; wherein optionally the PRAME peptide comprises SEQ ID NO: 41; wherein optionally the composite antigen comprises a PRAME peptide and a HLA-A*0201 molecule; and wherein the cancer is B-ALL, AML, multiple myeloma, T cell lymphoma, melanoma, non-small cell lung cancer, colon adenocarcinoma, or breast adenocarcinoma.

20. The method of claim 16, wherein the peptide comprises a WT1 peptide and optionally a HLA-A2 molecule; and wherein the cancer is AML.

21. The method of claim 16, wherein the antigen binding domain comprises an antibody or an antigen binding fragment thereof, or a non-antibody protein scaffold; and optionally wherein the antibody is a monoclonal antibody, a polyclonal antibody, a synthetic antibody, a human antibody, a humanized antibody, or a single domain antibody; optionally wherein the antigen binding fragment comprises a single chain variable fragment (scFv).

22. The method of claim 16, wherein the intracellular signaling domain comprises a functional signaling domain of at least one stimulatory molecule; wherein optionally the at least one stimulatory molecule comprises a zeta chain associated with a T cell receptor complex or a CD3 zeta chain; and wherein optionally the intracellular signaling domain optionally further comprises a functional signaling domain of at least one costimulatory molecule.

23. The method of claim 22, wherein the at least one costimulatory molecule comprises 4-1BB, CD28, CD27, CD134 (OX40), ICOS, DAP10, or DAP12.

24. The method of claim 6, wherein the cells are further modified to comprise an exogenous cytokine, growth factor, antibody or antigen binding fragment, or any combination thereof, wherein the antibody or antigen binding fragment optionally comprises a bispecific T cell engager (BiTE).

25. (canceled)

26. (canceled)

27. (canceled)

28. A pharmaceutical composition comprising isolated CD3⁺CD56⁺ type III natural killer T cells, which are modified to express a CAR, TCR, or TCRm, and a pharmaceutically acceptable carrier.

29. The pharmaceutical composition of claim 28, wherein the cells are CD3⁺CD4⁺CD56⁺, CD3⁺CD8⁺CD56⁺, or CD3⁺CD4⁻CD8⁻CD56⁺ cells, each optionally isolated from a biological sample of the subject or a donor.

30. (canceled)

31. (canceled)

32. A method of preparing a therapy for treating or preventing a cancer in a subject in need thereof, comprising:
a. isolating one or more CD3⁺CD56⁺ type III natural killer T cells from a biological sample; and
b. culturing the one or more CD3⁺CD56⁺ type III natural killer T cells in a growth medium to produce an expanded cell population.

33. The method of claim 32, further comprising modifying the one or more CD3⁺CD56⁺ type III natural killer T cells to express a CAR, TCR, or TCRm, wherein the modifying comprises introducing one or more polynucleotides encoding the CAR, TCR, or TCRm into the one or more cells; wherein introducing one or more polynucleotides optionally comprises electroporation, transduction, and/or transfection; wherein optionally the one or more polynucleotides comprise mRNA and/or DNA; and wherein optionally the DNA comprises transposon DNA.

34. The method of claim 33, wherein the one or more polynucleotides comprise one or more vectors, wherein the one or more vectors comprise one or more viral vectors or lentiviral vectors or γ-retroviral vectors.

35. The method of claim 32, wherein the cancer is a solid tumor or a hematological malignancy; B-cell malignancy, leukemia, lymphoma, myeloma, or melanoma; acute myeloid leukemia (AML), B-cell precursor acute lymphoblastic leukemia (B-ALL), non-Hodgkin lymphoma (NHL), chronic lymphocytic leukemia (CLL), Ewing sarcoma, osteosarcoma, fibrosarcoma, rhabdomyosarcoma, mantle cell carcinoma, breast cancer or breast adenocarcinoma, lung adenocarcinoma, ovarian cancer, multiple myeloma, glioblastoma, hepatocellular cancer or hepatocellular carcinoma, neuroblastoma, metastatic melanoma, synovial sarcoma, bladder cancer, esophageal cancer, head and neck cancer, non-small cell lung cancer, prostate cancer, T cell lymphoma, or colon adenocarcinoma.
